(19) United States
(12) Patent Application Publication
(10) Pub. No.: US 2026/0191793 A1
(43) Pub. Date: Jul. 9, 2026

(54) HBV SPECIFIC EXOSOMES AND USES THEREOF

(71) Applicants: The United States Government as represented by the Department of Veterans Affairs, Washington, DC (US); EAST TENNESSEE STATE UNIVERSITY RESEARCH CORPORATION, Johnson City, TN (US)

(72) Inventor: Zhi Q. Yao, Johnson City, TN (US)

(21) Appl. No.: 19/127,762

(22) PCT Filed: Nov. 11, 2023
(86) PCT No.: PCT/US2023/079448

§ 371 Date: May 7, 2025

Related U.S. Application Data
(60) Provisional application No. 63/424,656, filed on Nov. 11, 2022.

(51) Int. Cl. A61K 9/50; A61K 31/506; A61K 31/52; A61K 31/522; A61K 31/7072; A61K 31/7105; A61K 38/46; A61K 47/64; A61K 47/69; A61P 31/20; C12N 5/071; C12N 9/22; C12N 15/11

(52) U.S. Cl. CPC A61K 9/5068; A61K 31/506; A61K 31/52; A61K 31/522; A61K 31/7072; A61K 31/7105; A61K 38/465; A61K 47/64; A61K 47/6901; A61P 31/20; C12N 5/067; C12N 9/226; C12N 15/11; C12N 2310/20

(57) ABSTRACT

Disclosed are exosomes comprising a Na+ taurocholate co-transporting polypeptide (NTCP) binding motif, such as HBV preS1 peptide (PSIP). Disclosed are methods of making exosomes that have been engineered to express a heterologous binding motif on the surface of the exosome, wherein the heterologous binding motif is a NTCP binding motif. Disclosed are methods of treating a subject infected with hepatitis B virus (HBV) comprising administering to the subject a therapeutically effective amount of an exosome, wherein the exosome comprises a NTCP binding motif and a therapeutic agent, thereby treating the HBV infection or any other liver diseases including liver cancer in the subject.

### CROSS-REFERENCE TO RELATED APPLICATIONS

This application claims the benefit of U.S. Provisional Patent Application No. 63/424,656, filed Nov. 11, 2022, which is incorporated by reference herein in its entirety.

### STATEMENT REGARDING FEDERALLY SPONSORED RESEARCH

This invention was made with government support under R21AI157909 awarded by National Institutes of Health. The government has certain rights in the invention.

### REFERENCE TO SEQUENCE LISTING

The Sequence Listing submitted Nov. 11, 2023 as an xml file named “37759_0594P1.xml,” created on Nov. 11, 2023, and having a size of 89,719 bytes is hereby incorporated by reference pursuant to 37 C.F.R. § 1.52 (e) (5).

### BACKGROUND

Hepatitis B virus (HBV) chronically infects nearly 300 million people worldwide, resulting in one million deaths annually, owing to liver cirrhosis and hepatocellular carcinoma (HCC). The current antiviral treatment can only suppress de novo HBV replication but cannot eliminate HBV infection due to the persistence of HBV covalently closed circular (ccc) DNA, which sustains HBV replication. Distinct from integrated HBV DNA that may encode only subgenomic transcripts (HBsAg), the cccDNA (an episomal viral minichromosome) serves as the genuine template for transcribing all HBV mRNAs, including the pre-genomic RNA (pgRNA) that serves as a reverse transcription template. Thus, HBV cccDNA is the key therapeutic target for eradicating HBV infection. Thus far, the molecular mechanisms of HBV cccDNA biogenesis in infected cells remain unclear, and there are no available drugs that can directly target HBV cccDNA. Therefore, novel curative strategies focused on eliminating HBV cccDNA are urgently needed to treat HBV-infected cells without inducing cytotoxic effects.

### BRIEF SUMMARY

Disclosed are exosomes comprising an HBV receptor Na+ taurocholate co-transporting polypeptide (NTCP) binding motif.

Disclosed are exosomes comprising HBV preS1 peptide (PS1P).

Disclosed are hepatocyte-derived exosomes comprising a Na+ taurocholate co-transporting polypeptide (NTCP) binding motif.

Disclosed are exosomes comprising NTCP binding motif fused to a transmembrane protein.

Disclosed are hepatocyte-derived exosomes comprising a NTCP binding motif fused to a transmembrane protein.

Disclosed are methods of making an engineered exosome comprising transfecting a plasmid into cells, wherein the plasmid comprises a nucleic acid sequence capable of encoding a fusion protein, wherein the fusion protein comprises a transmembrane protein and an extracellular target-specific binding motif; culturing the cells to allow production of exosomes expressing the transmembrane protein and an extracellular target-specific binding motif fusion protein; and obtaining an exosome-containing supernatant.

Disclosed are methods of treating a subject infected with hepatitis B virus (HBV) comprising administering to the subject a therapeutically effective amount of an exosome, wherein the exosome comprises a NTCP binding motif and a therapeutic agent, thereby treating the HBV infection in the subject.

Disclosed are methods of inactivating HBV DNA in HBV infected cells comprising administering to the HBV infected cells a therapeutically effective amount of exosomes, wherein the exosomes comprise a NTCP binding motif and a therapeutic agent, thereby inactivating HBV DNA in the HBV infected cells.

Disclosed are methods of inactivating HBV DNA in HBV infected cells comprising administering to the HBV infected subject a therapeutically effective amount of exosomes, wherein the exosomes comprise a NTCP binding motif and a therapeutic agent, thereby inactivating HBV DNA in the HBV infected cells.

Disclosed are methods of delivering a therapeutic agent to HBV target cells comprising administering to the HBV target cells a therapeutically effective amount of exosomes, wherein the exosomes comprise a NTCP binding motif and a therapeutic agent.

Disclosed are methods of reducing HBV production from cells infected with HBV comprising administering to the HBV infected cells a therapeutically effective amount of exosomes, wherein the exosomes comprise a NTCP binding motif and a therapeutic agent, thereby reducing HBV production in the cells infected with HBV.

Disclosed are methods of cleaving an HBV genome in a cell infected with HBV comprising administering to the HBV infected cells a therapeutically effective amount of exosomes, wherein the exosomes comprise a NTCP binding motif and a therapeutic agent, wherein the therapeutic agent is a HBV specific gRNA/Cas ribonucleoprotein complex comprising a HBV specific gRNA and a Cas protein.

Disclosed are methods of inhibiting HBV replication in an HBV infected cell comprising administering to the HBV infected cells a therapeutically effective amount of exosomes, wherein the exosomes comprise a NTCP binding motif and a therapeutic agent, thereby inhibiting HBV reactivation or replication in the cell.

Disclosed are methods of inhibiting HBV replication in an HBV infected cell comprising administering to the HBV infected cells a therapeutically effective amount of exosomes, wherein the exosomes comprise a NTCP binding motif and a therapeutic agent, thereby cleaving HBV genome and thus inactivating HBV replication and expression in the cell.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-C shows an example of designing gRNAs targeting HBV genes that are critical for HBV cccDNA and/or RNA biosynthesis. A) CRISPR/Cas9 gRNAs targeting HBV DNA critical for viral replication but do not overlap with the human genome. The gRNAs for Cas9 require two RNAs: trans-activating CRISPR (tracr-RNA) and CRISPR-RNA (crRNA). B) CRISPR/Cas12 gRNAs targeting HBV DNA. The gRNAs were numbered and named based on their predicted antiviral potentials by the e-CRISPR program. C) CRISPR/Cas13 gRNAs targeting HBV RNA, designed by the Sanjana Lab online tool, which takes into consideration the target RNA structures, isoforms, and mismatch rates, to blast and select specific regions of HBV RNA that Cas13 is guided to target and edit.

FIG. 2A-F show summary data for the suppression of HBV replication in HepDE19 cells by synthetic gRNA/Cas9 RNPs. FIG. 2A-2B show HBsAg and HBeAg levels in the supernatants of HepDE19 cells transfected with gRNA5 or gRNA9 RNPs versus control gRNAc, determined by ELISA. FIG. 2C-2E show pgRNA, HBV DNA, and cccDNA levels in HepDE19 cells transfected with gRNA5 or gRNA9 RNPs versus control gRNAc, determined by real-time PCR. Data are means±SE from three independent experiments carried out in triplicates. Values were normalized to the gRNAc-treated cells. FIG. 2F shows HBV mRNA levels in HepDE19 cells transfected with gRNA5 or gRNA9 RNPs versus gRNAc, determined by RNAscope. Representative RNAscope images (left) and summary data (right) are shown. The positive HBV RNA dots from 6 imaging areas were quantified by QUPath software and normalized to the gRNAc-treated cells. * P<0.05, ** P<0.01, *** P<0.001, **** P<0.0001.

FIG. 3A-B show summary data for the antiviral effects of synthetic gRNA/Cas9 RNPs. HepG2-NTCP cells were infected for 6 days with the supernatants of HepDE19 cells that were transfected with gRNAc/Cas9, gRNA5/Cas9, or gRNA9/Cas9 for 3 days, followed by measuring HBV DNA and cccDNA in HepG2-NTCP cells using real-time PCR.

FIG. 4A-B show summary data demonstrating that synthetic gRNA/Cas9 RNPs inhibit HBV cccDNA in HepG2-NTCP cells. HepG2-NTCP cells were infected for 6 days with the supernatants of HepDE19 cells. Next, HepG2-NTCP cells were transfected for 3 days with gRNA5, gRNA9, gRNA5+gRNA9, or gRNAc RNPs (FIG. 4A) or incubated with exosomes carrying gRNA5, gRNA9, gRNA5+gRNA9, or gRNAc RNPs (1×10⁸ particles packaged with 40 μM gRNA/Cas9 RNPs) (FIG. 4B), followed by measuring HBV cccDNA.

FIG. 5 shows representative data of a T7E1 assay to detect mismatch in HBV DNA. HepDE19 cells were transfected with gRNA5 or gRNAc RNP (upper panel), gRNA9 or gRNAc RNP (middle panel), or gRNA5+gRNA9 or gRNAc RNP (lower panel) and treated with (+) or without (−) T7E1. HBV cleaved products are shown (lower bands). Duplicates are shown for each treatment.

FIG. 6A-E shows representative data exhibiting features of the engineered exosomes. FIG. 6A is a schematic model of the engineered exosome packaged with gRNA/Cas9 HBV gene-editing RNP drugs and have surface PSIP to target HBV receptor (NTCP) on hepatocytes. FIG. 6B is a size distribution showing the size and concentration of the purified exosomes, measured by the Zeta View NTA. FIG. 6C is an electron microscope image showing the shape and size of the purified exosomes, observed by an F20 electron microscope (EM). FIG. 6D is an image of the immunoblotting of exosome markers (CD63 and CD81) using proteins (10 μg/lane) extracted from supernatants of cell culture before and after the exosome purification. FIG. 6E are fluorescence images after exosomes were incubated with HepG2 or HepG2-NTCP cells for 1 h, 4 h, and 16 h, followed by visualizing the amount of RFP-CD9-PSIP fusion protein using EVOS Auto fluorescent microscope.

FIG. 7 shows summary data for reducing HBV cccDNA biosynthesis by single or combinational gRNA/Cas9 treatment. HepDE19 cells were transfected with gRNA5, gRNA9, gRNA5+gRNA9, or gRNAc RNPs for 3 days, rested for 6 days, and then repeatedly transfected again 3 more times with the same gRNA/Cas9, followed by measuring HBV cccDNA using qPCR.

FIG. 8 shows representative data demonstrating that HBV infection persists in Fah/NRG-hu HEP mice. Mice were inoculated with HBV (10⁷ GE/mouse) and blood samples were collected at 2, 4, 6, 7, 13, and 16-week post-infection (wpi). HBV DNA levels were measured by qPCR.

FIG. 9 is a schematic showing HBV infection and drug administration in FRG-KO, liver-humanized mice for drug biodistribution and safety assays.

FIG. 10 is a schematic showing HBV infection and NP-TAF and/or EXO-HBV Eliminator treatment in liver-humanized mice.

FIG. 11A-E show representative data regarding the design and selection of gRNAs to target the HBV genome and inhibit HBV replication. FIG. 11A is a schematic showing that the E-CRISPR gRNA designer was used to compare the sequences of 8 HBV genotypes (genotype A-H) and identify 9 potential HBV gRNA target sites in the HBV genome (denoted by scissors), including polymerase (P), capsid (C), surface(S), and non-structural X genes. FIG. 11B is a plot showing the HBsAg levels in the supernatants of HepG2/2.2.15 cells transfected with synthetic gRNA/Cas9 RNPs for 3 days, determined by ELISA. FIG. 11C is a plot showing the HBeAg levels in the supernatants of HepG2/2.2.15 cells 3 days after nucleofection, determined by ELISA. FIG. 11D is a plot showing the HBV mRNA levels in HepG2/2.2.15 cells 3 days after nucleofection, determined by real-time RT-PCR. FIG. 11E is a plot showing the HBV DNA levels in HepG2/2.2.15 cells 3 days after nucleofection, determined by real-time PCR. Data shown are means±SE from three independent experiments carried out in triplicates. Values were normalized to the gRNAc-treated cells as a control. * P<0.05, ** P<0.01, *** P<0.001, *P<0.0001.

FIG. 12A-F show summary data for the induction of HBV replication in HepDE19 cells by tetracycline-free culture medium. FIG. 12A-B show the time-dependent induction of HBsAg and HBeAg levels in HepDE19 cells cultured in tetracycline-free (Tet-free) medium, determined by ELISA. FIG. 12C-E show the HBV DNA, cccDNA, and pgRNA levels in HepDE19 cells cultured in Tet-free medium, determined by real-time PCR. Data are presented as mean±SE from three independent experiments carried out in triplicates. Values were normalized to the gRNAc-treated cells as a control. * P<0.05, ** P<0.01, *** P<0.001, **** P<0.0001. FIG. 12F shows representative images and summary data of the RNAscope results showing HBV RNA positive signal (small dark dots) in HepDE19 cells cultured in Tet-free medium for 0 and 2 days. RNAscope imaging of HepG2-NTCP cells without HBV infection is shown as a negative control. The data were analyzed by QuPath software to quantify the positive HBV RNA dot area versus cell area from 6 imaging areas.

FIG. 13A-F show representative data for the suppression of HBV replication in HepDE19 cells by synthetic gRNA/Cas9 RNPs. FIG. 13A-B show HBsAg and HBeAg levels in the supernatants of HepDE19 cells transfected with gRNA5 or gRNA9 RNP versus control gRNAc, determined by ELISA. FIG. 13C-E show HBV DNA, cccDNA, and pgRNA levels in HepDE19 cells transfected with gRNA5 or gRNA9 RNP versus gRNAc, determined by real-time PCR. Data are means±SE from three independent experiments carried out in triplicates. Values were normalized to the gRNAc-treated cells as a control. * P<0.05, ** P<0.01, *** P<0.001, **** P<0.0001. FIG. 13F shows HBV RNA levels in HepDE19 cells transfected with gRNA5 or gRNA9 RNP versus gRNAc, determined by RNAscope. Representative RNAscope images and summary data are shown. The data were analyzed by QuPath software to quantify the positive HBV RNA dot area versus cell area from 6 imaging areas and normalized by gRNAc.

FIG. 14A-E show representative data for the Antiviral effects of synthetic gRNA/Cas9 RNPs. FIG. 14A-B show the detection of HBV DNA and cccDNA in HepG2-NTCP cells 8 days after HBV infection using the supernatants of HepDE19 cells transfected with gRNA5, gRNA9, or gRNAc for 3 days, determined by real-time RT-PCR. FIG. 14C shows the data for HBV-infected HepG2-NTCP cells transfected with gRNA5, gRNA9, or gRNAc for 3 days, followed by measuring HBV cccDNA levels by real-time RT-PCR. Values were normalized by the gRNAc-transfected. ** P<0.01, *** P<0.001. FIG. 14D shows the HBV cccDNA levels in HepDE19 cells transfected with gRNA5, gRNA9, or gRNAc for 3 days, and the same treatment was repeated 4-times. DNA was isolated and HBV cccDNA levels were determined by real-time RT-PCR. * P<0.05, *** P<0.001, **** P<0.0001. FIG. 14E is a T7E1 assay to detect mismatch nucleotides in HBV DNA from HepDE19 cells transfected with gRNA5 or gRNAc (upper panel), gRNA9 or gRNAc (middle panel), and gRNA5 plus gRNA9 (lower panel) and treated with or without T7E1. The cleaved DNA products are shown (lower bands).

FIG. 15A-B show representative data for the DNA sequencing of gRNA5+gRNA9/Cas9 RNP-treated HepDE19 cells. The cell treatment, genomic DNA isolation, and target gene amplification are described in the Methods section. Sanger DNA sequencing of the PCR products was performed using primers flanking region covered by the gRNA5 forward primer (FIG. 15A) and gRNA9 reverse primer (FIG. 15B). The sequencing data were aligned and compared with the scramble control (gRNAc), which is identical to the HBV DNA sequence database (ayw strain, Genbank accession number: NC_003977.2). The Chromas DNA sequencing software was used to read the nucleotide peaks, and the alignment of two or more sequences was performed using BLAST tool (from NCBI-BLAST) to compare the nucleotide sequences. The representative HBV DNA sequencing data with gRNA5 and gRNA9 targeted sites as well as the nucleotide substitution mutations are shown.

FIG. 16A-D show representative data for the cytotoxic effects of synthetic gRNA/Cas9 RNPs. FIG. 16A is a cell viability assessment by MTT assay of HepDE19 cells transfected with gRNA5, gRNA9, or gRNAc and cultured in the presence or absence of Tet-free medium (for HBV induction) for 24 h. FIG. 16B is a cell viability assay of HepDE19 cells transfected with gRNA5, gRNA9, or gRNAc and cultured in Tet-free medium in the presence or absence of 10 ng/ml of TNFα. FIG. 16C shows Flow cytometry analysis of apoptosis in HepDE19 cells transfected with gRNA5, or gRNA9, or gRNAc and cultured in the presence or absence of Tet-free medium for 3 days, determined by Av and 7ADD staining. FIG. 16D shows the proliferative ability of HepDE19 cells transfected with gRNAc, gRNA5 or gRNA9 and cultured in the presence or absence of Tet-free medium for 3 days, determined by MTT assay. Data are means±SE from at least three independent experiments. Values were normalized by the gRNAc-transfected cells. * P<0.05, ** P<0.01, *** P<0.001, **** P<0.0001.

FIG. 17A-E show representative data of NSG mice treated with exosomes carrying synthetic gRNA/Cas9 RNPs or PBS-Cas9 control. Mice (6 per group) were injected (via tail vein) with either 100 μl (0.9 nmol gRNA/Cas9 RNPs) exosome drugs or PBS every 3 days for 6 weeks. Peripheral blood (PB), spleen, and liver were collected at the end of the experiments. The plasmas were used for detection of Cas9 protein and antibody as well as inflammatory cytokines. Spleen and liver were used for H.E. and TUNEL staining. DNase I (1,000 U/ml, Invitrogen) treatment was used as positive control for TUNEL staining of apoptotic cells.

FIG. 18 shows a procedure for engineered exosomes. Alternatively, GFP-Cas9-expressing construct can be co-transfected together with pCT-CD4BL-CD9-GFP into 293T cells and collect CD9-exosomes with both GFP and RFP expression and transfect these exosomes with sgRNA only. RFP-positive exosomes can be transfected with sgRNA and followed by transfection in vitro-synthesized Cas9 with protocol published in Pharmaceuticals 2021, 14 (4), 356;://doi.org/10.3390/ph14040356

FIG. 19 shows the structure of CD9 and the cloning strategy.

FIG. 20 shows the NTCP sequence.

FIG. 21 shows identification of preS1 binding protein on primary hepatocytes expressing NTCP. Predicted tsNTCP protein sequence. A 30-amino acid insertion unique to tsNTCP is underlined. Two peptides identified by LC-MS/MS were highlighted in green. All lysine and arginine are highlighted in red to indicate trypsin cleavage sites. Many of the potential tryptic peptides are not appropriate for LC-MS detection because of unfavorable size and/or hydrophobicity. PTH: primary Tupaia hepatocytes; MS: mass spectrometry.

FIG. 22 shows the sequence of HBV pre-S1 gene.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an exosome” includes a plurality of such exosomes, reference to “the therapeutic agent” is a reference to one or more therapeutic agents and equivalents thereof known to those skilled in the art, and so forth.

The term “exosome” refers to cell-derived vesicles having a diameter of between about 20-160 nm, such as between 30 and 150 nm, preferably a diameter of about 80-120 nm.

As used herein, the term “mammal” is meant to encompass, without limitation, humans, domestic animals such as dogs, cats, horses, cattle, swine, sheep, goats and the like, as well as non-domesticated animals such as, but not limited to, mice, rats and rabbits.

The word “or” as used herein means any one member of a particular list and also includes any combination of members of that list.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” HBV may refer to inhibiting survival, growth, and/or spread of the virus. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Exosomes

Exosomes are bi-lipid membrane vesicles of about 20-160 nm that are secreted by many cell types. Exosomes can be isolated from any suitable biological sample from a mammal, including but not limited to, whole blood, serum, plasma, urine, saliva, breast milk, cerebrospinal fluid, amniotic fluid, ascitic fluid, bone marrow and cultured mammalian cells, induced and non-induced pluripotent stem cells, fibroblasts, platelets, immune cells, reticulocytes, tumor cells, mesenchymal stem cells, satellite cells, hematopoietic stem cells, pancreatic stem cells, white and beige pre-adipocytes and the like. Exosomes can include specific surface markers not present in other vesicles, including surface markers such as tetraspanins, e.g. CD9, CD37, CD44, CD53, CD63, CD81, CD82 and CD151; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LAMP2 (lysosome-associated membrane protein) and LIMP (lysosomal integral membrane protein).

Disclosed are exosomes that have been engineered to express a heterologous binding motif on the surface of the exosome. In some aspects, a heterologous binding motif can refer to a binding motif that did not originate from the exosome. For example, a heterologous binding motif can be added to an exosome using recombinant engineering. In some aspects, a binding motif is a sequence that is capable of binding to a target sequence. For example, a Na+taurocholate co-transporting polypeptide (NTCP) binding motif is capable of binding to NTCP.

Disclosed are exosomes comprising a NTCP binding motif. Disclosed are hepatocyte-derived exosomes comprising a NTCP binding motif. Disclosed are hepatocyte-derived exosomes comprising an HBV preS1 peptide (PSIP). In some aspects, the PSIP is the pre-S1 domain of the large HBV envelope protein that can be essential for binding of HBV to its receptor expressed on target cell. Thus, in some aspects, only the region of PSIP responsible for binding to NTCP is present on the exosomes. In some aspects, the PSIP on exosomes is used to target exosomes to an NTCP expressed on human hepatocytes. In some aspects, amino acids 2-47 of full length PSIP can be used as the NTCP binding motif. For example, amino acids 2-47 consist of GTNLSVPNPLGFFPDHQLDPAFGANSNNPDWDFNPNKDHWPEANQV (SEQ ID NO: 1).

In some aspects, the PSIP comprises a sequence 50, 55, 60, 65, 70, 75, 80, 85, 90, 95 or 99% identical to SEQ ID NO:1. In some aspects, the NTCP binding motif can be a fragment of PSIP, wherein the fragment of PSIP retains NTCP binding function.

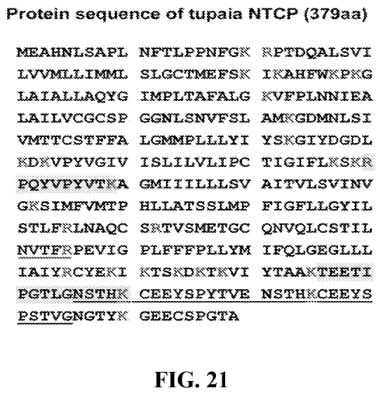

Disclosed are exosomes comprising a PSIP binding motif. Disclosed are hepatocyte-derived exosomes comprising a PSIP binding motif. In some aspects, the PSIP binding motif can be a NTCP. Thus, also disclosed are hepatocyte-derived exosomes comprising a NTCP (e.g. a PSIP binding motif). In some aspects, the NTCP can be essential for binding of the exosome a cell expressing PSIP. Thus, in some aspects, only the region of NTCP responsible for binding to PSIP is present on the exosomes. In some aspects, the NTCP on exosomes is used to target exosomes to a PSIP expressed on human hepatocytes (that have been infected with HBV). In some aspects, the NTCP sequence as shown in FIG. 21 can be used as the PSIP binding motif. In some aspects, the NTCP comprises a sequence 50, 55, 60, 65, 70, 75, 80, 85, 90, 95 or 99% identical to the sequence of FIG. 21. In some aspects, NTCP comprises all or a fragment of the protein encoded by GATTAAAGAAGGCATCCAGCAAGAACTGCACAAGAAACGGAGTCAGCCGGAGAAC AAGGAGTGGTCTTCCACTGCCTCACAGGAGGATGGAGGCCCACAACGCGTCTGCCC CATTCAACTTCACCCTGCCACCCAACTTT GGCAAGCGCCCCACAGACCTGGCACTGAGCGTCATCCTGGTGTTCATGTTGTTCTTC ATCATGCTCTCGCTGGGCTGCACCATGGAGTTCAGCAAGATCAAGGCTCACTTATGG AAGCCTAAAGGGCTGGCCATCGCCCTGGTGGCACAGTATGGCATCATGCCCCTCAC GGCCTTTGTGCTGGGCAAGGTCTTCCGGCTGAAGAACATTGAGGCACTGGCCATCTT GGTCTGTGGCTGCTCACCTGGAGGGAACCTGTCCAATGTCTTCAGTCTGGCCA TGAAGGGGGACATGAACCTCAGGTAGGCCTGGGGGTGAGGAGGGAACAGCCTGGG ACAGGAGCTACATTAGATATGTCACAATTGAGGGGAAGAGCTGGGCTAAAGCCAG GAAAAGGAGTGAATGCAGGCTGGGTGGAGGCTAGGGCAGGGGAAGTCTGGCATTT ATTGCCAAGAGCTTATGCTGGGATGGGGGCAGGAGTTGGGGTACG TCTGGTTTAGGCACCAAGCCAAAGGGACCATACTGGTCCAGGAAGCTCGTGGTGAT CTCCTTGCTTCCAGAGGTGTCTCAAGGATTGTCTTAAAGGAGCACTGATTAAAGCCA AAGGAACCTCTGGAGAACTGCATCCACCTACAACAGTGGACAAGTTGCCTGCTTTG AGCCCAGAGATTTAAAAGGTAGTGTGATTCGTTCCAAACAT CATCAGGGAGATCAAGGTGACTTGGAACCAAATGAGCACCTCAAATTCCTGCAAAG CGGGCAAGAATTTCCAAAAGGTCCTCCCAATTACCTGGTCATCTAAGATGTTTTCTC TTTAAAGTCCCTCATTCCCCATCAGATGCAATGACATTTCCATGCAAAGGCACAACC AGGGAGATCCTAGAGTTTCCCAGCACCCACTCCAGATAGGCCAGCCCCATCTCAGC CACCCTGATAAGGGGAAGACAGCAGCACCCCCTAACCCCCTGCCCAAAGCATTA TAAGCAGAAATCAGCAAGGGCTCGCTCCTGGAGACGCAGCACACAGGGGCTGGTTG ATGCTCATCCTTCCAAACTGGGGAGACTCTAGTCTTGAACATCTGCCAGAGGAGCTA CTTCAGACCTGCTCCAGCTCCAGCAGC CTGTGCTGAAGCTAAAGAAAACTCACTAGCAGAAGCTCTTGCTTTCAGTGAAACCT CAGTCTCAAGCCAG TCCCCCCTGGTTTAGTCCTCCGTGGAGACGCTATTCAATGCCAGGTATAGCAGCAGA ACCCAGAAAGTCCATCTGCTTGTGTCTGTCAGCACCCTACTCCTAGCCAAACTTTCC TAGGAAAACTAACCCAGTCAAGAAGCCCCTCCTAGTGATGCAAAATGACACTGGGA CAGATTTTTTAAATGAAAAAAGTGAGAATGGCTGGGCACG GTGGCTCACACCTATAATCCCAGCACTTTGGGAGGCCAAGGCAGGAAGATCACTTG AGACCAGGAGTTTA AGACCAACCTGGGCAACATAGCAAAACCCTGTGTCTACAAATATATACATATATAT TTAATTAGCCAGGCATAGTGGCACATGCCTGTAGTCCCAGCTACTTGGGAGGCTGA GATGAGAGGATCTTTGAGCCTACGAGTTCAAGGCTACAGTGAGTCATGATCACACC ACTGCACTCCAGCCTGGGCAACAGAGTGAGACCCTGTCTCTC TCTCAAAAAAAAAAAAAAAAAGGAAATAAAAAAGTGAGAGTGAATGAGAGTTGGG GTTTAACAGGATCTGCCATCTGAGAAGCTCTGGGCTCTTTTTCCATATGATTCAGCC CTCACTTTAGTAGTCAATGGGAAGAGAGGCACAAAGAGGGCATTTGGCTTGTGCCA TCACCCACGGAAAGGTATTTCAATGTTCAACTACCTCACTGT GAGAAAATGCTTTCCTCTGTCTAATCCACATCCTCTCTGCTACAAAGCAAGCCCTTG GTCTTGGATCTGGCTTCTGGACAAGGCTCATGGCAGCCTGTGTGTCCCAAGAATCCT AGAGTATATCTGAATTTTGGTGTATGAGGGATGCAGAAGTCAGGGGCCTGCGAAGG GGTACTCTGAGGGCTTTGTGTTTCTGTGGAAGAAGCAGAG CAGGGGACGTGGAAGGCAACTGTGCTTTTAAGTGTGAGTGGCCAGATGTCCCAGCC CCATCGCCAGTGGCCACTATTGAGGCCTCTGCCACAATGCCTACATTGCTGTGGCTC TAGGCCCCACCCAGGCAGAGCCTCTGGTGACTCAAGAACATGACCCCTCATGCTCT CCTGCTCAACCTCCTTGTCCAGACCCCTGATGACTGTCATT TTTACCTAGACCCCCACAAGAGATAAGCTCTGCTGGGACAGCCACCTTCTGCCATAA AACTCCTTCCCAACAAACAACCCTCCCATGCACATTAAACCATGTCTCAGATCCCAC TGTCGGAATGGGAATCTGGTCATCTACCTTCATGTGCTGCCAGACTTCAAATCCCAT TCCAATTTGGGACAACTTGTTACATGCATTATAATAATGACAACGATTATAAGAAAC AGCCAGGCACAGTGCTAAACACCTTACCTGGATAAGTATATGTGATCCTCCT ACAGCACCCTGGGGGACAGCATGTAACAAGTAGCATTTAGAGGTAAGGAAACTGA ATCTTAGAGAGGTCAATACATTGGCTCCAGGTCTGGGTGGCTCCGGAACCTGTGCTT TTACTGAAATATCTTCCAGGTCAGCATTTCCACAAACAAAATGGCCAGACAGCCTC AAAGGCATGGGCAGACACCCGTGTCTCAGCAGCAAGTATTGT GTGGGAGCCAGAGCTTCCCACTTTTCAAAAGAAGGTAAAATATCCATTTTATGTGA AATCAGCAACTCATCCATTTTTTTAAATGTGCAAACCAAACAATACATGTCAAAGG GCCAGCTGTAGCCCAAAGGCCCCAGTTTGAGATAAGCACAATACTCTACATCCCAT TTAGAACTGGAAGGAATTGTAAGACACAGATCCCATTTTAC ACATGAAGAGGCTGAGGTCAGAGACTTGCCAGAGGTTTCCCAGAGTTTGTGGGAAA CTGTGTCTCCCTCCACTCACTCTGCCTCATAGGTGGAGTCAGTCTGGGGACAGCCTG CCATCCCAGGAGGCCAGGCCACCAAGCAGACCCCTCAATGCAGCCAGGGACGGCA GCTCCCTCCTCAAGGAAGGCCACTAGACAAGTTTCAAAAAATGTAGGTGACAAACA GGCAAGAGAAAACGCCCTCACAGTTGAAGTGAGTATGGCTTGTCAGGCACAGAGA AGAGAGTGCTATGCCAGCAGGAACTGGCCTCTTTTGACCATCCTTGCTGCCCCAAGT TCATCTGCTCCCCCTGCCACCATCGGACTCACAGCAGAGCCCAGAGCCAATGGCCA TGGGGACAATGGGCTTGGACACAACCCAAGCACTTCCACAGGTCTCCCAGCCATGA TGTAGGAAGCTCAACCAGGACAAGATGGAAGCCACTGGGGTC CCAGAAGTTTTCCACTTCATAGAGACTGTGTTGAGGCCCCTCACAGCTCCCTCAGCT TCTGCCTCCAATCTGTGCATTGACGGACACTCGAACGAGTCCCTTAAATGGGGTACA CAGATGTCCAAAAGCAAGGTCTATGCTGTGCTCTTGGAACTTAAGCAGGAGGGAAA AATAAAAAAAACAAATGTGCCCCTTTCCCCCTGAAAGGCA ACCAGGCCTACAATCTCAGGAATCTAGAGTCTGAAAAAGTCCTTGCCCGTCCCTGTC CCTTGGGGTAAACCAAGGATCACAACAGTCACGGATGCCAGCAGGCTAGGAGATG GATGTCCCTGACCTGACCCCAGGACCTGTGCAAGTGGAGGAAGTACCTGGGGATCA GGGAACATGGCTCCTGCATACCCAAGCTGACAAGGCACACCA AGTCCAAACTCCAAAACTCCTGACACACAAGCCAAAGTAAGCATGATTCCTCTGCT CTAGGTCACCAGAACAAACACACAAGTTGTGTTTGGCTTTTCCCTAAGAGAAGATC AACAGTAGCATTGAAACCCCCTGTATTCCTATTCCCAATCTCATTACCCTCCACCAC TTGCCAAAGGGAGCATCCCTGGGAATTTGGTGTTCAGCATT TCCACCGACCCTTTTCAACTATTATATACGTATATATTCATAAACAAGATAGACTGC GGGATTTACGTCTTTTAATTTTGTACGAACAGTATTATACTGTCCACATCACCTAAC AGCTTGCTTTTTTTTTTTTTTTTTGAGAAATAGGCATGTAAAGAAAATAGCTACCTCA TGTATCTGAACTGCTGTGCAGTTTGCTTGCATTTATCCATTCTCCTGTTGTCAGATTC ATGTGGATTTTACGTCTCGCTTCTGTGAACAGACTTGTGTGTGTCCCTCTGCCCACAT GAGGGCATATGCAATTGAAGCAGGACTGCTGGGTCGTGGGAGTCACACATCTTTAA GAGGTCTCCAAGGTCATTGTTCACTTCACACGTTCCCTAGCAGTGTCTGAAGATCCC TGTTTCTCTGTGCCTTCATCTATATTTGGAATTGTCAGACTTTTTAATTTTTTCCAAAT GAAAACAGTGAAGCAGATCGCAGTAGTTTTAAATTTGATTTCATGGGAGGCCTACC ACATTTCCACGTGTGCGTTGGCCATTCATGTGTCCTCTTCTGGAAACATTGGGTCCTT TGCCTGTTTTTCTATGGGATTGTGGTTTGGGTGGGTTTTGAAAATTAATTTCTAAA GTTATTGTTTTATATATGTTGCAACAGTCTATTCACAGTTTGGAGCTGGTCTTTACAC TTTGATAATAATGTCTGATAGATTGCTTATGTTAATTTAGTCAAATTTGCCAATATTT TTCCTTTGTGGTTTATGTTTTTTAGGGGTTTCTTTAAGAAATCTTTTACTATCTACAA GCCTGTCCAACATGGCAAAACCCCGTCTCTACTAAA AAATACAAAAAAATTAGCTGGGCATGGTGATGTGCACCTGTAATCCCAGCTACTCA GGAGGCTGAGGCATGAGAATCACTTGAACCCAGGAGGCAGAGGTTGCAGTGAGCC AAAATCATGCCACTGCACTCCAGCCTGGGCGACAGAGCAACACCCTGTCTCAAAAA AAAAAAAAAAATTTACTGTCTACAAAGTTACAAAGATATTACC CTGTATTTTCTTTGAAAAGTTTTAAATGTTTGATTTTCACATGGAGAGCTCCAATCAA TGTAGAACTTATTTTTATGTATGGCATGAACTAATTTTATCTTTTCCATACAAATAGA AGATTCCAAGCCACGTTGAGTGAATTTTTCTCCAGGTATTTGTAATGACATCTCTCT CATAAATGCAGGGCTTGTGTCTGGATCTCTCTTCTCTTCTACGGGTCTATGTGGCTAC CCTTGTACCAGTTCCACACTTTCTTAATTACAATTTTATAAGTTTTGTT ATCTGAAAGTTAAGTCCCCCTACTTTGTTCTTGAAAATCATGGGCCGGGCACAGTGG CTCACGCCTGTAA TCCCAGCACTTTGGGAGGTCAAGGCTGGCCGATCACGAGGTCAGGAGATCGAAACC ATCCTGGCTAACAC GGTGAAACCCCGTCTCTCCTAAAAATACAAAAAATTAGCTGGGCGTGGTGGCGGGC GCCTGTAGTCCCAGCTACTTGAGAGGCTGAGGCAGGAGAATGGCGTGAACCCGGGA GGCAGAGCTTGCAGTGAGCCAAGATCACACCACTGCACTCCAACCTGGGTGACAGA GCGAGACTCTGTCTCAAAAAAAAAAAAAATCATGTTAGCTAT TCTTAGCCTTTTACTCTTTCACTTGAGTTTCAGTATCACCTTTCAAGTCTACAAAAAA AATTATAAGTCTGGAAGTTTTATTAAATGTGCATTACATTTATATATAAATTGAGAG ACTATCTTTATAATATTGAATCTTCCCATCTATAAATATGATATAGCTTTTCTTTTAC TTAGGCCTTTTTTAATACCTTTCTATAAAGGTTTGTA ATTTCTTTCATAAAGATCTTACATGTCCTTTAGATGTATAAGTATTTTCTAGTTTTGT GAATATCATAAATGGTGTCTTTTAAAATATATATATATTTGTTTCTAGTATATCAGA CCCAATTGATTGACTGACTGATGCTAATGTATTTAGAACCTCATACATTTTCTTACTT ACCAGTTATAATGGAAAATTACTTAGATTTCTTCTGT GGACAATCCTATCATTGGCAAAACAAGAAAGTTTCATTTCTTCTTTTCCCATCCCTA TACTTTTTCTATTTCTTGTTTTGCTGTGCTTACTAGGGCCTATATATAGTATAATTTAT AAAACAGGTAATAATAATGACTATCTTTGCTTTATTCCTCTTGTTAAAGAAAATCCC TCTATTTACCATTAAGTTTAACGTTTAATATACTTTT TCTAATAGATTTCCTTTCAAGTTAAGGAATTTTCATTCCGTTGCTAGTGTACTAAATT TTTATTATGTAAGAACATTAAATTTTGTTGACTTTTCTATAGCTACAAAATTGTACCA TTTCTTTAATAATATAGTAATATGTATTAATAGATTATCTAATGTTAAATCATTCTTA CTCATCTTGTTCATTGTATTTTTTAAAACACTTATTTGCTAATATTTTATTATTTTTGT TTCTATGTTCATAAATAAGAGTGGTCCCCAATTTTCTTTTCTCGTAA TATCATTCTCCAGCTTTCGTTCCAAGATCATATTAACTTTATACAATAAATTAGGGC ATGTTTCCCCTTTTTATATTATCTGGACAGATTTGCATAATATTGCAACAATCTGCAC TTTGCATGTTTAGTAGAACACTACCGTAAGACAGTATAAATCATGTAGGGTTCTAGA AGATAGGTTTTGAACTGCTTATTAAATGTCTTAAATGA TTCTAAGTTGAATCAGGTTTTCGGCTTCATATTGACTTGGTTTTGTAGGTTATATTTC CCATGAAATTGTCCATTTAAGTTAAAAATGAAAGTATGAAAATGTCCTTAATGACTT TTTAAAATCTTCATTTTCTCTGTAGTTATGTTCCCTTTTTCACACTAACCTCATCAATT TGTGCCATCTCTCTTTTCCCTGGTCTGCCTTGCCAG AGGTGCCTATTTAATTGGCCTTTAAAAAAAAAAAGAAAAAAAAAAAACCCTATTTT TGGTTTTACTGATTCTAACAATTTACAAAACTTAAAAAATTTCTTCCTTTTCTTTTTA TTGGGTTTTTCTCATTCTAACCTCTTAAGGTGCTTAACTCATTAATTTTTAATCTTCTT TTTCAATATAAATATGTTAGGCTATAAATTGCCCTTTAATAATTGCTTTATTTGCTCC CTGCATGGTTTTTAATTACTATTTATTGAGATATAATTCACATACCATA AAATCCATTCTTTAAATTGTACGATGCCTTGGTTTTTAGCATAATTCCAAGCCTGTG AACCTATCACCATTATCTAATTTCAGAACATTTTCAATACCCCAAAAAGGAACTCCA TCCCATTTAGCAGGAGTCCCCATTTCTCTCTCCTGCTCCAACCCTTGACAACCACTA ATCTTTCTGTCTCTATGGATTTCACATAAATGGAAGCAT TCAATATGTGACCTTTTGTATCTATACTTCTTTCACTTAGCATATTTTCAAGGTTCAT TCATGTGATAGCATGTCACAATACTTCATTTGTTTTTGTAATTGAATAATATCCCATT GTATGGATATAATACATTTTGCTCATCCATTCATTCAGCTGATGGACACATGGGTTG TTTCTACTTGTTCCCTAGTATGAATAATGTCTATGGA AGCTCACAGGCAAGTTTTTGTGTGAACATGCTTTCTGTTCCCTTGGGATATATACCT AAGAGTAGAATTGCTGGGTTATATGGTGACTCTGTGTTTAAATTTTCTAGAAACTGC CAAACTATTTTCCAAAGCACCATTTTACTTCCCACCACCAGTGTATGAGGATTCCTA ATTTTCCATATACCCTCCAACACTTGTTATTGTCCAGCT TTTTAATTATAGCCATGCTAGTAGATATAAAGTGGTATCTCAAAGTGGTTTTGATTT GCATTTTCCTAATAAGTAATGATGTTGAGCATCTTTTCATGTGCTCATTAGCCATTTG TATATCTTCTTTGGGAAAATATCTCATTCAAATCCTTGGCCCATTTTTTACTTGTTTTT TATTATTGCATTATAAGAGTTCTTCATATATTGGCC GGGCGCGGTGGCTCACGCCTGTAATCCCAGCACTTTGGGAGGCCCAGGCGGGTGGA TCACGAGGTCAGGAGATCGAGACTATCCTGGCTAACACGGTGAAACCCCATCTCTA CTAAAAATACAAAAAAAATTAGCCGGGC GTGGTGGCAGGCGCCTGTAGTCCCAGCTACTCGGGAGGCTGAGACAGAAGAATAGC GTGAACCCAGGAGGCGGAGCTTGCAGTGAGCCGAGACTGCGCCACTGCACTCCAGC CTGGGCGACAGAGCAAGACTCCATCTCAAAAAAAAAAAAAAAGAGTTCTTCATATA TATTCTGAATATTAGACCCTTATCAGATATATGACTTGCAAA TATTTCCTCCCATTCTCTTGTTGGCTTTTCACTTTCTTGATGGTGTCCTTTGAATCACA AGTTTTTAATATTAATGAAGTCTGACTTGCCTTTTTCTTTGGCTGTTCGTGCTTTAGA TGTCTAAGAAACCATTGCCTAATCCAAAGTCCCCTCTCCATGTTTTTTAAATATGTAT TTTATTATTTTTATATTCTAAATACTGTTTCCACCATGATTTCATATTTGATGATATA ATTAGAAAGTTCCCCTCCCCTCATCCCATGATCCTTTTATTGGTTGC TCAGTTTCAATGCTCCTCCCATCCCAGGTCTAATACCTCAATTCTGGTCCCTTCCTTG GGCATTAAAGCCTCCACAGTGCCAAGACTAACACCCCACTCCCCTCCAGAACCCCT CTATGAAGCTTCAGCTTGAGTTCCCCCACTACTTTGCATTTGTTTCTTGTCCGTGGGC ACCTTTTTTTTTAGTGGAGTGGCGCTAACATAGCTCAC TGTAACCTCAACCTTCTGGGCTCAAGCCATCCTCCTGCCTCAGCCTCCCAAGTAGCT GGGACTCCTGGTGCACACCACCACACCTGGCTAATTTTTGTGTGTGGTTTTTGGTTT GTTTGTTCATTTGTTTGTGTGTAGAGACAGGGGTCTTACTTTGTTGCCCAGGCTGGTC TCAAACTCCTGGCTTCAAGCAATCCTCCCTCCTCGGCCTCCCTAAATGCTGGAATTA TAGGCATGAGCCACTATGCCACACTTAAATTGAGTGTTAATTTCTTACAAGCTCAAG TGTGTATTTAAGTGTTCACGGGGGTGGGGGAGTTCCTATTACCTAGTTCACAATTTT CTTTGAGGGACTCTTCTCACCTCTAACAGCTCTTATCCTTATGACATGCCTACCTATT GTTCTTGATGGCTACGCTCTGGAACCTTTGATATAGGCTGCTCTTCATGCATATTTAA GCCATATTCCATCTTTATAGAAATAATGAAAGAAGTGTAGATGGAGGGTAGGGCTG TGGGGAGGAGGAGGGACACACTGAGTGTGGTTTTCCTAAGATCTTGCCAAGCCCTC ACCTCCCAACCATCTTCCCACTGGAATAAAAGTCAGAGCCATATATTGTTAATGTAA ATGTCCATAAATCTAAGGAGATGCCCTATCAGCTTCCTTCTCAGTTGACCTTTCTGG AGGGCCTCTCCCTAGACAGGGTCCTGGGACTAACATATATAAGATTCCAAATGGAA AAGGAAATTGGACAAGTAGGATACATATTAGAGCTGGAAGGACATATTATTCAGGC TCTGAGTGTCATAATTTATTTTAAATAATATCAAGAGGAACCAGAAAGTAAAGAAC ATGAGAACACCAGAGTTGGGGACTGGAATACTATCCAAGATGCAAAGCAAATCTGA CCAGGTTATCTTGTCACTTACTCAACATGGGTATCTATTACTGCCTTCTCTACTACCC CACCATTATGGCCCTTCAATTATTCTGTTTCTTACTGCTTCACTATTTCTTTTACAGTC CTTTTAATCATGGTATCTGTTTATTGATTTTTCTAGTAGGGTTTTTGACTTCTGCCTG AGCATCACTCCCATGCCACCCATCCCCAAAGACCAAGTGTCCTGTCACTGTGTCTCA CACTCAGACTCCCCACGAGGTAAAATGTGCTGATATAGCTAATTGTTATTGTCTCCG TTGGACAAAGTCACTACAAGAAGCCATTCCCTAGGCTCTAAACCAGACATTTAGTA ACTGTGGGTCAGGTGCCATTCCATGGTCCAATGGGCTAATGACCAGAGTAATGGAA TCACACAGTTGGCTTTCTACAAAATGGGCTTGGCATGGGGGAGGTGCTCAATGCGA AAAGGGTTCCATTACAGAGAAGGAGATCAGAATGGCTCCTTCCACAATGAAGCACA CAGGGATTGAAGCAGTTGAACCACCCTAAGTTCAAATGCTGCCTTGGCCACTTACTA CCTTGTGCGACTTTGAGCAGGACACAACTCTTCTAGTCCCCAGCTTCCTTGTCAGCG AAAAGAAGATAGTATGCTTACTGCCTAATGTTATGGGGAAAAGGAACTGAAGCCAC ATATGTCAAGCACTTAGCACAGTGGCACTTAGCAGGCACTCAACAAAATAGTCTGT ATTTATGGGCTCTCTCTCTCTCCCCTCTTCCCATGTCTCCCACCAGCATTGTGATGAC CACCTGCTCCACCTTCTGTGCCCTTGGCATGATGCCTCTCCTCCTGTACATCTACTCC AGGGGGATCTATGATGGGGACCTGAAGGACAAGGTGCCCTATAAAGGCATCGTGAT ATCACTGGTCCTGGTTCTCATTCCTTGCACCATAGGGATCGTCCTCAAATCCAAACG GCCACAATACATGCGCTATGTCATCAAGGTAAGAACCTGGGAGGCTTGACAAATTA GGGCAAAAGAGTAAAAACACCATATACAACTATAAGATATAATCATTATATTAATA ATAAACATACATATGCTCCTACTAGATGCTAAGTTTTATGCTATGCACTCTAAGGAT TCACTGTCACCAGAGTTTGATATTTGCAGAAAGTGAAGTAACACATGAGGCTGG CGTGGTGGCTCACGCTTATAATCCCAACACTTTGGGAAGTTGAGGCGGGTGGATCA CTTGAGGTCAGAAGTTTGAGACCAGCCTGACCAACATGACGAAACCCCGTCTCCAT TAAAAATACAAAAAAAAAAAAAAAATTAGCCGGGCATGGTAGCGAGTGCCTGCAA TCCCAGCTACTCAGGAGGCTGAGGCAGCAGAATCGCTTGAACCAGGGAGGGCAGG GGTTGCAGTGAGCCGAGATCGCGCCACTGCACTCCCGCCTGGGCCACAGAGCAAGA CTCCATCTCTAAAACGAAAAAAAAAAAAAAAAGAAAAGAAAGAAAATGAAGTAAC ACACGAGAGAAACAAGATCCAATTCCAGTGTACACGCAAGTATTTGCAGAAGGCAA GGAGATGGGAACAGTGAGGGCTGGAGTGGTCAAAGAGCCTTATTAAAAAGGAAGG GCTTGAGGCATTTAGGTAAACCTGGGGAGAAGCCTGAATAGAGTTCAGCAGGCACT AAGGGCAAATTTGGCTGATGTGGCAAGAAGCTAGAGGAGACAGGTAGTGTACCCAT TCCCAGCATTTCAAGAGAGGGATGGGGTGGGAAGTGGAGTGAATTAAGGCCACAA AGGTTTTGAGAAGCACTGTGGTCACAGACAGGGCATCATGCCACTGCTTTGTGTGC AGAACTCCTAGAGTGCAGCTATGGTCTCTCCACCAATATTTTCAACCTTGGAGTTTG GTCCCTGCCAGCCTCCCCCTTGGCTCACTATTCATCCCCCAGGGCCAGGAGGTCATC CTGGTCCTCAGGAAGGACTTGACCTCCTGACCTCCTGTACCATCTCTGCCCCTGGAT CAATGCCTCTGGCCTGGTCTGCAAGGCGTGCTGCTGAGAACCCCAAAGTTTGGCAG CAGCTTTGGGCCTGGAACAAAGAAGTCAAGAGTCCAGTTTTCAGGGGGGTTTTATTT TCACACTACCATTGTCTCAAGAGTAGTTTTTGCCCTTGTCCTGTCCCAGTGCCATGGC CACAGCTCCCCTTCCCGCCTGCTGGAGGACATGGAGGAATC ATCCCTCCTCCTTTCTGTGGCCAGTCACCCCACCTCAGCCCTGGATTGCAATCATTCT CTCTACCCTTGTACTCAAGAGACCTCCTTTTATTGATTATGCCCCTGGCCCAGCAGC CTCCCCTCTCTCTCATTTCTAAAATATCTCTGTATTGGTTCTTCACAATCAGCTTTGA TACGAACTCACATATCTTCTATCTTCAAAAAACAAAACAAAAAGTGTCTCTCACTGG GATTTTTTGTGGAATCCACCTCCCATTCCACCTAATATCTCCCTTTGCTCTCCTTCCT AGCCAGGTTTTAGGAGTAGTCCCCCTCACTCCTCTCTCTCTCTGTCAATCGCTTCCTC TCAAACACCTTCCATTTCCCCCATGCCACCGCCAGAGCTCTCTCATGAGTGACCAGG CCCTCAGCAATTCGTTTCCATTATTTGAATCCCTCTCTTCTCTCCTTGGTCTCCCATCC ACCCAGCATCTTGATGACCACCTGCTCCACTTTCTTTGCCAAATCCAATGGACATTT CTCAATCCTTCCTTGACCTTCTAGCAGCATTGGACATAATTGACCACTGCCTCCTCA AAAGCCTTCTCTGTCAGCTTTAGTGACTCTAAGCTCTGCTGCTTTCCTCCTACCTCTC ATGACCTTCAGACTCCCTTATGAGTTCCTCTGGTCAAAGGCACTTCTCTGCATTCAG ACCAAGCTTTCTGCCTTTCTCACCAGACCTCTCCCAGCACAGGATCTGGTCCCTCTC AGGTCTGCAATTACCATCTGCAAACCGAGACTTCCAGAACAGCATCATCAGATGTC AACTCTATCCTCACTCCAGAATCCATTTATTATCTA CCAGGTCTTTCTATCTGAATATACCTCAGACATTTAAAACTCAATATATGTTAAAGT CATCTCCTCCCCATGTGGTTATCTCAGTCGGTGGCACTGTGTGTGTGCCCAGTTGCC CCAACCAGAAACTGGAAGTCATTCTCAATGTTCCCTTCTCCCTCACCACTCACACCC ATCTAATCAATGACTGTCGATTCCACCTTCTGTTGCTGGAATCCATTCATTCCTCTCT ATCCTTACCTCTCACTAGACAACTCAAATGGCCTTCTCATCCAACCTCCTC ACCCAGTTCTAACCTGAGGGTGAAGATGTGAAGTACAAACTCTATCATGTCACTCC ACTGCTTAAAGCCTCTCATTGACTCCTTCCCCCTAGGATAGTTTAAACTCCTTAATGT GGCACTTCTTGATTTGGTTTCAACTGACCTCACCAGTCTCAGCTCTTTTCTCTTTGCA CGTGCCCTTCTCTTCACCTGGAACACTCTTCTTCCCAATTCTCACCCCACTCACCATC TTTCACTGGATCACTCCCACTCATCCTTTAAGTCTCAACACAAATAGCGC CTCCCCCAGGAAGCCTTCCTTAACCTCCTAAGCCTGAGTAAGATGCTTGTCCTATTC ACTCCCATAACACCCAACACTCCAGCTAACCCCTGTAGCAATTATTACACTGCATTA AGAACATCTGTTCATTTTTATTCCCCACTCATTTATCAAGAAGCAAGACCATAAGGC TGTCTTAATTAACACTGAATCCACAGCACCTATCACAGTGCCCCCATAGCTAATGCC TGAGAATAAATAAAGGAATGAAGGGCAAGTCCTGTCTGCTGCTCTTCCACAC ACCAGCAGAAAAACAACATACACAGAGAAACATGTTTGTGTGCATCATTTCATTTTT TTAAGATAGTTTGTGGGGTGCATAAGATTGCTTTTTTAAAAAATCCTATACACCCAT AAACGATCTTAAAGAAATGCATATAAAATATAAAGAATATTAAAATGTGAAGTGAC CACTGCTATCATTTAATGCTACTCTGCCCTCCTCCATTTTATAGGTGGGAAAACTGG GAAACAGAATACCAAGTCATAGTGCTAATGACAACTGATGTGAGAACATGTCT CTTTTCTACCTCAGAGTAGCTTTAAAAGTGTTTCTCTGAGGTTGCAAAAAGCAGAAT CTCAGACCCTTTTTTTTTTTTTTTTTCGAGACGGAGTCTCACTCTGTCGCCCAGGTTG GAGTGCAGTGGCGCGAGCTCGGCTCACTGCAAGCTCCACCTCCCGAGTTCACACCA TTCTCCTGCCTCAGCCTCCTGAGTAGCTGGGACTACAGGCGCCCGCCACCATACCCG GATAATTTTTTGTATTTTTTAGTAAAGACGGGGTTTCACCGTGTTAGCCAGG ATGGTCCCGATCTCCCGACCTTGTGATCTCCCCGCCTTGGCCTCCCGAAGTGCTGGG ATTACAGGCGTGAGCCACCACACCTTGCCGAGAATCTCAGATCTTAAAGCTGGAGA AGCCTTTAAGATGAAGTTCAGTCTCCTGCCTTATTCCCAATTTGCAGATAGAGTAAC CGAGGCCCGGAAAAGTTACTTGGCAGAGCCAGGATTATTTCAACCAGTCTGTTCCT GTTGTGTTGCACATTCACTACTCAATGCCACAGACGAAATGAAGTCAAAATGAA GCCAGAGAACATTAAAAGGGCTAAGAAATACCTTGGCTGGAAAAACAGAGACTAG TAATAAGGGACTACATAAAAGGTAATCTAAAAAACCCAGGCATGCCCTGGACCCAT TTTGCCCCTGGCTAAACCTCCAGGGTTAAACCACCACAAAGTCAACAGCTACACTG ATCTTCCTATATTCCAAAACTGGAATCACTCCTGTGAGGATGCAGAGACTAACATGT GGGTCAAGCTGCAAAAGCTAAGGTTTCCTTGCTTCTATGGCTCTTCGAGGACTATGC TCATCCTTCGTCACCCTGTCCCATAAATAATATAGTGAGTGCCAAACTGGAAAACGA ACAAATGATTGTCCTAGCCTCTTACGCACACATAACCCTCCTTGGACCTTGCAAATC AAAATAACCCAACTTTCAATAACTAGCTCTGGCAGCAGGTATCTGCTCACACCGGG TTCAACCAGTTGCATTCGCTGGTGGAAGGTCACATTTAGATATACCAAGATACAATT TCAGGCATGCTCATTTTAAAAGGCAATTCAAGTAATTTTTAACTTACCCATGCAAGG GTTTATCCAAAGGCTATGTGGCTAAAAGGGTGATGAATCTGAGAATGACATGGTCC AGGAGCCACTTTCACCATAAGCCATCCACCACTCTTTAATAAGGATCCCACCATTTC ATTGACTCAACACTCTACTCATGCATTTCCCACACCTGCTGCTACTGCTGAACTGAC CCCTAACTCTCCTTTGTCTTACAACTTCTGCCAACTTGTGGCTATTGCTCCCAGACCT CTGTTCTGACACTTGTCCCTGCTATTGCTTCCCTCTCTGTAATCCTGCAAACTTCCTA AATGAGAGAATCTGATTATTTCTACCACTCACCAGCCTGTGCGGGCCACCTTTGTGG GTAGACTGTGGACATGTCATCTCATGTGTAAGCCAATGGATTGGCTGCCCATGAATG AGCACTCATCCCAGTIGTGAGCAGGGCAATGAAGTCAGGTGGTACAAAATGTGGTA GCCTATGGAGAAGCAACTACCCGGGGCCACTTGTCTCAGCAGGAGACAGGGGTTGT AGACAGGATAAATTTCCAGAGATGGCAGCAGTGAGAGTGACAGGAACCTCTAGAA TGTTAATTCCTCCCCTTTGAGTAATAACAGGAGTTACAGTTTAGTGAGCGGTTCTTA TATGCCAGGTCCTATGCCGTCAACCTCACTCAGGTTGAGTGACTCCCCAGAGCTTAC ACACCTGGAGACTAGCAGAGGCAGCTTTCCCACCAGGATCATATCAAATTATGTGC TCATAACCACAACTGTGCAGTCCCCAAGAGTCAGAGAACATGAGTGAGAGAATAAG AGTGAATGCCTATACAGAAGAAGAGGGCTTCTGGATTCGTGGACAATTCCTGTAAC TCAATGTCTCCTACATTATCCACTCCACTCTTGATAGAGATTAAAAATTATTTGAC TGTTGGGTAAAGGACAAATTCGAAAGACAGAAATCTAAAAGGGGAAATCAACTAA AAATGCAAAGGTAAACTGAGAAGTTCATTAGCTAATAAGTCTTTTAATAAAACTTG AAGCACATGGTTATTCAAGAGATGTGTGAAAGTCCTTCCTATGCTGGGATAGGACC ACTAATTGGTATAGGTGATGTTTTATATGATCATATCAGTTGCAACAAGTATTAAAA ACAGAAACACAAAGCTGTGCAGCTGAAATTTGAGCAATATAAATGTTAAAATGTCG TTAGGACATTATGAATGTACTTAATGCCACTGAACTGTACACTCAAAAGTGATTAAA ATGGCAAACTTTGCTATACATATTTTACTTTTTTAAAATGTTACTAGGAAATATAAA ATGCAACACATGTGAATGAACCAAAAAATGAACCAACACATGTAACTATTACAAAA TAACATGGTAAGTTTGAGAAAAGTTAAGTATTTCTTTACTCAGGAGATAAAAGGGA CAGTCTTAAATTTACACATACCTGTTTATCATTTTGGAGGACGCAATTCTCTAAAAT GCACACTGGAAAGTACAAAGTGCATGGAGTGCACATGCACTTTGAAAGGTTGCATC CTATGTTGCACAATGAAAAACCCTATAATTTCATTCAGAGTAGACCTGCATAGTTTC CAATGATGTTGTTATCACCCAAGATATTTCTGAAAGTTCTTTTGGGAACTTCCTTCAT ATTCAATTTGCAGGTCACTCAAGAAAGTAGGTTTGTCCCCTTATTATTAGACATCAT TTTGGGTTTAAAACCGTATTTCCGGGCTTGATCATCCACCTTGTTTACCAGCTTTGTT TCAAATGACTCTTGGCAATTTCCAGAATTGATCCTAAAATGGATAAAATCCCTATCT GTAATCAAGGTATTTGAGACAAATATGTATGACAAGTTCTCATGAGTTTCAAAGTAT GTGGAACAATGACAGTATTGTAGGAATGTGTATATAAGTTCCAAAAATACTTGACT TTTAAATAATATCCATTTTGCAGGAGTCCTGAATAGCCTCACGTAACACTTCACTGA AAGTCTGCTATCCAGAGATGTGTCAAAAGCAGTAACTGTGCTTGGCTGAGTTTGTAA TAATCAAAGTGTTAATCAGCTGTCCCCAAGCTTCCTAGCTACTGATACGAAATCTAA AAAGATGGGCTGGGTGCAGTGGCTCACACCTGTAATCCCAGCACTTTGGAAGGCCA AGGCAGGAGGACCGCTAAGAGTCCAGGAGTAAGACCAGCCTGGGCAACATAGTAA GACCCTGTCTCTAATGTTAATAATTTTAAAAAAATTTAAAGAAAGGAATCCAAAAA GAAGATAAAGGTCTTTCATATTCCTTTCCACTTCCAGTGTTTTATTGCAATGTTTCCC ACCTCCAATTTCTACCTGTGCTTCCCCACCTCTGTTCCTCTCTATCCCCAACTCTTCT AATTGCCTTTTCTTGTCTTCATTCCCAGGGAGGGATGATCATCATTCTCTTGTGCAGT GTGGCCGTCACAGTTCTCTCTGCCATCAATGTGGGGAAGAGCATCATGTTTGCCATG ACACCACTCTTGATTGCCACCTCCTCCCTGATGCCTTTTATTGGCTTTCTGCTGGGTT ATGTTCTCTCTGCTCTCTTCTGCCTCAATGGACGGTAGGTATTTTTTTTTTTTTTAAGA AAGGGTCTCACTCTGTTGCCCAGGCTGGAGGGGAGTAGCACATTCTCAGCTCACTG CAGCCTCAACCTCCAGGGTTTAACTGATCCTCCTGCCTCAGCCCCCCAAGTAACTGG GACTATAGGCACAAGCCACCATGACCAGCTGATTTAACTTTTCTGGAGACAGGGTTT TACCATGTTGCCCAGGCTAGTTTTGAACTAATGAGCTCAAGTGATCCACCTGCCTCA GCCTTCCAAAGTGCTAGGATTACAGGTGTGAGCCACTGCATGCCTGGAGATAGGTA TTATTTTAGGTCCCACTGTCCCATGGGCTTCATTCTTGTCCTAGTCTTTCAGGAAACG AATAAGAAGCTAGGAACCTGAAATCTGGGCAAGACACTTTCCTGGCAATATGTTCA GATGCACTAACTGGACAAGAGACTTTAGACTTGAAAAAAATTGAATGAAGGTATTG AACGGGTTAAAAATCCAGACACCAAAGGTATCCAAACACCAGCAGCAATAGCAAA GTACCCCTGTCCAGGGTCTAGATTCCATCTGCTGCGAAACTCTTTGGTAGCAGCACT GGGACAAAGTTGCTCCCTCCAGTTCCCTCTGAGTGTATGTGGGGTTTTCTTCACCGG CACAGGTGCAGACGCACTGTCAGCATGGAGACTGGATGCCAAAATGTCCAACTCTG TTCCACCATCCTCAATGTGGCCTTTCCACCTGAAGTCATTGGACCACTTTTCTTCTTT CCCCTCCTCTACATGATTTTCCAGCTTGGAGAAGGGCTTCTCCTCATTGCCATATTTT GGTGCTATGAGAAATTCAAGACTCCCAAGGGTGAGTACTGAAATTATCCCCACTTC AAGTTCTGCTCCAATATTAGACCTGAGCCTGCCAACAAGCAAGTTTCTAGCAGGGA CTTTTTGGATAGGGCTTTAACTCTTATCAAGTCACTAAGAACCAGGTAGCCCCAAGA AGCACCCAAGAAGCATTAAAGATCCAAGACTACTTCCATCAACTGCACTCCATTTCT TCCTTCTAGCTGGGAGAATGAAATTGCAGATGCAGTTTACCTCATAAAACTGAAGC AATTTGGGCTGCTTTGTCTCAGTGGGGAGACATCATAGGACAAAACTGCCTTGGAC AACTGAGGTTATTAGTTGTGTGAAAGGCCTCTATTCGCAAGAGTTGGGGAAGTTTCC ATTTCTCAGGACTAGGTTCTCATTTCCTGGAACCAGATCAGAGCCACAGCACCACTG GCACCTCCGTATGAGGAAAAACATATGAAAGCAATGCACCCTAATATAACCCAGAA TAAAGAAAAAAAAAAAAGATTCCTCAACTCTAGTTACGCAGTAAAGGAAACTTAAG CATACGGTTGTTAAACAGTAACTACCTCCACAATAAACTTAATATGATTTATACTTT ACTCTGATCACTATGGGGTTAACATAGCATCTCTTCTCTAAAATGCTCAGAGTATTA CCTTTCAAAATAAAATTGAACCTCAAGACAGACCAGAGAACAGAGTGCCTTCCTCA AAGTGCACTTTAACACCAATTTTAAAAAAAAAAAAAAAAAAAAAAAAAAACAGGA CATTCAAACCCACTTTATAACACATTTCATTATAACTCTCAACATTAGTATATTCTTC CTAAGTACCTACCCAAATCCTTCAACTGTCCCTTCATGTCCATGAATTGAAGTCCCC TTCAAAACCTTTCTAGACCCTAGGGACTGTGATTTACACCCTAGGAGAAATACACA ACTTTCCCTATTGGAATTTGTTACATTATACTAGCAAGCAAATGCCGATTCTCTCTCC CTTTGCAATTAGCTAAGATGTAATTAGGTTGCCAAGCAGCCAACACGAAACAAGTA CAGGCTGACCATACACTAGAACCATTTTAGAAGCTTTGGTTCAAAGCTACAGTAGA GTTGTGAGAAAGAATCTACTCTGATGCTTATCTGAGAGGAACGCAAGAGATTTGCT GCCCTCTTACAAAAAGTGCACAGAAGTTTCTAGACCAACCAAATTCCCAGATTTGA GGTTTTAGTACCAGAAAGACCTGAAGTGACCTCAAAGGACAATACTCTATGGATCT TTAGCTCTCAAGACTTGAAAAGAACTTTCCTCACAGTGTATCTCAGAAGACCCCAGT GGAAACACTAAATCAAAGCAACGTATTTCTTTGGACCTAATGGCCCCAAGAAGCTA TTCTCAGGGGGGAGGAATCTGTCAAGTTTTTATACTTTTCATTCCTGCACCTGATGG GAGAACAACTCTGTATAGAGAACTCTCCCATCTGTATAGAAAGGTGCAGGAATCAA AGCTATCTTCAAATTTGCATCACCGCCTACACCACGGACAAGAAGAGGTAGATCAA TTGGGGGTTGGAGGGAAGAACTGGAAACTCTCTTGGTTAGACTTTCTCGAAGATCTT TCCTGCCTGCCTCCTCATTCCCTGAATGCAACACAGACCTTGCATACATAAACTCAT CTCCTGTTGTTTTACTGTATAACGAAGTTAGAAGTGAAGTGATGATGAAGACACAAT AGTCTAATTCATTAGGACTTTTACTACTAGTACTACCATTAGGGAGACTGAGGGGAA AGTGAGTTTAAAGAGTAAACCATGGGAGAGACGACAGAGATGGAAAATATATACT TAGCACACTCTAGGGCTGAGTTGGTATTTTTGTACTTTCCAGATTAAACAAGCTTCA TTGTTCACTCCTCTTGTTCTCTCTCTACACCTGTAACCCTTTTTAACTTTACAGATAA AACAAAAATGATCTACACAGCTGCCACAACTGAAGAAACAATTCCAGGAGCTCTGG GAAATGGCACCTACAAAGGGGAGGACTGCTCCCCTTGCACAGCCTAGCCCTTCCCC TGGTGGCCTGGATTCTGGTCCCAAAGCAATTCTGAAAGCCAGTGTGGTAAACTAGA GAGAGCAGCAAAAACACCAGTCTTGCCTGAGTCTTTCTCCAGCATTTCCAGTACATC TATCAGAATCATCAAGTCTTGGCCGGGAACACAGACAGGGTGTCTACCCAAGAAGC CTCACCTATCCCCAACTTAGAATTTGCTACTTATTTTAAAGACTTGTTCAGTGACTGT AAACTCTATGAAACCAGAAACCGAATCTGCCTCTTGCTGGGATCTCTAAAAGTGTCT GATAAGCATCTTAAAGTCACTCAATTCAACTAATCAATATATATGTTTAACCCATTA CTCAAATACCCAAATCCCATTCCAAGTTTTGTGACCCAAAAGAGAAATAAATGCTC ACAAGTGCTGTAGAATTAAACTTCAGAAGTTCTAACCTAAAAAGTTCAGATCCTATT CCTTCCCTTTTGACATTATTGGGATGATGCTCCCGAAAAGTCAAATTTGACATCAAG TATGCAAAAGTGAACACAGTAAGATGCAATCAGGCAAAACAAACTCAAAAAATAG CTAATGAAATGAAAAAACTGGGCGAATGCATCATGTTAGTAGAGGAGGAAAACTTT TGACAAGGAAAAACCAGGAAACAAACACATACATTAACACAATGTTACCTCACTAA TAATCTCTTTTTTAAGTTCAGTAGGTATTTGGTTTTAACACAAGGCGCCCCCAAAAC TGGGTACAGCAAACTACTGCCAAAATGAGTCGTTTCCCAATTCAAAGAAAAATGTT CAAATACCTAGAATTCACAAATTTCAAATTGTCTCTAATAAAATTTAAACATTTTGC ATATCA (SEQ ID NO:13). In some aspects, the PSIP binding motif can be a fragment of NTCP, wherein the fragment of NTCP retains PSIP binding function. In some aspects, a fragment of NTCP can be any fragment of the sequence of FIG. 21 retains PSIP binding function.

Disclosed are exosomes comprising a NTCP binding motif (e.g. PSIP) or PSIP binding motif (e.g. NTCP) fused to a transmembrane protein. Disclosed are hepatocyte-derived exosomes comprising a NTCP binding motif (e.g. PSIP) or PSIP binding motif (e.g. NTCP) fused to a transmembrane protein. In some aspects, the presence of a transmembrane protein allows for the NTCP binding motif or PSIP binding motif to be positioned, or anchored, on the outside of the exosome. In some aspects, the transmembrane protein is CD9, CD63 or CD81. Disclosed are exosomes comprising a NTCP binding motif or PSIP binding motif fused to a transmembrane protein wherein the transmembrane protein is CD9. Disclosed are hepatocyte-derived exosomes comprising a NTCP binding motif or PSIP binding motif fused to a transmembrane protein wherein the transmembrane protein is CD9. Disclosed are hepatocyte-derived exosomes comprising a NTCP binding motif or PSIP binding motif fused to a transmembrane protein wherein the transmembrane protein is a truncated CD9. For example, a truncated CD9 sequence can comprise a CD9 amino acid sequence wherein amino acids 1-37 at N-terminal of CD9 are deleted. Such a deletion can me made such that that PSIP can be present on the surface of the exosome. For example, primers designed to a vector from System Biosciences (product Cat log No: CYTO123-PA-1 Plasmid name is pCT-CD9-RFP) can be used to truncate full length CD9 and provide a truncated CD9 with amino acids 1-37 removed. In some aspects, full length CD9 can be human CD9 having the sequence of

(SEQ ID NO: 14)
MPVKGGTKCIKYLLFGFNFIFWLAGIAVLAIGLWLRFDSQTKSIFEQETN
NNNSSFYTGVYILIGAGALMMLVGFLGCCGAVQESQCMLGLFFGFLLVIF
AIEIAAAIWGYSHKDEVIKEVQEFYKDTYNKLKTKDEPQRETLKAIHYAL
NCCGLAGGVEQFISDICPKKDVLETFTVKSCPDAIKEVFDNKFHIIGAVG
IGIAVVMIFGMIFSMILCCAIRRNREMV.

Thus, in some aspects, a truncated CD9 removing amino acids 1-37 can comprise the sequence

(SEQ ID NO: 15)
DSQTKSIFEQETNNNNSSFYTGVYILIGAGALMMLVGFLGCCGAVQESQC
MLGLFFGFLLVIFAIEIAAAIWGYSHKDEVIKEVQEFYKDTYNKLKTKDE
PQRETLKAIHYALNCCGLAGGVEQFISDICPKKDVLETFTVKSCPDAIKE
VFDNKFHIIGAVGIGIAVVMIFGMIFSMILCCAIRRNREMV.

In some aspects, the NTCP binding motif or PSIP binding motif is expressed on the surface of the exosome. In some aspects, the surface expression of the NTCP binding motif or PS1P binding motif allows the exosome to target and bind to cells expressing NTCP or PSIP, such as hepatocytes cells. In some aspects, the presence of a transmembrane domain fused to the NTCP binding motif or PSIP binding motif allows the NTCP or PSIP to be expressed on the surface of the exosome.

In some aspects, the NTCP binding motif is a full length or fragment of HBV preS1 peptide (PS1P). PSIP is well known in the art to bind to, or interact with, NTCP, therefore, in some aspects, PSIP is a NTCP binding motif. In some aspects, the PSIP is the pre-S1 domain of the large HBV envelope protein that can be essential for binding of HBV to its target. Thus, in some aspects, only the region of PSIP responsible for binding to NTCP is present on the exosomes. In some aspects, the PSIP on exosomes is used to target exosomes to an NTCP (e.g. on a hepatocyte. In some aspects, amino acids 2-47 of full length PSIP can be used as the NTCP binding motif. For example, amino acids 2-47 consist of GTNLSVPNPLGFFPDHQLDPAFGANSNNPDWDFNPNKDHWPEANQV (SEQ ID NO:1).

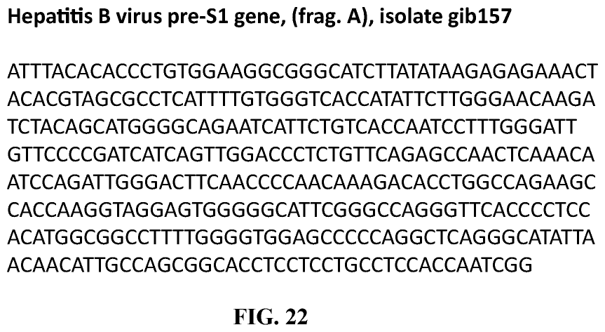

In some aspects, full length PSIP can be used as the NTCP binding motif. For example, full length PSIP comprises the amino acid sequence encoded by the sequence of FIG. 22. In some aspects, the fragment of PSIP can comprise 50, 55, 60, 65, 70, 75, 80, 85, 90, 95, or 99% sequence identity to the amino acid sequence encoded by the sequence of FIG. 22. In some aspects, the fragment of PSIP can comprise any fragment that retains NTCP binding function. In some aspects, the PSIP comprises a sequence 50, 55, 60, 65, 70, 75, 80, 85, 90, 95 or 99% identical to the amino acid sequence encoded by the sequence of FIG. 22. In some aspects, the NTCP binding motif can be a fragment of PSIP, wherein the fragment of PSIP retains NTCP binding function.

In some aspects, the PSIP binding motif is a full length or fragment of NTCP. NTCP is well known in the art to bind to, or interact with, PSIP, therefore, serving as the HBV receptor, in some aspects, NTCP is a PSIP binding motif. In some aspects, the NTCP can be essential for binding of the exosome a cell expressing PSIP. Thus, in some aspects, only the region of NTCP responsible for binding to PSIP is present on the exosomes. In some aspects, the NTCP on exosomes is used to target exosomes to a PSIP expressed on human hepatocytes (that have been infected with HBV). In some aspects, the NTCP sequence as shown in FIG. 21 can be used as the PSIP binding motif. In some aspects, the NTCP comprises a sequence 50, 55, 60, 65, 70, 75, 80, 85, 90, 95 or 99% identical to the sequence of FIG. 21 In some aspects, the PSIP binding motif can be a fragment of NTCP, wherein the fragment of NTCP retains PS1P binding function. In some aspects, a fragment of NTCP can be any fragment of the sequence of FIG. 21 retains PSIP binding function.

In some aspects, any of the disclosed exosomes can be a hepatocyte derived exosome, a mesenchymal stem cell (MSC)-derived exosome, a 293T cell-derived exosome, or a liver stellate cell-derived exosome.

In some aspects, the disclosed exosomes can further comprise a marker. For example, a fluorescent protein can be fused to the transmembrane protein on the opposite end from the NTCP binding motif or PSIP binding motif, wherein the fluorescent protein is found on the intracellular side of the exosome. In some aspects, the marker can be any fluorescent protein such as, but not limited to red fluorescent protein, green fluorescent protein or other colors such as blue, orange-red, far-red, cyan, and yellow.

Disclosed are exosomes comprising NTCP binding motif or PSIP binding motif as described herein and further comprising a therapeutic agent. In some aspects, the therapeutic agent can be a peptide, nucleic acid sequence, compound, or combination thereof. In some aspects, the therapeutic agent can be anything that provides a therapeutic effect on a target cell, for example an HBV infected hepatocyte. Thus, in some aspects, a therapeutic agent can be anything that treats an HBV infection, such as, but not limited to, inhibiting HBV replication.

In some aspects, the therapeutic agent is a gRNA/Cas ribonucleoprotein complex comprising a gRNA and a Cas protein. Disclosed are exosomes comprising a NTCP binding motif as described herein and further comprising a gRNA/Cas ribonucleoprotein complex comprising a gRNA and a Cas protein. In some aspects, the gRNA targets, or is specific to, the HBV genome in a cell. In some aspects, the gRNA is specific to a region of the HBV genome comprising the polymerase gene, capsid gene, surface protein genes, or X genes. In some aspects, the gRNA comprises the sequence of one or more of the gRNAs in Table 1, Table 2, or Table 3. In some aspects, the therapeutic agent is a gRNA/Cas ribonucleoprotein complex comprising a gRNA and a Cas protein, wherein the Cas is Cas9, Cas12, or Cas13.

TABLE 1
gRNAs for use with Cas 9

| | |
|---|---|
| HBV gRNA1: | GATTGAGACCTTCGTCTGCGNGG (SEQ |
| | ID NO: 18) |
| HBV gRNA2: | GATTGAGATCTTCTGCGACGNGG (SEQ |
| | ID NO: 19) |
| HBV gRNA3: | GTCGCAGAAGATCTCAATCTNGG (SEQ |
| | ID NO: 20) |
| HBV gRNA4: | GCTCCTACCTTGTTGGCGTCNGG (SEQ |
| | ID NO: 21) |
| HBV gRNA5: | GTTATCGCTGGATGTGTCTGNGG (SEQ |
| | ID NO: 22) |
| HBV gRNA6: | GCTATGCCTCATCTTCTTGTNGG (SEQ |
| | ID NO: 23) |
| HBV gRNA7: | GTCGGAACGGCAGACGGAGANGG |
| | ((SEQ ID NO: 24) |
| HBV gRNA8: | GAAGCGAAGTGCACACGGTCNGG |
| | (SEQ ID NO: 25) |
| HBV gRNA9: | GGTCTCCATGCGACGTGCAGNGG (SEQ |
| | ID NO: 26) |

TABLE 2
gRNAs for use with Cas12

| genomic | |
|---|---|
| location of | |
| ayw strain of | |
| HBV | |
| gRNAseq: | TTTAAATGTATACCCAAAGACAAAAGAAAATTGGTAACAGCGGTA |
| 793 | A (SEQ ID NO: 27) |
| seq: 1750 | TTTATGCCTACAGCCTCCTAGTACAAAGACCTTTAACCTAATCTCC |
| | (SEQ ID NO: 28) |
| seq: 534 | TTTGGTACAGCAACAGGAGGGATACATAGAGGTTCCTTGAGCAGT |
| | A (SEQ ID NO: 29) |
| seq: 1825 | TTTCACCTCTGCCTAATCATCTCTTGTTCATGTCCTACTGTTCAAG |
| | (SEQ ID NO: 30) |
| seq: 2693 | TTTGGAAGTAATGATTAACTAGATGTTCTGGATAATAAGGTTTAAT |
| | (SEQ ID NO: 31) |
| seq: 2230 | TTTGGAAGAGAAACAGTTATAGAGTATTTGGTGTCTTTCGGAGTGT |
| | (SEQ ID NO: 32) |
| seq: 2591 | TTTGTAGGCCCACTCACAGTTAATGAGAAAAGAAGATTGCAATTG |
| | A (SEQ ID NO: 33) |
| seq: 829 | TTTAGAGAGTAACCCCATCTCTTTGTTTTGTTAGGGTTTAAATGTA |
| | (SEQ ID NO: 34) |
| seq: 277 | TTTCTAGGGGGAACTACCGTGTGTCTTGGCCAAAATTCGCAGTCCC |
| | (SEQ ID NO: 35) |
| seq: 265 | TTTGGCCAAGACACACGGTAGTTCCCCCTAGAAAATTGAGAGAAG |
| | T (SEQ ID NO: 36) |
| seq: 2652 | TTTATCCAAAGGTTACCAAATATTTACCATTGGATAAGGGTATTAA |
| | (SEQ ID NO: 37) |
| seq: 2653 | TTTAATACCCTTATCCAATGGTAAATATTTGGTAACCTTTGGATAA |
| | (SEQ ID NO: 38) |

TABLE 3
gRNAs for use with Cas13

| | |
|---|---|
| gRNA 1, | CCCCUACCAACUGGUCGGGGUUUCUGGGAUCUUGCA |
| location 12- | GAGUUUGGUGGAAG (SEQ ID NO: 39) |
| 38 Pre | |
| S2/Polymerase | |
| gRNA 2, | CCCCUACCAACUGGUCGGGGUUUAUUCAGCGCCGAC |
| location | GGGACGUAAACAAA (SEQ ID NO: 40) |
| 1422-1448, | |
| Polymerase/ | |
| HBxAg | |
| gRNA 3, | CCCCUACCAACUGGUCGGGGUUUAUCUUCUGCGACG |
| location | CGGCGAUUGAGACC (SEQ ID NO: 41) |
| 2402-2428, | |
| Core | |
| gRNA 4, | CCCCUACCAACUGGUCGGGGUUUACGCCGCAGACAC |
| location 367- | AUCCAGCGAUAACC (SEQ ID NO: 42) |
| 393, | |
| HBsAg/S/ | |
| Polymerase | |
| gRNA 5, | CCCCUACCAACUGGUCGGGGUUUACGGUGGUCUCCA |
| location | UGCGACGUGCAGAG (SEQ ID NO: 43) |
| 1597-1623, | |
| Polymerase/ | |
| HBxAg | |
| gRNA 6, | CCCCUACCAACUGGUCGGGGUUUCUUUGAAGUAUGC |
| location | CUCAAGGUCGGUCG (SEQ ID NO: 44) |
| 1688-1714, | |
| Enhancer | |
| 2/X/Core | |
| gRNA 7, | CCCCUACCAACUGGUCGGGGUUUUGAUUAGGCAGAG |
| location, | GUGAAAAAGUUGCA (SEQ ID NO: 45) |
| 1816-1842, | |
| PreC/X | |
| gRNA 8, | CCCCUACCAACUGGUCGGGGUUUGACCUGCCUCGUC |
| location, | GUCUAACAACAGUA (SEQ ID NO: 46) |
| 2339-2365, | |
| P/C | |
| gRNA 9, | CCCCUACCAACUGGUCGGGGUUUAUUAACUGUGAGU |
| location, | GGGCCUACAAACUG (SEQ ID NO: 47) |
| 2587-2603, P | |
| gRNA10, | CCCCUACCAACUGGUCGGGGUUUCCC |
| location, | GCCUUCCAUAGAGUGUGUAAAUAG |
| 2745-2771, P | (SEQ ID NO: 48) |

In some aspects, the therapeutic agent is an anti-HBV nucleoside or nucleotide analogue such as, but not limited to, lamivudine, telbivudine, entecavir, adefovir dipivoxil or tenofovir disoproxil fumarate. In some aspects, the therapeutic agent is an anti-HCC (hepatocellular carcinoma) drug, or drug treating non-alcohol fatty liver or other liver diseases. In some aspects, the therapeutic agents can be used to treat liver diseases and can be delivered by our engineered exosomes.

In some aspects, the exosomes have a diameter of about 100 nm. In some aspects, the exosomes have a diameter of about 30-160 nm. In some aspects, the exosomes have a diameter of about 50-120 nm.

In some aspects, the exosomes are derived from human cells. For example, disclosed are human hepatocyte derived exosomes. In some aspects, the hepatocytes are primary human hepatocytes (PHHs). In some aspects, the exosomes can be derived from hepatocyte cell lines. For example, hepatocyte cell lines can be, but are not limited to, Hep G2 or Hue-7.

### C. Nucleic Acid Sequences

### 1. Guide RNAs

Disclosed are nucleic acid sequences of the gRNAs used in CRISPR for targeting the HBV genome. Disclosed are nucleic acid sequences of the gRNAs used in a gRNA/Cas ribonucleoprotein complex.

Disclosed are gRNAs specific to a region of the HBV genome comprising the polymerase gene, capsid gene, surface protein genes, or X genes. In some aspects, the gRNA comprises one or more of the sequences of Table 1-3.

In some aspects, the gRNAs can be designed using the online tool CHOPCHOP (//chopchop.cbu.uib.no/) (Labun et al.CHOPCHOP v3: expanding the CRISPR web toolbox beyond genome editing. Nucleic Acids Research (2019)) based on the ayw strain of HBV (Genbank accession number: NC_003977.2).

In some aspects, the disclosed gRNAs can be complexed with Cas. For example, disclosed are gRNA/Cas ribonucleoprotein complexes comprising a gRNA and a Cas protein, wherein the gRNA comprises one or more of the sequences of Tables 1, 2 or 3. In some aspects, the Cas protein can be Cas9, Cas12, or Cas13.

In some aspects, the gRNA in the gRNA/Cas ribonucleoprotein complexes can be any of those described herein.

### 2. Nucleic Acid Constructs

Disclosed are nucleic acid constructs comprising a nucleic acid sequence capable of encoding a fusion protein, wherein the fusion protein comprises a transmembrane protein and a NTCP binding motif. In some aspects, disclosed are nucleic acid constructs comprising a nucleic acid sequence capable of encoding a fusion protein, wherein the fusion protein comprises a marker protein, a transmembrane protein and a NTCP binding motif. In some aspects, the NTCP binding motif is PSIP. In some aspects, the NTCP binding motif is amino acids 2-47 of PSIP. In some aspects, the nucleic acid sequence that encodes amino acids 2-47 of PSIP is a top strand of double stranded DNA having the sequence of AACTCTAGAATGGGACAGAATCTTTCTGTGCCCAATCCT CTGGGATTCTTTCCAGACCACCAGTTGGATCCACTATTCAGAGCAAATTC CAGCAGTCCC GATTGGGACTTCAACACAAACAAGGACAATTGGCCAATGG CAAACAAGGTA (SEQ ID NO: 16) and a bottom strand of double stranded DNA having the sequence of

(SEQ ID NO: 17)
TGGAATTCTACCTTGTTTGCCATTGGCCAATTGTCCTTGTTTGTGTTGAAG
TCCCAATCGGGACTGCTGGAATTTGCTCTGAATAGTGGATCCAACTGGTGG
TCTGGAAAGAATCCCAGAGGATTGGGCACAGAAAGATTCTGTCCCAT.

In some aspects, the transmembrane protein is CD9. In some aspects, CD9 can be truncated. For example, amino acids 1-37 of CD9 can be deleted. In some aspects, the transmembrane protein can be, but is not limited to, CD63 and CD81.

In some aspects, the marker can be a fluorescent protein such as, but not limited to, red, green, blue, orange-red, far-red, cyan, and yellow fluorescent protein. For example, the fusion protein can be red fluorescent protein-CD9-NTCP binding motif.

Also disclosed are nucleic acid constructs comprising a nucleic acid sequence comprising a promoter operably linked to one or more of the disclosed nucleic acid constructs. For example, disclosed are nucleic acid constructs comprising a nucleic acid sequence comprising a promoter operably linked to a nucleic acid sequence capable of encoding a fusion protein, wherein the fusion protein comprises a transmembrane protein and a NTCP binding motif.

### 3. Vectors

Disclosed are vectors comprising any of the nucleic acid constructs disclosed herein.

The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vector can be a viral vector. For example, the viral vector can be an adeno-associated viral vector. In some aspects, the vector can be a non-viral vector, such as a DNA based vector.

i. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding a fusion protein, wherein the fusion protein comprises a marker protein, a transmembrane protein and a NTCP binding motif.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORTI plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes β-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209:1422 (1980)) or hygromycin, (Sugden, B. et al., Mol. Cell. Biol. 5:410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I.M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### D. Compositions

Disclosed are compositions comprising any of the disclosed gRNAs, gRNA/Cas ribonucleoprotein complex, exosomes, or combinations thereof.

In some instances, the compositions can further comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG: PC: Cholesterol: peptide or PC: peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed gRNAs, gRNA/Cas ribonucleoprotein complex, exosomes, or combinations thereof can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. peptide) in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the gRNAs, gRNA/Cas ribonucleoprotein complex, exosomes, or combinations thereof disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising any of the disclosed gRNAs, gRNA/Cas ribonucleoprotein complex, exosomes, or combinations thereof described herein, or a pharmaceutically acceptable salt or solvate thereof, and a pharmaceutically acceptable carrier, buffer, or diluent. In various aspects, the gRNAs, gRNA/Cas ribonucleoprotein complex, exosomes, or combinations thereof of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the peptides described herein and can also include a carrier such as a pharmaceutically acceptable carrier. For example, disclosed are pharmaceutical compositions, comprising the peptides disclosed herein, and a pharmaceutically acceptable carrier. In one aspect, disclosed are pharmaceutical compositions comprising the disclosed gRNAs, gRNA/Cas ribonucleoprotein complex, exosomes, or combinations thereof. That is, a pharmaceutical composition can be provided comprising a therapeutically effective amount of at least one disclosed gRNAs, gRNA/Cas ribonucleoprotein complex, exosomes, or combinations thereof or at least one product of a disclosed method and a pharmaceutically acceptable carrier.

In certain aspects, the disclosed pharmaceutical compositions comprise the disclosed gRNAs, gRNA/Cas ribonucleoprotein complex, exosomes, or combinations thereof (including pharmaceutically acceptable salt(s) thereof) as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the gRNAs, gRNA/Cas ribonucleoprotein complex, exosomes, or combinations thereof described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

By “pharmaceutically acceptable” is meant a material or carrier that would be selected to minimize any degradation of the active ingredient and to minimize any adverse side effects in the subject, as would be well known to one of skill in the art. The gRNAs, gRNA/Cas ribonucleoprotein complex, exosomes, or combinations thereof described herein, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG: PC: Cholesterol: peptide or PC: peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed peptides in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the ease in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a disclosed peptide (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed exosome, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### E. Methods of Making

Disclosed are methods of making exosomes that have been engineered to express a heterologous binding motif on the surface of the exosome. In some aspects, the heterologous binding motif is expressed as a fusion protein with a transmembrane domain. In some aspects, the heterologous binding motif is expressed as a fusion protein with a transmembrane domain and a marker. Disclosed are methods of making exosomes comprising a NTCP binding motif. Disclosed are methods of making exosomes comprising a NTCP binding motif fused to a transmembrane protein wherein the transmembrane protein is CD9, CD63 or CD81. Disclosed are methods of making exosomes comprising a NTCP binding motif fused to a transmembrane protein wherein the transmembrane protein is a truncated CD9.

Disclosed are methods of making hepatocyte-derived exosomes comprising a NTCP binding motif. Disclosed are methods of making hepatocyte-derived exosomes comprising a NTCP binding motif fused to a transmembrane protein wherein the transmembrane protein is CD9, CD63 or CD81. Disclosed are methods of making hepatocyte-derived exosomes comprising a NTCP binding motif fused to a transmembrane protein wherein the transmembrane protein is a truncated CD9.

Disclosed are methods of making an engineered cell (e.g. hepatocyte) derived exosome comprising transfecting a plasmid into a cell (e.g. hepatocyte), wherein the plasmid comprises a nucleic acid sequence capable of encoding a fusion protein, wherein the fusion protein comprises a transmembrane protein and an extracellular target-specific binding motif; culturing the cells (e.g. hepatocytes) to allow production of exosomes expressing the transmembrane protein and an extracellular target-specific binding motif fusion protein; and obtaining an exosome-containing supernatant. In some aspects, the cells are hepatocytes, MSCs, 293T cells, or liver stellate cells.

In some aspects, the extracellular target-specific binding motif can be a NTCP binding motif. Disclosed are methods of making an engineered cell (e.g. hepatocyte) derived exosome comprising transfecting a plasmid into cells (e.g. hepatocytes), wherein the plasmid comprises a nucleic acid sequence capable of encoding a fusion protein, wherein the fusion protein comprises a transmembrane protein and a NTCP binding motif, culturing the cells (e.g. hepatocytes) to allow production of exosomes expressing the fusion protein; and obtaining an exosome-containing supernatant. In some aspects, the cells are hepatocytes, MSCs, 293T cells, or liver stellate cells.

In some aspects, the transmembrane can be CD9, CD81, or CD63. Disclosed are methods of making an engineered cell derived exosome (e.g. hepatocyte derived exosome) comprising transfecting a plasmid into cells (e.g. hepatocytes), wherein the plasmid comprises a nucleic acid sequence capable of encoding a fusion protein, wherein the fusion protein comprises CD9 and a target-specific (e.g. NTCP) binding motif, culturing the cells (e.g. hepatocytes) to allow production of exosomes expressing the fusion protein; and obtaining an exosome-containing supernatant. In some aspects, the cells are hepatocytes, MSCs, 293T cells, or liver stellate cells.

In some aspects, the fusion protein can further comprise a marker. Disclosed are methods of making an engineered cell derived exosome (e.g. hepatocyte derived exosome) comprising transfecting a plasmid into cells (e.g. hepatocytes), wherein the plasmid comprises a nucleic acid sequence capable of encoding a fusion protein, wherein the fusion protein comprises a marker, a transmembrane protein (e.g. CD9), and a target-specific binding motif (e.g. NTCP), culturing the cells (e.g. hepatocytes) to allow production of exosomes expressing the fusion protein; and obtaining an exosome-containing supernatant. In some aspects, the marker can be a fluorescent protein such as, but not limited to, red, green, blue, orange-red, far-red, cyan, and yellow fluorescent protein. For example, the fusion protein can be red fluorescent protein-transmembrane protein-target specific binding motif. In some aspects, the cells are hepatocytes, MSCs, 293T cells, or liver stellate cells.

In some aspects of the disclosed methods, as part of the culturing, a screening step can be performed. Thus, in some aspects, the disclosed methods can further comprise screening for cells (e.g. hepatocytes) producing exosomes expressing the transmembrane protein and a target-specific binding motif fusion protein. In some aspects, the fusion protein further comprises a marker to allow for screening. For example, a fluorescent protein expressed in the cells can be identified and thus used to screen for cells that comprise the plasmid.

In some aspects, obtaining an exosome-containing supernatant comprises centrifuging the cells (e.g. hepatocytes) and collecting the exosome-containing supernatant. In some aspects, the exosome-containing supernatant is collected 48-72 hours after transfecting. In some aspects, the exosome-containing supernatant is collected at least 12, 18, 24, 30, 36, 42, 48, 54, 60, 66, or 72 hours after transfecting. In some aspects, all cells produce exosomes thus providing sufficient time for the transfected plasmid to express the fusion protein results in exosomes expressing the fusion protein.

In some aspects, the disclosed methods can further comprise isolating the engineered cell (e.g. hepatocyte) derived exosomes from the exosome-containing supernatant. In some aspects, isolating the engineered cell (e.g. hepatocyte) derived exosomes from the exosome-containing supernatant comprises ultra-centrifuging the exosome-containing supernatant to obtain the engineered cell (e.g. hepatocyte) derived exosomes and or purifying the engineered cell (e.g. hepatocyte) derived exosomes from exosome-containing supernatant using column chromatography.

In some aspects, the exosomes have a diameter of about 100 nm. In some aspects, the exosomes have a diameter of about 30-160 nm. In some aspects, the exosomes have a diameter of about 50-120 nm.

In some aspects, the disclosed methods further comprise loading the cell (e.g. hepatocyte) derived exosomes with a therapeutic agent. In some aspects, loading comprises electroporation. Electroporation techniques are well known in the art for allowing an agent (e.g. therapeutic) to enter a cell. In some aspects, loading comprises the use of transfection reagents to get the therapeutic agent inside the cell.

In some aspects, the therapeutic agent is a gRNA/Cas ribonucleoprotein complex comprising a gRNA and a Cas protein. In some aspects, the gRNA targets, or is specific to, the HBV genome in a cell, thus the gRNAs can be referred to as HBV specific gRNAs. a region of the HBV genome comprising the polymerase gene, capsid gene, surface protein genes, or X genes. In some aspects, the gRNA comprises the sequence of one or more of the gRNAs in Tables 1, 2, or 3. In some aspects, the therapeutic agent is a gRNA/Cas ribonucleoprotein complex comprising a gRNA and a Cas protein, wherein the Cas is Cas9, Cas12, or Cas13.

In some aspects, the therapeutic agent is the therapeutic agent is an anti-HBV nucleoside or nucleotide analogue such as, but not limited to, lamivudine, telbivudine, entecavir, adefovir dipivoxil or tenofovir disoproxil fumarate.

In some aspects, the cells used to make the exosomes are human cells. For example, human hepatoctyes can be transfected with the plasmid. Thus, in some aspects, the exosomes produced are human cell derived exosomes. In some aspects, the hepatocytes are primary human hepatocytes (PHHs). In some aspects, the exosomes can be derived from hepatocyte cell lines. For example, hepatocyte cell lines can be, but are not limited to, Hep G2 or Hun-7.

### F. Methods of Use

### 1. Methods of Treating

Disclosed are methods of treating a subject having a liver disease comprising administering to the subject a therapeutically effective amount of an exosome, wherein the exosome comprises a NTCP binding motif and a therapeutic agent, thereby treating the liver disease in the subject. In some aspects, the liver disease can be a hepatitis B virus (HBV) infection, non-alcohol fatty liver, genetic liver disease, and hepatocellular carcinoma (HCC).

Disclosed are methods of treating a subject infected with hepatitis B virus (HBV) comprising administering to the subject a therapeutically effective amount of an exosome, wherein the exosome comprises a NTCP binding motif or PSIP binding motif and a therapeutic agent, thereby treating the HBV infection in the subject.

Disclosed are methods of treating a subject infected with HBV or having any liver disease comprising administering to the subject a therapeutically effective amount of hepatocyte-derived exosomes, wherein the hepatocyte-derived exosomes comprise a NTCP binding motif and a therapeutic agent, thereby treating the HBV infection or liver disease in the subject. In some aspects, the use of a NTCP binding motif allows for targeting of the exosome to a cell expressing NTCP, such as a hepatocyte. Thus, in some aspects, the NTCP binding motif targets the hepatocyte-derived exosome to a hepatocyte and the therapeutic agent in the exosome can provide therapeutic effects on HBV present in the hepatocyte.

In some aspects, the exosomes comprising an NTCP binding motif, such as PSIP, can target additional HBV receptors expressed on cells infected by HBV. In some aspects, any other cell that can be infected by HBV must have a receptor (e.g. other than NTCP) for HBV on the cell surface, thus the PSIP from the exosomes can bind to the HBV receptor and allow the exosome to deliver the therapeutic agent.

In some aspects, the therapeutic agent is inside (e.g. packaged inside) of the exosome (e.g. hepatocyte-derived exosome). In some aspects, the therapeutic agent is present on the outside of the exosome (e.g. hepatocyte-derived exosome).

In some aspects, the therapeutic agent is a gRNA/Cas ribonucleoprotein complex comprising a gRNA and a Cas protein. In some aspects, the gRNA targets, or is specific to, the HBV genome in a cell, thus the gRNAs can be referred to as HBV specific gRNAs. a region of the HBV genome comprising the polymerase gene, capsid gene, surface protein genes, or X genes. In some aspects, the gRNA comprises the sequence of one or more of the gRNAs in Tables 1, 2, or 3. In some aspects, the therapeutic agent is a gRNA/Cas ribonucleoprotein complex comprising a gRNA and a Cas protein, wherein the Cas is Cas9, Cas12, or Cas13.

In some aspects, the gRNA/Cas ribonucleoprotein cleaves HBV genomic DNA (and/or RNA) in an HBV infected cell of the subject. In some aspects, the gRNA/Cas ribonucleoprotein cleaves covalently closed circular DNA (cccDNA) of HBV as well as integrated HBV DNA.

In some aspects, the therapeutic agent is an anti-HBV nucleoside or nucleotide analogue such as, but not limited to, lamivudine, telbivudine, entecavir, adefovir dipivoxil or tenofovir disoproxil fumarate. In some aspects, these therapeutic agents target and inhibit the HBV DNA polymerase.

In some aspects, the NTCP binding motif is fused to a transmembrane domain. In some aspects, the fusion of the NTCP binding motif with a transmembrane domain allows for the transmembrane domain to position the NTCP binding motif on the external surface of the exosome thus exposed to a binding partner of the NTCP binding motif (e.g. NTCP). In some aspects, the transmembrane protein is CD9, CD63 or CD81. In some aspects, disclosed are exosomes comprising a NTCP binding motif, wherein the NTCP binding motif is fused to a truncated CD9 sequence. For example, a truncated CD9 sequence can comprise a CD9 amino acid sequence wherein amino acids 1-37 of CD9 are deleted so that PSIP is present on the surface of exosome.

In some aspects, a therapeutically effective amount of hepatocyte derived exosomes comprises about 1×10⁶ to 1×10¹⁰ particles. In some aspects, a therapeutically effective amount of hepatocyte derived exosomes comprises about 1×10⁸. In some aspects, a therapeutically effective amount of hepatocyte derived exosomes are packaged with about 40 μM each of gRNA RNPs.

In some aspects, administering can be intravenous administration. In some aspects, administering can be intramuscular, intra-liver, subcutaneous, intraperitoneal, oral, or nasal. Thus, in some aspects, the therapeutically effective amount of cell (e.g. hepatocyte) derived exosomes is present in a composition, such as a pharmaceutically acceptable composition, formulated for the specific delivery type as described herein.

In some aspects, any of the exosomes or compositions disclosed herein can be used in the disclosed methods.

The disclosed methods of treating HBV infection can be performed using an exosome comprising a PSP binding motif. Thus, the exosomes would target a cell expressing HBV surface antigen (HBsAg) including preS1 peptide (PSIP) and the NTCP (PSIP binding motif) on the exosome

### 2. Methods of Inactivating HBV DNA

Disclosed are methods of inactivating HBV DNA in HBV infected cells comprising administering to the HBV infected cells a therapeutically effective amount of exosomes, wherein the exosomes comprise a NTCP binding motif or PS1P binding motif and a therapeutic agent, thereby inactivating HBV DNA in the HBV infected cells. In some aspects, the HBV DNA in HBV infected cells is cccDNA or integrated DNA (iDNA).

Disclosed are methods of inactivating HBV cccDNA or iDNA in HBV infected cells comprising administering to the HBV infected cells a therapeutically effective amount of hepatocyte derived exosomes, wherein the hepatocyte derived exosomes comprise a NTCP binding motif or PSIP binding motif and a therapeutic agent, thereby inactivating HBV cccDNA or iDNA in the HBV infected cells. In some aspects, the use of a NTCP binding motif or PSIP binding motif allows for targeting of the exosome to hepatocytes, and HBV infects hepatocytes. Thus, in some aspects, the NTCP binding motif targets the hepatocyte-derived exosome to hepatocytes, which can be infected with HBV, and the therapeutic agent in the exosome can provide therapeutic effects on the HBV in the hepatocyte resulting in less virus production (e.g. inactivation of cccDNA or iDNA). In some aspects, the PSIP binding motif targets the hepatocyte-derived exosome to hepatocytes infected with HBV and expressing HBsAg including PSIP, and the therapeutic agent in the exosome can provide therapeutic effects on the HBV in the hepatocyte resulting in less virus production (e.g. excision and/or inactivation of cccDNA or iDNA).

In some aspects, the hepatocytes are in vitro. In some aspects, the hepatocytes are in a subject.

In some aspects, the therapeutic agent is inside (e.g. packaged inside) of the exosome (e.g. hepatocyte-derived exosome). In some aspects, the therapeutic agent is present on the outside of the exosome (e.g. hepatocyte-derived exosome).

In some aspects, the therapeutic agent is a gRNA/Cas ribonucleoprotein complex comprising a gRNA and a Cas protein. In some aspects, the gRNA targets, or is specific to, the HBV genome in a cell, thus the gRNAs can be referred to as HBV specific gRNAs. a region of the HBV genome comprising the polymerase gene, capsid gene, surface protein genes, or X genes. In some aspects, the gRNA comprises the sequence of one or more of the gRNAs in Tables 1, 2, or 3. In some aspects, the therapeutic agent is a gRNA/Cas ribonucleoprotein complex comprising a gRNA and a Cas protein, wherein the Cas is Cas9, Cas12, or Cas13.

In some aspects, the gRNA/Cas ribonucleoprotein cleaves HBV genomic DNA (and/or RNA) in an HBV infected cell of the subject. In some aspects, the gRNA/Cas ribonucleoprotein cleaves HBV covalently closed circular DNA (cccDNA) and/or HBV integrated DNA (iDNA).

In some aspects, the therapeutic agent is an anti-HBV nucleoside or nucleotide analogue such as, but not limited to, lamivudine, telbivudine, entecavir, adefovir dipivoxil or tenofovir disoproxil fumarate. In some aspects, these therapeutic agents target and inhibit the HBV DNA polymerase.

In some aspects, the NTCP binding motif is fused to a transmembrane domain. In some aspects, the fusion of the NTCP binding motif with a transmembrane domain allows for the transmembrane domain to position the NTCP binding motif on the external surface of the exosome thus exposed to a binding partner of the NTCP binding motif (e.g. NTCP). In some aspects, the transmembrane protein is CD9, CD63 or CD81. In some aspects, disclosed are exosomes comprising a NTCP binding motif, wherein the NTCP binding motif is fused to a truncated CD9 sequence. For example, a truncated CD9 sequence can comprise a CD9 amino acid sequence wherein amino acids 1-37 of CD9 are deleted.

In some aspects, a therapeutically effective amount of hepatocyte derived exosomes comprises about 1×10⁶ to 1×10¹⁰ particles. In some aspects, a therapeutically effective amount of hepatocyte derived exosomes comprises about 1×10⁸. In some aspects, a therapeutically effective amount of hepatocyte derived exosomes are packaged with about 40 μM each of gRNA RNPs.

In some aspects, administering can be intravenous administration. In some aspects, administering can be intramuscular, intra-liver, subcutaneous, intraperitoneal, oral, or nasal. Thus, in some aspects, the therapeutically effective amount of cell (e.g. hepatocyte) derived exosomes is present in a composition, such as a pharmaceutically acceptable composition, formulated for the specific delivery type as described herein.

In some aspects, any of the exosomes or compositions disclosed herein can be used in the disclosed methods.

### 3. Methods of Delivering a Therapeutic Agent to HBV Infected Cells

Disclosed are methods of delivering a therapeutic agent to HBV target cells comprising administering to the HBV target cells a therapeutically effective amount of exosomes, wherein the exosomes comprise a NTCP binding motif and a therapeutic agent. In some aspects, delivering a therapeutic agent to HBV target cells can also be referred to as targeting a therapeutic agent to HBV target cells. In some aspects, HBV target cells are hepatocytes since HBV is known to target and infect hepatocytes. Thus, disclosed are methods of delivering a therapeutic agent to hepatocytes comprising administering to the hepatocytes a therapeutically effective amount of exosomes, wherein the exosomes comprise a NTCP binding motif and a therapeutic agent. Because HBV infects hepatocytes, also disclosed are methods of delivering a therapeutic agent to HBV infected cells comprising administering to the HBV infected cells a therapeutically effective amount of exosomes, wherein the exosomes comprise a NTCP binding motif and a therapeutic agent.

In some aspects, the HBV target cells, hepatocytes, and/or HBV infected cells are in vitro. In some aspects, the HBV target cells, hepatocytes, and/or HBV infected cells are in a subject. Thus, in some aspects, delivering a therapeutic agent to HBV target cells, hepatocytes, and/or HBV infected other types of cells can include delivering a therapeutic agent to a subject infected with HBV.

In some aspects, the therapeutic agent is inside (e.g. packaged inside) of the exosome (e.g. hepatocyte-derived exosome). In some aspects, the therapeutic agent is present on the outside of the exosome (e.g. hepatocyte-derived exosome).

In some aspects, the therapeutic agent is a gRNA/Cas ribonucleoprotein complex comprising a gRNA and a Cas protein. In some aspects, the gRNA targets, or is specific to, the HBV genome in a cell, thus the gRNAs can be referred to as HBV specific gRNAs. a region of the HBV genome comprising the polymerase gene, capsid gene, surface protein genes, or X genes. In some aspects, the gRNA comprises the sequence of one or more of the gRNAs in Tables 1, 2, or 3. In some aspects, the therapeutic agent is a gRNA/Cas ribonucleoprotein complex comprising a gRNA and a Cas protein, wherein the Cas is Cas9, Cas12, or Cas13.

In some aspects, the gRNA/Cas ribonucleoprotein cleaves HBV genomic DNA (and/or RNA) in an HBV infected cell of the subject. In some aspects, the gRNA/cas ribonucleoprotein cleaves HBV covalently closed circular DNA (cccDNA) and/or HBV integrated DNA (iDNA).

In some aspects, the therapeutic agent is an anti-HBV nucleoside or nucleotide analogue such as, but not limited to, lamivudine, telbivudine, entecavir, adefovir dipivoxil or tenofovir disoproxil fumarate. In some aspects, these therapeutic agents target and inhibit the HBV DNA polymerase.

In some aspects, the NTCP binding motif is fused to a transmembrane domain. In some aspects, the fusion of the NTCP binding motif with a transmembrane domain allows for the transmembrane domain to position the NTCP binding motif on the external surface of the exosome thus exposed to a binding partner of the NTCP binding motif (e.g. NTCP). In some aspects, the transmembrane protein is CD9, CD63 or CD81. In some aspects, disclosed are exosomes comprising a NTCP binding motif, wherein the NTCP binding motif is fused to a truncated CD9 sequence. For example, a truncated CD9 sequence can comprise a CD9 amino acid sequence wherein amino acids 1-37 of CD9 are deleted.

In some aspects, a therapeutically effective amount of hepatocyte derived exosomes comprises about 1×10⁶ to 1×10¹⁰ particles. In some aspects, a therapeutically effective amount of hepatocyte derived exosomes comprises about 1×10⁸. In some aspects, a therapeutically effective amount of hepatocyte derived exosomes are packaged with about 40 μM each of gRNA RNPs.

In some aspects, administering can be intravenous administration. In some aspects, administering can be intramuscular, intra-liver, subcutaneous, intraperitoneal, oral, or nasal. Thus, in some aspects, the therapeutically effective amount of cell (e.g. hepatocyte) derived exosomes is present in a composition, such as a pharmaceutically acceptable composition, formulated for the specific delivery type as described herein.

In some aspects, any of the exosomes or compositions disclosed herein can be used in the disclosed methods.

### 4. Methods of Reducing HBV Production

Disclosed are methods of reducing HBV production from cells infected with HBV comprising administering to the HBV infected cells a therapeutically effective amount of exosomes, wherein the exosomes comprise a NTCP binding motif and a therapeutic agent, thereby reducing HBV production in the cells infected with HBV.

Disclosed are methods of reducing HBV production from cells infected with HBV comprising administering to the HBV infected cells a therapeutically effective amount of hepatocyte derived exosomes, wherein the hepatocyte derived exosomes comprise a NTCP binding motif and a therapeutic agent, thereby reducing HBV production in the cells infected with HBV. In some aspects, the use of a NTCP binding motif allows for targeting of the exosome to a cell expressing NTCP, such as hepatocytes. Thus, in some aspects, the NTCP binding motif targets the hepatocyte-derived exosome to a hepatocyte, which can be infected with HBV, and the therapeutic agent in the exosome can provide therapeutic effects on the HBV in the hepatocyte resulting in less virus production.

In some aspects, the hepatocytes are in vitro. In some aspects, the hepatocytes are in a subject.

In some aspects, the therapeutic agent is inside (e.g. packaged inside) of the exosome (e.g. hepatocyte-derived exosome). In some aspects, the therapeutic agent is present on the outside of the exosome (e.g. hepatocyte-derived exosome).

In some aspects, the therapeutic agent is a gRNA/Cas ribonucleoprotein complex comprising a gRNA and a Cas protein. In some aspects, the gRNA targets, or is specific to, the HBV genome in a cell, thus the gRNAs can be referred to as HBV specific gRNAs. a region of the HBV genome comprising the polymerase gene, capsid gene, surface protein genes, or X genes. In some aspects, the gRNA comprises the sequence of one or more of the gRNAs in Tables 1, 2, or 3. In some aspects, the therapeutic agent is a gRNA/Cas ribonucleoprotein complex comprising a gRNA and a Cas protein, wherein the Cas is Cas9, Cas12, or Cas13.

In some aspects, the gRNA/Cas ribonucleoprotein cleaves HBV genomic DNA (and/or RNA) in an HBV infected cell of the subject. In some aspects, the gRNA/cas ribonucleoprotein cleaves HBV covalently closed circular DNA (cccDNA) and/or HBV integrated DNA (iDNA).

In some aspects, the therapeutic agent is an anti-HBV nucleoside or nucleotide analogue such as, but not limited to, lamivudine, telbivudine, entecavir, adefovir dipivoxil or tenofovir disoproxil fumarate. In some aspects, these therapeutic agents target and inhibit the HBV DNA polymerase.

In some aspects, the NTCP binding motif is fused to a transmembrane domain. In some aspects, the fusion of the NTCP binding motif with a transmembrane domain allows for the transmembrane domain to position the NTCP binding motif on the external surface of the exosome thus exposed to a binding partner of the NTCP binding motif (e.g. NTCP). In some aspects, the transmembrane protein is CD9, CD63 or CD81. In some aspects, disclosed are exosomes comprising a NTCP binding motif, wherein the NTCP binding motif is fused to a truncated CD9 sequence. For example, a truncated CD9 sequence can comprise a CD9 amino acid sequence wherein amino acids 1-37 of CD9 are deleted.

In some aspects, a therapeutically effective amount of hepatocyte derived exosomes comprises about 1×10⁶ to 1×10¹⁰ particles. In some aspects, a therapeutically effective amount of hepatocyte derived exosomes comprises about 1×10⁸. In some aspects, a therapeutically effective amount of hepatocyte derived exosomes are packaged with about 40 μM each of gRNA RNPs.

In some aspects, administering can be intravenous administration. In some aspects, administering can be intramuscular, intra-liver, subcutaneous, intraperitoneal, oral, or nasal. Thus, in some aspects, the therapeutically effective amount of cell (e.g. hepatocyte) derived exosomes is present in a composition, such as a pharmaceutically acceptable composition, formulated for the specific delivery type as described herein.

In some aspects, any of the exosomes or compositions disclosed herein can be used in the disclosed methods.

### 5. Methods of Cleaving a HBV Genome

Disclosed are methods of cleaving a HBV genome in a cell infected with HBV comprising administering to the HBV infected cells a therapeutically effective amount of exosomes, wherein the exosomes comprise a NTCP binding motif and a therapeutic agent, wherein the therapeutic agent is a HBV specific gRNA/Cas ribonucleoprotein complex comprising a HBV specific gRNA and a Cas protein.

Disclosed are methods of cleaving a HBV genome in a cell infected with HBV comprising administering to the HBV infected cells a therapeutically effective amount of hepatocyte derived exosomes, wherein the hepatocyte derived exosomes comprise a NTCP binding motif and a therapeutic agent, wherein the therapeutic agent is a HBV specific gRNA/Cas ribonucleoprotein complex comprising a HBV specific gRNA and a Cas protein. In some aspects, the use of a NTCP binding motif allows for targeting of the exosome to a cell expressing NTCP, such as hepatocytes. Thus, in some aspects, the NTCP binding motif targets the hepatocyte-derived exosome to a hepatocyte and the gRNA/Cas ribonucleoprotein complex targets and cleaves the HBV genome in the cell.

In some aspects, the HBV infected cells (e.g. hepatocytes) are in vitro. In some aspects, the HBV infected cells (e.g. hepatocytes) are in a subject.

In some aspects, the therapeutic agent is inside (e.g. packaged inside) of the exosome (e.g. hepatocyte-derived exosome). In some aspects, the therapeutic agent is present on the outside of the exosome (e.g. hepatocyte-derived exosome).

In some aspects, the gRNA targets, or is specific to, the HBV genome in a HBV infected cell, thus the gRNAs can be referred to as HBV specific gRNAs. In some aspects, the gRNA is a region of the HBV genome comprising the polymerase gene, capsid gene, surface protein genes, or X genes. In some aspects, the gRNA comprises the sequence of one or more of the gRNAs in Tables 1, 2, or 3. In some aspects, the therapeutic agent is a gRNA/Cas ribonucleoprotein complex comprising a gRNA and a Cas protein, wherein the Cas is Cas9, Cas12, or Cas13.

In some aspects, the gRNA/Cas ribonucleoprotein cleaves HBV genomic DNA (and/or RNA) in an HBV infected cell of the subject. In some aspects, the gRNA/Cas ribonucleoprotein cleaves covalently closed circular DNA (cccDNA) of HBV or HBV integrated DNA (iDNA).

In some aspects, the NTCP binding motif is fused to a transmembrane domain. In some aspects, the fusion of the NTCP binding motif with a transmembrane domain allows for the transmembrane domain to position the NTCP binding motif on the external surface of the exosome thus exposed to a binding partner of the NTCP binding motif (e.g. NTCP). In some aspects, the transmembrane protein is CD9, CD63 or CD81. In some aspects, disclosed are exosomes comprising a NTCP binding motif, wherein the NTCP binding motif is fused to a truncated CD9 sequence. For example, a truncated CD9 sequence can comprise a CD9 amino acid sequence wherein amino acids 1-37 of CD9 are deleted.

In some aspects, a therapeutically effective amount of hepatocyte derived exosomes comprises about 1×10⁶ to 1×10¹⁰ particles. In some aspects, a therapeutically effective amount of hepatocyte derived exosomes comprises about 1×10⁸. In some aspects, a therapeutically effective amount of hepatocyte derived exosomes are packaged with about 40 μM each of gRNA RNPs.

In some aspects, administering can be intravenous administration. In some aspects, administering can be intramuscular, intra-liver, subcutaneous, intraperitoneal, oral, or nasal. Thus, in some aspects, the therapeutically effective amount of cell (e.g. hepatocyte) derived exosomes is present in a composition, such as a pharmaceutically acceptable composition, formulated for the specific delivery type as described herein.

In some aspects, any of the exosomes or compositions disclosed herein can be used in the disclosed methods.

### 6. Methods of Inhibiting HBV Replication

Disclosed are methods of inhibiting HBV replication in an HBV infected cell comprising administering to the HBV infected cells a therapeutically effective amount of exosomes, wherein the exosomes comprise a NTCP binding motif and a therapeutic agent, thereby inhibiting HBV reactivation or replication in the cell.

Disclosed are methods of inhibiting HBV reactivation or replication in an HBV infected cell comprising administering to the HBV infected cells a therapeutically effective amount of hepatocyte-derived exosomes, wherein the hepatocyte-derived exosomes comprise a NTCP binding motif and a therapeutic agent, thereby inhibiting HBV reactivation or replication in the cell. In some aspects, the use of a NTCP binding motif allows for targeting of the exosome to a cell expressing NTCP, such as a hepatocyte. Thus, in some aspects, the NTCP binding motif targets the hepatocyte-derived exosome to a hepatocyte, which can be infected with HBV, and the therapeutic agent in the exosome can provide therapeutic effects on the HBV in the hepatocyte resulting in inhibiting HBV reactivation or replication

In some aspects, the HBV infected cells are in vitro. In some aspects, the HBV infected cells are in a subject.

In some aspects, the therapeutic agent is inside (e.g. packaged inside) of the exosome (e.g. hepatocyte-derived exosome). In some aspects, the therapeutic agent is present on the outside of the exosome (e.g. hepatocyte-derived exosome).

In some aspects, the therapeutic agent is a gRNA/Cas ribonucleoprotein complex comprising a gRNA and a Cas protein. In some aspects, the gRNA targets, or is specific to, the HBV genome in a cell, thus the gRNAs can be referred to as HBV specific gRNAs. a region of the HBV genome comprising the polymerase gene, capsid gene, surface protein genes, or X genes. In some aspects, the gRNA comprises the sequence of one or more of the gRNAs in Tables 1, 2, or 3. In some aspects, the therapeutic agent is a gRNA/Cas ribonucleoprotein complex comprising a gRNA and a Cas protein, wherein the Cas is Cas9, Cas12, or Cas13.

In some aspects, the gRNA/Cas ribonucleoprotein cleaves HBV genomic DNA (and/or RNA) in an HBV infected cell of the subject. In some aspects, the gRNA/cas ribonucleoprotein cleaves HBV covalently closed circular DNA (cccDNA) and/or HBV integrated DNA (iDNA).

In some aspects, the therapeutic agent is an anti-HBV nucleoside or nucleotide analogue such as, but not limited to, lamivudine, telbivudine, entecavir, adefovir dipivoxil or tenofovir disoproxil fumarate. In some aspects, these therapeutic agents target and inhibit the HBV DNA polymerase.

In some aspects, the NTCP binding motif is fused to a transmembrane domain. In some aspects, the fusion of the NTCP binding motif with a transmembrane domain allows for the transmembrane domain to position the NTCP binding motif on the external surface of the exosome thus exposed to a binding partner of the NTCP binding motif (e.g. NTCP). In some aspects, the transmembrane protein is CD9, CD63 or CD81. In some aspects, disclosed are exosomes comprising a NTCP binding motif, wherein the NTCP binding motif is fused to a truncated CD9 sequence. For example, a truncated CD9 sequence can comprise a CD9 amino acid sequence wherein amino acids 1-37 of CD9 are deleted.

In some aspects, a therapeutically effective amount of hepatocyte derived exosomes comprises about 1×10⁶ to 1×10¹⁰ particles. In some aspects, a therapeutically effective amount of hepatocyte derived exosomes comprises about 1×10⁸. In some aspects, a therapeutically effective amount of hepatocyte derived exosomes are packaged with about 40 μM each of gRNA RNPs.

In some aspects, administering can be intravenous administration. In some aspects, administering can be intramuscular, intra-liver, subcutaneous, intraperitoneal, oral, or nasal. Thus, in some aspects, the therapeutically effective amount of cell (e.g. hepatocyte) derived exosomes is present in a composition, such as a pharmaceutically acceptable composition, formulated for the specific delivery type as described herein.

In some aspects, any of the exosomes or compositions disclosed herein can be used in the disclosed methods.

### G. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. Disclosed are kits comprising recombinant exosomes comprising a NTCP binding motif. Disclosed are kits comprising hepatocyte-derived exosomes comprising a NTCP binding motif.

Disclosed are kits comprising hepatocyte-derived exosomes comprising an NTCP binding motif fused to a transmembrane protein wherein the transmembrane protein is CD9, CD63 or CD81. Disclosed are kits comprising hepatocyte-derived exosomes comprising a NTCP binding motif fused to a transmembrane protein wherein the transmembrane protein is a truncated CD9.

Disclosed are kits comprising exosomes comprising a NTCP binding motif, wherein the NTCP binding motif is fused to a transmembrane protein. In some aspects, the presence of a transmembrane protein allows for the NTCP binding motif to be positioned, or anchored, on the outside of the exosome.

Disclosed are kits comprising exosomes comprising a NTCP binding motif as described herein and further comprising a gRNA/Cas ribonucleoprotein complex comprising a gRNA and a Cas protein

In some aspects, the kits can comprise one or more of the exosomes, nucleic acids, or compositions described herein. In some aspects, the kits can comprise hepatocytes or other cell types suitable for producing the disclosed exosomes.

### EXAMPLES

Patients with HBV infection are subject to prolonged (often lifelong) treatment due to the lack of curative therapeutics. Thus, multimodal, robust therapeutic strategies are urgently needed to eliminate HBV cccDNA, which sustains viral replication and is refractory to the current antiviral regimens. Recently, a specific and potent gRNA/Cas9 HBV gene-editing drugs using HBV cellular models has been designed. An exosome-based drug delivery system that can specifically deliver these synthetic gRNA/Cas9 RNP drugs to human hepatocytes has also been developed. The antiviral efficacy and any potential off-target effects of these dual specific HBV gene-editing drugs can be evaluated, both in vitro and in vivo. The objective of this project is to lay the foundation for future clinical studies to achieve the long-term goal of curing chronic HBV infection. If successful, this novel drug design and delivery platform may be utilized to combat existing and emerging viral diseases. It is thus clinically significant, timely, and relevant to public health.

### A. Example 1: Development of Engineered Exosomes Carrying Synthetic gRNA/Cas9 Drugs

### 1. Background

The objective of this study is to develop engineered exosomes carrying synthetic gRNA/Cas9 drugs capable of abolishing hepatitis B virus (HBV) replication in infected hepatocytes. HBV covalently closed circular (ccc) DNA sustains HBV replication in human hepatocytes and is refractory to the current antiviral nucleos(t)ide analog (NA) regimens. Thus, new curative strategies must focus on eliminating cccDNA from HBV-infected hepatocytes. CRISPR/Cas9 technology is an appealing approach for targeted gene-editing, but its application in humans is limited due to non-specific drug delivery and off-target gene-editing (side) effects. The current CRISPR/Cas9 delivery approaches often require viral vectors, which pose safety concerns for therapeutic applications in humans. Also, the viral- and/or plasmid-based, long-term CRISPR/Cas9 expression systems can increase off-target effects and elicit immune responses. Notably, synthetic gRNA/Cas9 ribonucleoprotein (RNP) is a non-viral formula with excellent features, including rapid DNA cleavage, low off-target effects, low risk of insertional mutagenesis, easy production, and readiness for clinical use. A series of gRNA/Cas9 HBV gene-editing RNPs has been designed and tested, and the most specific and potent gRNA/Cas9 RNP drugs for reducing HBV cccDNA and transcripts in HBV-infected cells have been selected. However, the existing viral and non-viral delivery systems for gRNA/Cas9 RNPs have several shortcomings-such as non-specific (off-site) delivery, limited drug-loading capacity, low biocompatibility, poor stability, cytotoxicity, and potential for immunogenicity. These shortcomings limit the use of synthetic gRNA/Cas9 RNPs for in vivo applications. To overcome these obstacles, a novel exosome-based system has been developed to deliver synthetic gRNA/Cas9 RNPs specifically to HBV target cells (i.e., human hepatocytes). The capability of these exosome-based HBV gene-editing gRNA/Cas9 RNPs (herein called Exo-HBV-Eliminator) can be evaluated to target human hepatocytes, their biophysical and biological properties can be defined, and their antiviral efficacy and potential cytotoxicity can be assessed, using both cellular and animal models.

### 2. Premise:

Curing chronic HBV infection is a major challenge and unmet medical need due to the lack of effective and specific therapeutics that can eliminate HBV cccDNA from infected hepatocytes. In this project, a series of gRNA/Cas9 RNPs that can specifically target and efficiently excise HBV cccDNA were designed, their antiviral activities were tested in HBV cellular models, and the most specific and potent candidates were selected. Also, a novel exosome-based delivery system engineered to carry HBV pre-S1-derived peptides (PSIP) on the surface of exosomes so that they can specifically target HBV receptor (sodium taurocholate co-transporting polypeptide/NTCP) expressed on human hepatocytes was designed. Importantly, these engineered exosomes exhibited much higher specificity in targeting HepG2-NTCP cells (compared to HepG2 cells without NTCP) than exosomes without PSIP. Moreover, the engineered exosomes carrying selected gRNA/Cas9 RNPs efficiently reduced HBV cccDNA biogenesis and HBV replication in HBV-infected HepG2-NTCP cells. The antiviral efficacy and cytotoxicity of the exosome-based gRNA/Cas9 drugs can be further evaluated using HBV cellular and animal models.

The Exo-HBV-Eliminator can specifically target and efficiently abolish viral replication in HBV-infected hepatocytes and elicit minimal cytotoxic effects both in vitro and in vivo. This can be shown with biophysical characterizations and evaluation of the Antiviral and Off-Target Effects.

Determination of the Biophysical and Biological Properties of the Exo-HBV Eliminator: Biophysical characterizations can include the exosome's drug loading capacity, human hepatocyte targeting, cellular uptake, and drug release kinetics. The antiviral efficacy (HBV cccDNA insertion/deletion) and potential cytotoxicity of these exosome-based RNP drugs can be assessed using multiple HBV-infected cellular models.

Evaluation of the Antiviral and Off-Target Effects of the Exo-HBV Eliminator in HBV-Infected, Liver-Humanized Mice In Vivo: The Exo-HBV-Eliminator can be administered intravenously into HBV-infected, liver-humanized NRG-Fah KO (Fah/NRG-hu HEP) mice. Next, the tissue/cell biodistribution and half-life of the candidate gRNA/Cas9 RNPs can be kinetically determined, including in vivo trafficking and accumulation/retention in humanized liver/hepatocytes, and most importantly, their antiviral efficacy and potential side effects in treated animals.

Patients with chronic HBV infection have to endure a prolonged (often lifelong) treatment due to the lack of therapeutics that can target HBV cccDNA. This study is innovative and can have a high impact on HBV treatment by addressing the current bottlenecks in HBV gene therapy: i.e., off-target drug delivery and no effect on HBV cccDNA. It is anticipated that the engineered exosomes carrying synthetic gRNA/Cas9 RNPs can have improved hepatocyte targeting and uptake/retention capabilities, exert specific on-target editing of HBV cccDNA, and elicit very low off-target side effects on human hepatocytes. The overarching goal is to develop an exosome-based HBV gene-editing drug capable of abolishing HBV cccDNA replication to functionally cure HBV infection.

### B. Example 2: Development of an Exosome-Based HBV Gene-Editing gRNA/Cas9 RNP Drug

### 1. Significance

i. The Dilemma of Curing HBV Infection with the Current Antiviral Nucleos(t)ide Analog (NA) Regimens

Hepatitis B virus (HBV) chronically infects nearly 300 million people worldwide, resulting in one million deaths annually, owing to liver cirrhosis and hepatocellular carcinoma (HCC). The current antiviral treatment (using NA regimens) can only suppress de novo HBV replication but cannot eliminate HBV infection due to the persistence of HBV covalently closed circular (ccc) DNA, which sustains HBV replication. Distinct from integrated HBV DNA that may encode only subgenomic transcripts (HBsAg), the cccDNA (an episomal viral minichromosome) serves as the genuine template for transcribing all HBV mRNAs, including the pre-genomic RNA (pgRNA) that serves as a reverse transcription template. Thus, HBV cccDNA is the key therapeutic target for eradicating HBV infection. Thus far, the molecular mechanisms of HBV cccDNA biogenesis in infected cells remain unclear, and there are no available drugs that can directly target HBV cccDNA. Therefore, curative strategies focused on eliminating HBV cccDNA are urgently needed to treat HBV-infected cells without inducing cytotoxic effects.

ii. The Challenges Facing the Application of CRISPR/Cas9-Mediated Gene-Editing to Combat HBV Infection

Amongst the genetic approaches (e.g., Zn finger/TALENs/homing endonucleases, recombinases, CRISPR/Cas9) being employed for targeted gene-editing, CRISPR/Cas9 is an appealing approach to combat HBV infection. However, this approach faces several challenges that need to be overcome before its clinical application, in particular drug delivery specificity and potential off-target effects. Current CRISPR/Cas9 expression/delivery systems often require viral vectors, which can lead to off-target effects, cytotoxicity, long-term expression, immunological responses, and insertion/deletion (indel) mutagenesis or oncogenesis, raising safety concerns for application in humans. Notably, synthetic gRNA/Cas9 ribonucleoprotein (RNP) is an appealing non-viral formulation with multiple advantages, including rapid DNA cleavage, decreased off-target effects, low risk of indel mutagenesis, easy synthesis and gRNA multiplexing, and readiness for clinical use. Being exciting options, existing non-viral vectors that can deliver these gRNA/Cas9 RNPs (e.g., liposomes or nanoparticles) face several challenges, such as off-site delivery, limited cargo-loading capacity, poor biocompatibility/stability, cytotoxicity, and potential immunogenicity. These challenges limit the in vivo use of synthetic gRNA/Cas9 RNPs for clinical applications. Thus, designing specific gRNA/Cas9 RNP modalities and developing a vehicle for their delivery are urgently needed for eliminating HBV infection from reservoir cells and eradicating chronic HBV infection.

iii. Engineered Exosomes are Excellent Vehicles for the Delivery of gRNA/Cas9 RNPs to Target Cells

Exosomes are a subtype of nanoscale membranous vesicles naturally released from the endocytic compartment of live cells, and their cargos (DNA, RNA, proteins, and lipids) are reflective of their cell-of-origin. Exosomes are considered promising delivery vehicles (especially for the large size of RNPs) because they circumvent most of the limitations associated with currently available viral and non-viral vectors. For example, immune responses to Cas9 RNPs and delivery vectors are a major concern in CRISPR/Cas9-mediated gene therapy, as they can neutralize the effectiveness of the drugs or potentially cause serious side effects. Unlike viral vectors, exosome-mediated delivery of synthetic gRNA/Cas9 RNPs robustly overcomes immunogenicity concerns as it provides an immune-privileged protection. Unlike synthetic nanoparticles, exosomes are immunologically inert and non-cytotoxic, if purified from a compatible cell source. Unlike liposomes, exosomes carry various membrane-anchored proteins that extend their half-life in blood/circulation by evading phagocytic clearance while conferring superior cellular uptake and efficient delivery of their internal cargos to the recipient cells. Notably, exosomes can cross stringent biological barriers and can be engineered to deliver encapsulated gRNA/Cas9 RNPs specifically to target cells. Previous studies have demonstrated the ability of engineered exosomes to deliver therapeutics for targeted cancer therapy. Thus, engineered exosomes may serve as an ideal vehicle for delivery of synthetic gRNA/Cas9 RNPs specifically to HBV target cells to achieve HBV cccDNA inactivation and excision/degradation. The keys for eliminating HBV infection by CRISPR/Cas9 technology are: A) selecting gRNAs targeting HBV genes that are crucial for viral replication but do not overlap with the human genome (to avoid off-target effects); and B) delivering these gene-editing drugs specifically to HBV reservoir cells. To achieve this, a series of gRNA/Cas9 RNPs was designed and selected for the most specific/potent candidates that can reduce HBV cccDNA in HBV-infected hepatocytes; 2) a novel exosome-based delivery system engineered to specifically target human hepatocytes was developed. These exosomes are engineered in such a way that an HBV pre-S1-derived peptide (PS1P) is expressed on their surface to recognize and bind HBV receptor NTCP (Na+taurocholate co-transporting polypeptide) on human hepatocytes, so they can specifically deliver and intracellularly release synthetic gRNA/Cas9 RNPs into hepatocytes and 3) HBV cellular and animal models and HBV on/off-target detection methods suitable for testing the efficacy and cytotoxicity of these HBV gene-editing gRNA/Cas9 RNPs (herein called Exo-HBV-Eliminator) were developed. The biophysical and biological properties of the Exo-HBV-Eliminator in HBV cellular models can be evaluated. Next, the antiviral and off-target (side) effects of the Exo-HBV-Eliminator in HBV-infected, liver-humanized mice can be evaluated.

### 2. Innovation

i. Use of Synthetic gRNA/Cas9 RNPs to Target HBV cccDNA and/or iDNA

Conceptually, synthetic gRNA/Cas9 RNPs do not rely on the cellular transcriptional/translational machinery for HBV gene-editing. This novel approach can ensure that the synthetic gRNA/Cas9 RNPs do not carry any long nucleic acids or virus-derived DNA sequences that could integrate into the host cell genome. Also, synthetic gRNA/Cas9 RNPs represent a transient form of gene-editing drug that can be administered in a controllable way to avoid the risk caused by a sustained expression of Cas9 nuclease, which might generate unwanted off-target effects and/or immune responses. Practically, designing and selecting specific gRNAs is key to a successful HBV gene-editing. Using a direct transfection approach, a series of gRNA/Cas9 RNPs have been designed and screened and the most specific and potent candidates that can dramatically reduce HBV cccDNA and/or iDNA identified in infected hepatocytes. Because delivery of RNP therapeutics is currently bottlenecked by the large size of the RNPs (exceeding the loading capacity of both viral and non-viral vectors), using exosome-mediated delivery in established HBV cell and animal models creates a high translational potential for their clinical application.

ii. Use of an Engineered Exosome Drug Delivery System to Target HBV Reservoir Cells

Efficient delivery of CRISPR/Cas9 RNPs to target cells is a major obstacle in unlocking its translational potential for HBV gene therapy. An ideal vehicle must be safe and highly efficient in delivering the synthetic gRNA/Cas9 RNPs to HBV reservoir cells, offering on-target specificity and minimizing off-target effects. An engineered exosome system for specific delivery of synthetic gRNA/Cas9 RNPs to HBV target cells has been developed. These exosomes carry an HBV-PSIP, which binds to the HBV receptor (NTCP) expressed on human hepatocytes as well as on other types of cells that may express HBV receptor. This novel approach can confer HBV tropism-dependent drug delivery to HBV target cells in HBV-infected hosts.

### 3. Approach

i. Selecting gRNAs Targeting HBV DNA without Potential Off-Targeting the Human Genome

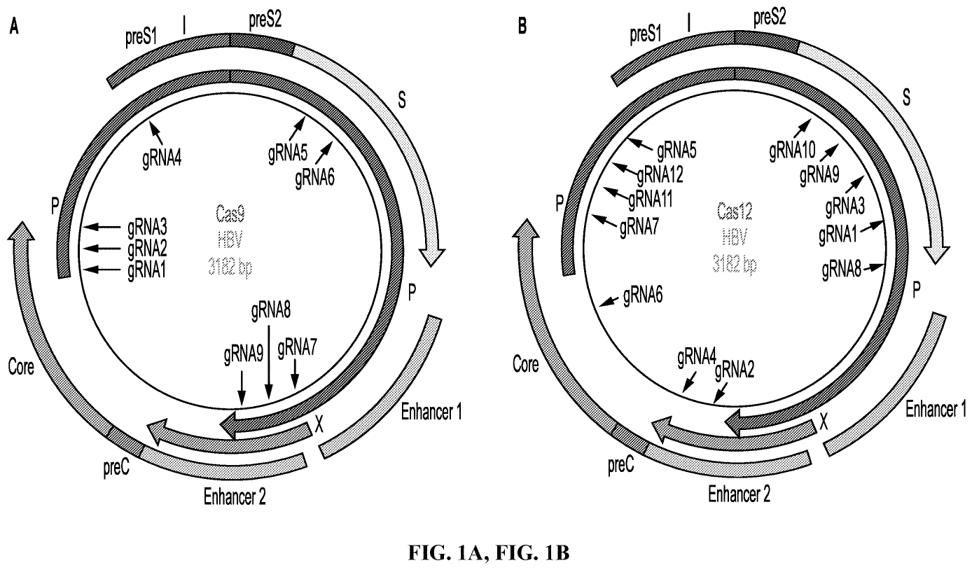

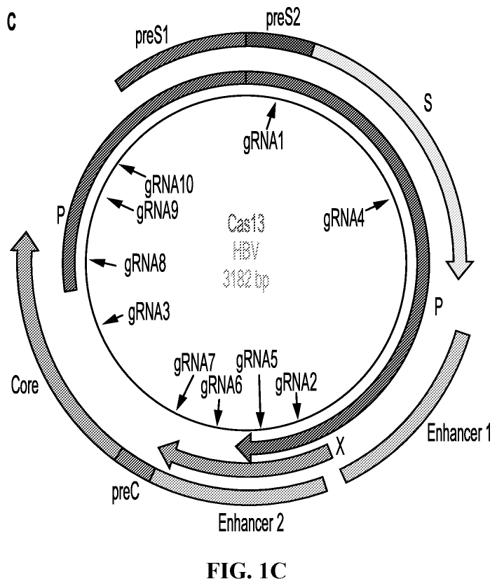

Identifying specific gRNAs for Cas9 nuclease to cleave HBV DNA without causing off-target effects is the first step in designing CRISPR/Cas9-based therapeutics for HBV eradication. The heterogeneity of the HBV genome (i.e., HBV quasispecies) complicates the design of gRNAs that can target multiple or all HBV genotypes. To select the most specific targets in the HBV genome, an e-CRISPR program (CHOPCHOP) was used to analyze HBV sequences that have the best-predicted on-target (i.e., HBV sequences that are critical for viral replication) and lowest off-target effects (i.e., 0% homology within the human genome). The genomic DNA sequences of 8 HBV genotypes (A-H) originating from distinct geographic regions worldwide were compared, and 9 target sites in highly conserved regions of the HBV genome within the polymerase (P), capsid (C), surface(S), and X genes (FIG. 1A) were identified. 9 gRNAs (based on the ayw strain, Genbank accession number: NC_003977.2) that are specifically designed with a protospacer adjacent motif (PAM) were synthesized, which can be recognized by the Cas9 nuclear localization signal (NLS) that can direct the gRNA/Cas9 RNP into the cell nucleus. The antiviral activities and off-target effects of these gRNAs were tested, two gRNAs that have the most specific and potent antiviral effects in HBV cellular models were identified. gRNAs for Cas12 (FIG. 1B) that edits DNA and Cas13 (FIG. 1C) that edits RNA can also be selected. A combination of these selected gene-editing drugs can have synergy effect on antiviral activity because multiple sites of viral genomes are cut and difficult to repair by the host DNA and RNA repair mechanisms.

ii. Synthetic gRNA/Cas9 RNPs Abolish HBV Replication in HepDE19 Cells

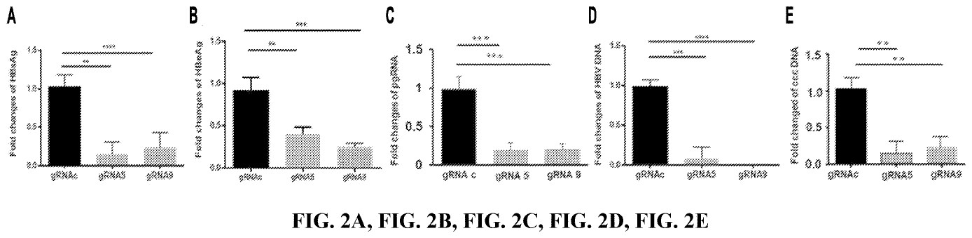

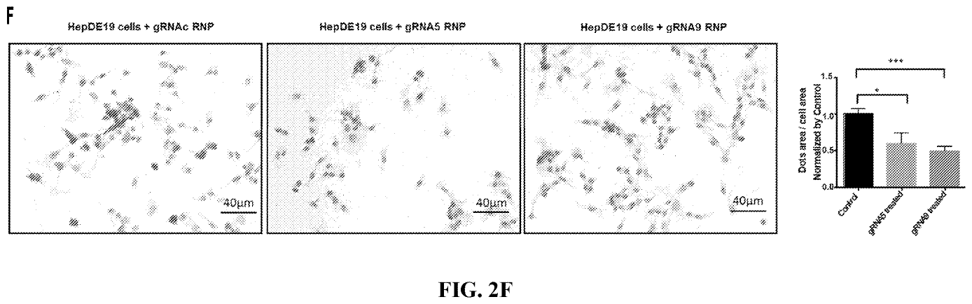

Among the 9 gRNAs tested in the studies, gRNA5 (targeting HBV P/S genes) and gRNA9 (targeting HBV P/X/E2 genes) exhibited the most specific and potent antiviral efficacy in HepG2.2.15 cells, and thus these two gRNAs were selected for further testing in the HepDE19 cell model. As shown in FIG. 2, HBV replication was significantly suppressed in HepDE19 cells transfected with gRNA5/Cas9 or gRNA9/Cas9 compared with the scrambled gRNA control (gRNAc)/Cas9, as demonstrated by the significant decreases in the levels of HBsAg (FIG. 2A), HBeAg (FIG. 2B), pgRNA (FIG. 2C), total HBV DNA (FIG. 2D), cccDNA (FIG. 2E), and HBV mRNA (FIG. 2F) following incubation with Tet-free media for 3 days (to induce HBV replication). The designed gRNAs for Cas9, Cas12, and Cas13 showed 0% overlap with, and thus may have the lowest off-target effects on, the human genome. GFP-labelled Cas9 (Spy GFPPRO Cas9), Cas12a (EnGen Lba Cas12a), and Cas13d (from Ruminococcus flavefaciens XPD3002) proteins are commercially available from Millipore-Sigma, New England BioLab, and MCLAB, respectively. The on-target antiviral activity and off-target cytotoxic effects of these HBV-specific gRNA/Cas systems can be tested and compared with scrambled gRNA control (gRNAc/Cas) in vitro using HBV-infected cell models and in vivo using a humanized animal model.

iii. HepDE19 Cells Treated with Synthetic gRNA/Cas9 Produce Fewer Copies of Infectious HBV Particles

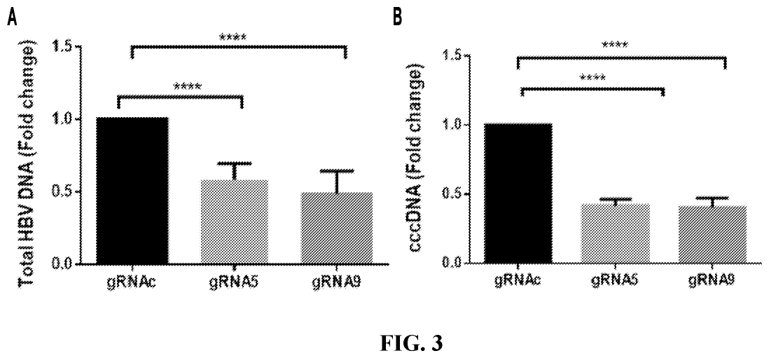

To characterize the effects of synthetic gRNA5/Cas9 and gRNA9/Cas9 on HBV replication, the ability of HBV particles derived from HepDE19 cells that have been transfected with gRNA5, gRNA9, or gRNAc RNPs for 3 days to infect HepG2-NTCP cells was assessed. HepG2-NTCP cells were infected for 6 days with supernatants harvested from HepDE19 cells. Genomic DNA was isolated from HepG2-NTCP cells and used for qPCR quantification of HBV DNA. It was found that HepG2-NTCP cells treated with supernatants of gRNA5/Cas9- or gRNA9/Cas9-transfected HepDE19 cells had significantly fewer copies of HBV DNA (FIG. 3A) and HBV cccDNA (FIG. 3B) than cells treated with supernatants from the gRNAc-transfected cells. These data show that gRNA5 and gRNA9-treated HepDE19 cells produce fewer copies of infectious HBV particles capable of infecting HepG2-NTCP cells.

iv. Synthetic gRNA/Cas9 RNPs Delivered to HBV-Infected HepG2-NTCP Cells by Direct Transfection or by Exosomes Inhibit HBV cccDNA

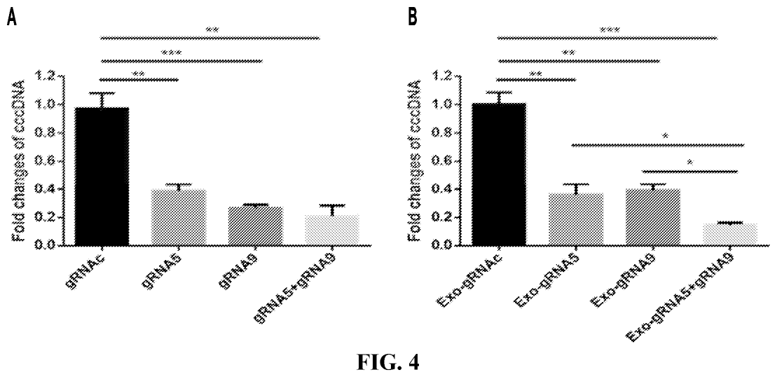

To test whether the synthetic gRNA/Cas9 can reduce newly formed HBV cccDNA in HepG2-NTCP cells, the cells with HBV particle-containing supernatants from HepDE19 cells (supernatants harvested 6 days after culture in Tet-free media) were infected. The HepG2-NTCP cells were infected with HBV particles for 6 days and then transfected with gRNA5, gRNA9, gRNA5 plus gRNA9 (in combination), or gRNAc RNPs for 3 days, followed by measuring HBV cccDNA. In another experiment, the infected HepG2-NTCP cells were incubated for 3 days with exosomes packaged with gRNA5, gRNA9, gRNA5 plus gRNA9 (in combination), or gRNAc RNPs, followed by measuring HBV cccDNA. The introduction of gRNA5, gRNA9, or gRNA5 plus gRNA9 RNPs into HepG2-NTCP cells, either by direct transfection (FIG. 4A) or via delivery by engineered exosomes (FIG. 4B), significantly reduced HBV cccDNA biosynthesis. These data indicate that gRNA/Cas9 delivery using the engineered exosomes elicits similar or even better antiviral efficacy (with gRNA5 plus gRNA9 showing the best antiviral effect) compared with the direct transfection approach.

v. Determining Insertion/Deletion (Indel) Mutations and Gene-Editing in HBV DNA by Synthetic gRNA/Cas9 RNPs

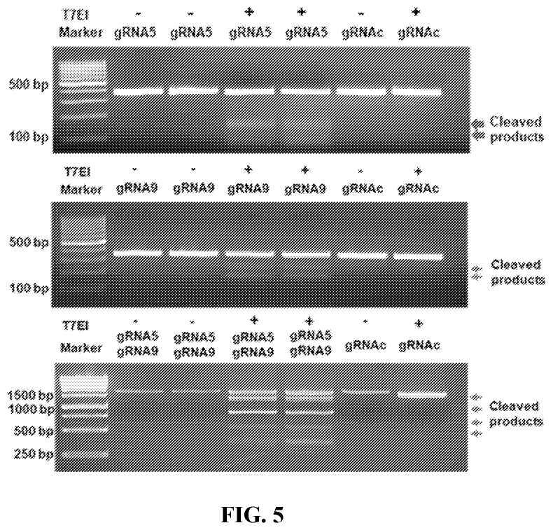

DNA cleavage by CRISPR/Cas9 results in double-strand breaks (DSBs), which are repaired primarily by the non-homologous end-joining (NHEJ) pathway, resulting in indel mutations and transcriptionally active episomal variants. Thus, detecting DNA cleavage and indel mutations following CRISPR/Cas9-mediated DNA damage and repair is important for confirming its gene-editing effects. To validate the capability of the synthetic gRNA/Cas9 RNPs to introduce indel mutation and cleavage in HBV DNA in HepDE19 cells, a mismatch sensitive T7 endonuclease (T7E1) assay-was performed, following transfection of HepDE19 cells with gRNA5, gRNA9, gRNA5+gRNA9, or gRNAc RNPs. This assay allows for the detection of specific cleavage sites or indel mutations in the target gene (HBV DNA) in the treated cells. The indel mutagenesis was determined based on the DNA fragments detected after the gRNA/Cas9-mediated cleavage of the HBV DNA following T7E1 digestion of the specific PCR products. As shown in FIG. 5, cleaved products were observed in HBV DNA by T7E1 treatment following transfection with gRNA5, gRNA9 or gRNA5+gRNA9 but not in the gRNAc-transfected cells or cells without T7E1 treatment, indicating specific cleavage and/or indel mutations in HBV DNA. Sanger sequencing of these HBV DNA fragments revealed specific DNA indel mutations in the gRNA5 or gRNA9, or gRNA5 plus gRNA9 RNP-targeted sites, confirming unrepaired DNA damages and targeted DNA cleavage. These results demonstrate that the reductions detected in the HBV products (DNA/RNA/antigens) are due to specific HBV gene-editing by the gRNA/Cas9 RNPs.

vi. Synthetic gRNA/Cas9 RNPs do not Exert Detectable Cytotoxic Effects in HepDE19 Cells

To determine whether the synthetic gRNA/Cas9 RNPs exert any significant cytotoxic effects on HepDE19 cells, cell viability was measured after treatment with gRNA5/Cas9 or gRNA9/Cas9 RNPs using the MTT assay. Compared with the non-transfected (NT) control cells, no differences in the viability of HepDE19 cells transfected with gRNA5/Cas9, gRNA9/Cas9, or gRNAc, and cultured with or without Tet-free (for HBV induction) for 24 hours were observed. Since HBV itself is not directly a cytopathic pathogen but induces cell injury via immune-mediated mechanisms, cell viability of the transfected and non-transfected HepDE19 cells that were cultured in Tet-free medium in the presence of TNFα (to induce apoptotic cell death) was assayed. No cytotoxic effects from gRNA5, gRNA9, or gRNAc control transfection were observed. Moreover, flow cytometry analysis was performed to evaluate cell apoptosis by measuring Av and 7ADD levels in transfected HepDE19 cells with or without Tet-free HBV induction. No significant differences in cell apoptosis among the different treatments were observed. Finally, the proliferation of HepDE19 cells following transfection with or without gRNA/Cas9 for 3 days was assayed, using the MTT assay. No significant changes in cell proliferation were found. Taken together, these results indicate that the synthetic gRNA5/Cas9 and gRNA9/Cas9 RNPs are not cytotoxic to hepatocytes.

vii. Engineering Exosomes to Deliver Synthetic gRNA/Cas9 RNPs Targeting NTCP⁺ Hepatocytes

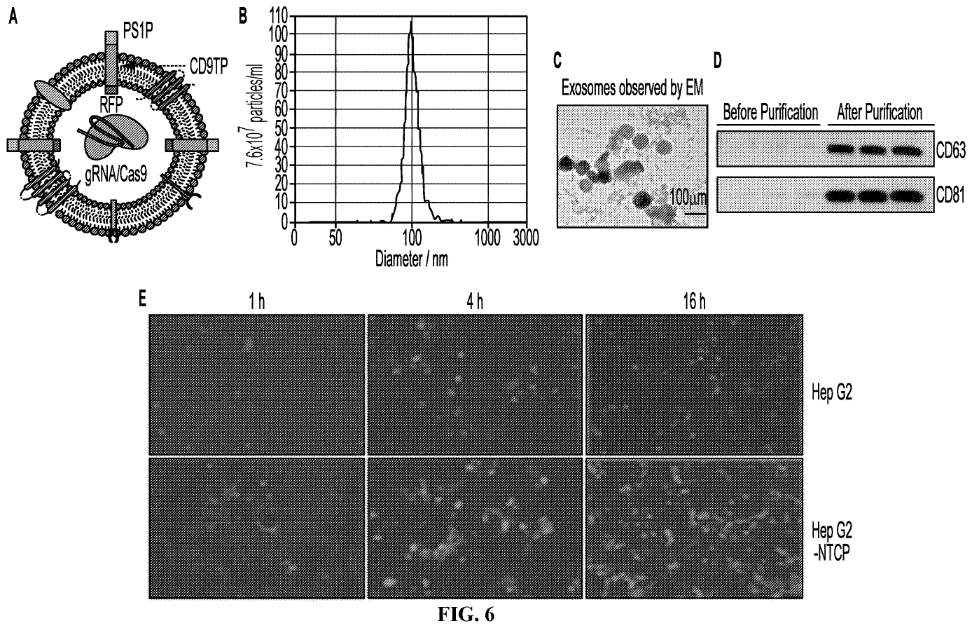

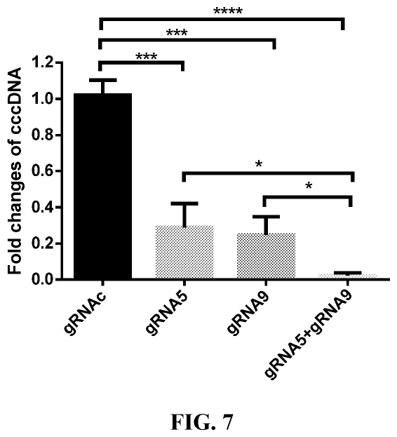

To deliver the synthetic gRNA/Cas9 RNPs to human hepatocytes, a novel exosome-based delivery system that functions as a natural cell-derived nanocarrier was developed. To this end, a plasmid expressing HBsAg pre-S1-peptide (PSIP) that can specifically bind to the HBV receptor (NTCP) expressed on human hepatocytes was first designed. Specifically, a plasmid expressing a fusion protein comprising three components was constructed: an extracellular PS1P, a CD9 transmembrane protein (CD9TP), and an intracellular red fluorescent protein (RFP) (FIG. 6A). The plasmid was constructed in such a way that the DNA sequence encoding HBV PS1P was synthesized (IDT DNA Technologies) with specific restriction enzyme (Xba1 and EcoR1) recognition sites at both ends for subcloning into the N-terminal of CD9TP vector. The N-terminus (amino acids 1-37) in CD9TP was truncated to expose the PSIP on the exosome surface because these amino acids are embedded in the exosomal membrane. Because exosomes purified from a compatible cell source elicit minimal cytotoxicity and immunogenicity and hepatic-derived cells exhibit inherent liver biocompatibility and a high yield of liver homologous exosomes, the constructed plasmid was transfected into primary human hepatocytes (PHHs), which were cultured using the FiberCell System that can produce large quantities of exosomes capable of targeting human hepatocytes specifically. The exosomes were isolated from the cell culture supernatants using differential ultracentrifugation (Beckman) and EV column purification (Invitrogen). The size (~100 nm) and concentration (7.6×10⁷ particles/ml) of the purified exosomes were measured by the nanoparticle tracking analysis (NTA) (FIG. 6B). The shape and size of the purified exosomes were confirmed by Tecnai F20 electron microscope (EM) observation (FIG. 6C). Immunoblotting revealed that the exosome markers CD63 and CD81 were highly concentrated in the purified exosomes (FIG. 6D). Next, gRNA5/Cas9 and gRNA9/Cas9 (individually or in combination) were electroporated into the purified exosomes according to a published method. Upon adding to cell cultures, these engineered exosomes specifically targeted (retained/accumulated) in the HepG2-NTCP cells, but minimally presented (or held) in the HepG2 cells without NTCP (FIG. 7E). Importantly, the exosomes packaged with gRNA/Cas9 RNP drugs significantly reduced the levels of HBV cccDNA in HBV-infected HepG2-NTCP cells (FIG. 4B).

### 4. Research Strategy

i. Overall Design:

Targeting specificity and drug delivery of CRISPR/Cas9 RNPs is critical for its clinical application as a gene-editing drug for eradicating HBV infection. The goal of this is to develop an exosome-based HBV gene-editing gRNA/Cas9 RNP drug (Exo-HBV-Eliminator) that can specifically target and cleave HBV DNA in infected hepatocytes. In the preliminary studies, exosomes as a drug delivery vehicle were engineered and found to specifically deliver the synthesized gRNA/Cas9 RNPs to hepatocytes expressing the HBV receptor NTCP (FIG. 6E), because these exosomes carry an HBV PSIP that has a high NTCP-binding affinity. Also, the preliminary studies demonstrated that this Exo-HBV-Eliminator formulation efficiently reduced HBV cccDNA levels (FIG. 4B). Based on these preliminary studies, it is hypothesized that this engineered Exo-HBV-Eliminator can specifically target and efficiently abolish viral replication in HBV-infected cells without apparent cytotoxic effects, both in vitro and in vivo. The exosome-based HBV gene-editing drugs can be used to eliminate HBV (ccc) DNA from HBV-infected hepatocytes, laying the foundation for achieving an HBV cure.

ii. Scientific Rigor, Reproducibility, and Statistics

The experiments can be rigorously designed to yield robust and unbiased results, including appropriate negative controls (scrambled gRNAc/Cas) and uninfected cells). All experiments can incorporate the use of independent technical and biological replicates. The experiments can be powered to see expected differences, and the prospective sample size estimation can be performed in consultation with a Ph.D.-level biostatistician. Sample size can be increased if the statistical power cannot be warranted. A paired t-test or student t-test to can be used to compare the measures in two groups of cells/animals with or without experimental treatment. Differences among multiple groups can be determined by ANOVA following appropriate multiple comparisons. Relevant biological variables, such as age- and sex-based differences can be considered within the study groups.

iii. Evaluation of the Biophysical and Biological Properties of the Engineered Exo-HBV Eliminator In Vitro

The most specific/potent gRNA/Cas9 RNPs that can significantly reduce HBV cccDNA have been designed and selected. A new exosome-based drug delivery system that can deliver the drug specifically into hepatocytes (expressing HBV receptor NTCP), for in vivo application has also been developed. To date, the biophysical characteristics of these engineered exosomes, such as the exosome drug loading capacity, PHH targeting, cellular uptake, and drug release kinetics, remain to be determined pre-clinically. It was found that one-time transfection with a single dose of gRNA5 or gRNA9 RNPs in HBV-infected hepatocytes resulted in >60% reduction in HBV transcripts (DNA/RNA/antigens) compared with one-time transfection with gRNAc (FIG. 2-4). Repeated treatments with a combination of gRNA5+gRNA9 RNPs (i.e., dual DNA excision) resulted in a more pronounced reduction (>95%) of HBV cccDNA, but this synergy effect was not observed in HepDE19 cells following repeated treatments with gRNA5 or gRNA9 RNP alone (FIG. 7). Sanger DNA sequencing confirmed specific HBV gene-editing/excision at the targeted sites of both gRNAs. Therefore, it is likely that gRNA/Cas9-mediated HBV gene-editing by a single gRNA treatment generates new HBV variants that are transcriptionally capable but less competent in supporting HBV cccDNA biosynthesis and replication. To validate these results and improve HBV eradication, these studies can be expanded to optimize the antiviral protocol using exosomes packaged with dual gRNA5+gRNA9 RNPs and administer multiple doses in HBV-infected PHHs (the HBV target cells). Additionally, since off-target effects are always a concern for any genetic-based approach, evaluation of any potential off-target effects and cytotoxicity of the Exo-HBV-Eliminator in treated PHHs is essential. The result can be a gene-editing drug that can be used clinically for HBV curative therapy.

iv. Biophysical Characterization of the Exo-HBV-Eliminator

Specific delivery and sustained release of the gRNA/Cas9 RNPs to HBV-infected hepatocytes are critical factors in developing an effective CRISPR-based HBV gene therapy. One aspect of this translational study is based on using PHHs (for better production rate and liver compatible) to produce exosomes packaged with synthetic gRNA/Cas9 RNPs (that can specifically target and efficiently cleave HBV cccDNA) and PSIP (an HBV preS1 peptide that has a high affinity for HBV receptor/NTCP). Therefore, these exosomes possess high targetability toward human hepatocytes expressing HBV receptor NTCP. Thus, the biophysical features of these PHH-derived exosomes can be characterized as well as their capability in encapsulating synthetic gRNA/Cas9 RNPs, testing their capacities to deliver and release the gRNA/Cas9 RNP cargos into PHHs. HepG2 or Huh7 hepatoma cells without NTCP receptor can serve as a negative control.

For morphological characterization, exosomes purified from PSIP-CD9TP-RFP-transfected PHHs can be processed and visualized by Zeiss-EM10C transmission electron microscopy (TEM) and scanning electron microscopy (SEM). The exosome size and concentration (particles/ml) as well as stability by Zeta View NTA (Malvern NanoSight) and dynamic light scattering (DLS) can then be determined. This NTA system was recently used to observe the size and concentration of the purified exosomes and electron microscopy was used to observe the exosome shape and size. To define the biophysical features, immunophenotyping of the exosome proteins CD9, CD63, and CD81 (typical exosomal markers) can be performed. Flow cytometry, immunoblotting and immuno-electron microscopy (IEM) can be used to characterize exosomes purified from plasma of HCV patients. As such, the same approaches to characterize the PHH-derived exosomes can be used. To determine whether the gRNA/Cas9 RNPs are efficiently loaded onto the exosomes, the amount of Cas9 protein can be measured by immunoblotting of the loaded exosomes. The drug-loading capacity (DLC), drug-loading efficiency (DLE), and drug-loading stability (DLS) of these loaded exosomes can be measured using previously published methods. In addition to functionally assaying the uptake/retention of PS1P⁺ exosomes in NTCP⁺ cells (FIG. 6E), custom-made, gold- or bead-labeled anti-HBV preS1 mAb (Creative BioLabs) can be used to perform IEM and imaging flow cytometry (using the Amnis imager) to characterize the presence of PSIP on the membrane of the exosomes. The kinetics of cellular uptake of the RFP-labelled Exo-HBV-gRNA/Cas9 drug can be assessed by confocal microscopy and imaging flow cytometry (AMNES). The drug release kinetics and half-life of the gRNA/Cas9 RNPs can be determined using gRNA/Cas9-Luc RNP (Millipore Sigma), and the luciferase activity in the treated hepatocytes can be determined by luciferase assay at different time points (1, 3, 6, 12, 24, 48, and 72 h) after the treatment. The amount of free gRNA/Cas9 RNP released (at different time points) from the exosomes after their incubation with the hepatocytes can be measured by liquid chromatography-tandem mass spectrometry and luciferase assay—to determine the abundance and stability of the exosomes and the gRNA/Cas9-Luc in the culture supernatant and cell lysate using published methods.

v. Biological Characterization of the Exo-HBV-Eliminator

The Exo-HBV-Eliminator drug can specifically target hepatocytes (FIG. 6) and reduce HBV cccDNA in HepG2-NTCP cells (FIG. 4B). Their effects on HBV-infected PHHs need to be evaluated to translate this new therapeutic into animal studies and clinical trials for achieving a complete HBV cure. The on-target analysis can be expanded by evaluating the antiviral efficacy in HBV-infected PHHs incubated with a combination of the Exo-HBV-Eliminator drug (1×10⁸ particles packaged with 40 μM each of gRNA5+gRNA9 RNPs), using either one-time or multiple-time treatments. The deliverability rate of the exosomes carrying PSIP versus control exosomes (without PSIP) can be compared. The antiviral activities and deliverability rate of the drugs in HepG2.2.15 and HepDE19 cells (both do not express NTCP) can also be determined. Traditional transfection and delivery methods (including jetPEI reagents and protocol) can be used to assess any effects from free (no exosomes) HBV-gRNA/Cas9 drug, as an additional control. In addition to measuring HBsAg/HBeAg expression by ELISA and HBV DNA/RNA by real-time PCR, the antiviral effect on HBV cccDNA can be evaluated (FIG. 2-4). Briefly, the protein-free HBV DNA can be extracted from whole cell lysates using the Hirt DNA extraction protocol and subjected to “gold standard” Southern blotting. The Hirt DNA prep can be treated either by plasmid-safe DNase and exonucleases (NEB) to remove the protein-free relaxed circular DNA, and the remaining cccDNA can be cleaned with a DNA clean and concentrator kit (ZYMO Research) and then subjected to qPCR amplification using primers and probe specific to HBV cccDNA. If the cccDNA copy number is below the qPCR detection limit, ultrasensitive digital droplet PCR (ddPCR) can be used with specific primers (forward: 5′-TCATCTGCCGGACCGTGTGC-3′ (SEQ ID NO: 49), reverse 5′-TCCCGATACAGAGCTGA GGCGG-3′ (SEQ ID NO: 50)), and internal probe (5′-FAM-TTCAAGCCTCCAAGCTGTGCCTTGGGTGGC-TAMRA-3′ (SEQ ID NO: 51)), which are designed for the detection of HBV cccDNA (genotype D, subtype ayw). The results can be analyzed by Bio-Rad's Quanta software (version 1.7.4), which allows the counting of the number of positive and negative droplets. Poisson statistics can be used to calculate the number of copies/μL based on a 20 μL 1×droplet digital PCR reaction. Serial dilutions of HBV plasmid can serve as an external quantification standard to accurately differentiate positive and negative clusters and eliminate false positive droplets. To investigate the direct impact of the Exo-HBV-Eliminator on cccDNA and assess the combinatorial effect of the drug (i.e., gRNA5+gRNA9) on HBV eradication, HBV-infected HepG2-NTCP or PHH cells (6 dpi) can be pretreated with/without tenofovir alafenamide (TAF) for 3 days to reduce the replicative DNA intermediates and enrich HBV cccDNA before the treatment with the Exo-HBV-Eliminator for 3 days (and the treatment can be repeated 3 times) to “clean up” the HBV replicative template in the infected hepatocytes. The amount of HBV RNA in the treated and untreated cells can be assessed by RNAscope, where QuPath bioimage software can be used to quantitively analyze HBV RNA signals from RNAscope images. This method has been used to assess HBV RNA levels in HepDE19 cells treated with gRNA/Cas9 RNPs (FIG. 2). ddPCR and RNAscope systems can measure viral DNA or RNA with greater sensitivity and specificity compared to the traditional Southern/Northern blot or in situ hybridization methods. These novel techniques, in conjunction with immunostaining and flow cytometry analysis of HBV antigens and cell phenotypic markers, can facilitate the detection and phenotyping of actively and inactively HBV-infected cells. To further validate the drug's antiviral capability, serially diluted supernatants from the Exo-HBV-gRNA/Cas9-treated, HBV-infected cells can be used to infect HepG2-NTCP or PHHs (FIG. 3) and then measure HBV cccDNA and transcripts (pgRNA and HBsAg/HBeAg) using the methods described above.

The efficiency and specificity of HBV gene-editing by the Exo-HBV-Eliminator at the designated sites within the HBV sequences can be verified by next-generation single-cell DNA sequencing. BD FACSAria Fusion cell sorter can be used to select the RFP-positive cells (containing Exo-HBV-Eliminator drug) and extract cccDNA for sequencing. The excision efficiency of the HBV cccDNA segments can be calculated as a ratio of the number of the sequence-verified fragments to all fragment numbers for each denoted experimental condition. With this definition, the excision efficiency can be considered as frequentist probability, i.e., the ratio of the frequency of occurrence of the event of interest to the total number of experimental repeats. This interpretation of excision efficiency can provide a predictive value, as it can be used to set a prior expectation on the success rate of each treatment for the excision of the desired segments of HBV cccDNA, and thus can indicate the likelihood of successful drug treatment. Hierarchical clustering can also be performed on the efficiency values of truncation events under different treatments and a scheme can be generated to group the efficiency values into a multilevel cluster tree represented by a dendrogram. A heat map can offer a predictive value for HBV elimination.

vi. On- and Off-Target Analysis in PHHs to Determine any Potential Cytotoxicity from Exo-HBV Eliminator

It has been previously reported that targeting HBV DNA by CRISPR/Cas9 results in double-strand breaks (DSBs), which are primarily repaired by NHEJ pathway and generate transcriptionally active episomal variants. Also, CRISPR/Cas9-mediated cleavage of HBV DNA may result in in-frame or out-of-frame mutations in the host cell genomic DNA. To investigate mismatch tolerance and off-target effects, a T7E1 mismatch cleavage assay can be performed to check for indel mutations (FIG. 5). Briefly, genomic DNA can be isolated from the treated cells following gene editing. The potential off-target regions can be PCR-amplified using specific primers and then analyzed by the T7E1 assay. The mutation rate can be calculated based on the grayscale intensity of the DNA band as follows: % gene modification=100×(1−(1−fraction cleaved)^{1/2}). PCR primers can also be designed and used for predicting potential off-target sites. In addition, the PCR products can be cloned into a TA vector or directly sequenced by SITE-seq or CIRCLE-seq and next-generation seq and analyzed by in silico bioinformatics tools.

To ensure a thorough assessment of any off-target effects, more than one hundred predicted sites that may be attributed to the DNA cleavage can be analyzed. The objective is to detect on-target as well as any potential off-target sites that might be induced by the Exo-HBV-Eliminator. This can be carried out by identifying genomic alterations, including structural variants (SVs), single nucleotide polymorphisms (SNPs), copy number variants (CNVs), and insertion/deletion (Indel) mutations under different treatments, and then comparing them to all potential off-targets. After thorough quality control steps, the resulting paired-end short-reads can be mapped to the human reference genome (Human_GIK-V37) by utilizing the Burrows-Wheeler Aligner (BWA) algorithm. The on-target to off-target ratio can then be calculated. At the cellular level, the drug cytotoxicity can be determined by the CellROX assay (for mitochondrial ROS production), MTT assay and proliferation assay (for cell viability), LDH release assay (for cell metabolism and survival), and Av/7AAD assay (for cell apoptosis).

vii. Evaluation of the Antiviral and Off-Target Effects of the Exo-HBV-Eliminator in HBV-Infected, Liver-Humanized Mice In Vivo

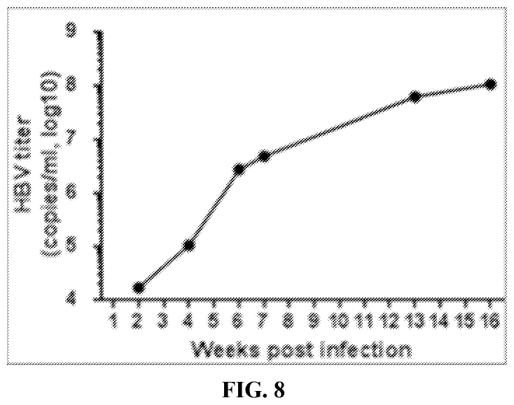

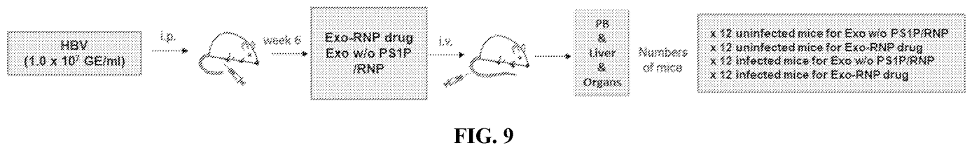

Using wild-type murine models for testing gene-editing drug delivery cannot be effectively translated to patients because these models lack human relevance. HBV fails to infect murine hepatocytes due to blockades at multiple steps of the HBV life cycle. Several chimeric liver-humanized mouse models have been developed. A liver-humanized mouse model has been established. This mouse model can be provided. These Fah/NRG-hu HEP mice were generated via engraftment/repopulation with human hepatocytes in NRG-KO (NOD.Rag1KO.IL2RγcKO)/Fah (Fumaryl acetoacetate hydrolase) KO mice (generated by using in vitro fertilization and CRISPR/Cas9 technology) that can be chronically infected with HBV (FIG. 8), indicating that this animal model readily supports HBV replication and thus can be used to test the HBV gene-editing drugs in vivo. Given that the engineered exosome-based drugs can specifically target and accumulate in HepG2-NTCP cells (FIG. 6) and significantly reduce HBV cccDNA in vitro (FIG. 4B), it is hypothesized that systemic administration of the PHH-derived Exo-HBV-Eliminator can effectively reach the liver and access the hepatocytes in HBV-infected, liver-humanized mice and eliminate/eradicate HBV infection. Therefore, the efficacy of the drug delivery in vivo can be evaluated by examining its tissue distribution and accumulation in hepatocytes of these liver-humanized mice. Additionally, the antiviral activity of this Exo-HBV-Eliminator following intravenous (i.v.) injection into HBV-infected, liver-humanized mice can be evaluated. Moreover, a tenofovir alafenamide (TAF) nano-drug ((NP-TAF, which inhibits HBV replication but not cccDNA can be used in combination with the Exo-HBV-Eliminator, i.e., applying a two-step/two-drug treatment approach. The antiviral efficacy, gene-editing activity, potential side effects, and immunogenicity of this drug combination can then be assessed.

viii. Determining Biodistribution and Safety of Exo-HBV-Eliminator in HBV-Infected, Liver-Humanized Mice

As an initial approach to determine the biodistribution of the Exo-HBV-Eliminator in vivo, the Fah/NRG-hu HEP mice can be infected with HBV via i.p. injection (107 genome equivalents (GE)/ml, genotype A, ayw strain), using de-identified HBV patient sera from Bioreclamation IVT (Hicksville, NY). After confirming positive HBV DNA/RNA/antigens in the sera (usually at week 2-4 after infection), at week 6 the Exo-HBV-Eliminator (0.9 nM RNPs in 100 μl vol) can be administered systematically via the tail vein. Mice receiving Exo-HBV-Eliminator containing RFP-Exo and luciferase-Cas RNPs can be visualized for RFP signal, and the luciferase luminescence signal can be kinetically quantified using an IVIS Spectrum In Vivo Imaging System (PerkinElmer), to determine the drug trafficking and accumulation in the humanized liver. The mice can be sacrificed 24 hours after administering the drug, and the blood and the tissues from major organs (including liver, kidney, spleen, and lymph nodes) can be harvested. Tissue cryosections or cell lysates can be prepared and examined by confocal microscopy to monitor the RFP-labeled Exo-HBV-Eliminator distribution or by luciferase assay to measure the drug concentrations in the major organ tissues. Any acute side effects from the drug can be evaluated using previously described methods. Specifically, serum albumin levels can be measured using human albumin ELISA kit with no cross-reactivity to mouse albumin (Bethyl Laboratories) and blood chemistry parameters can be determined, including ALT/AST and BUN/Cr. Putative liver toxicity can be assessed by histological and biochemical analysis in the treated mice. Evaluation of the on-target gene-editing and off-target effects can be determined by Sanger sequencing and NGS. The NGS sequencing data can be mapped to the human genome database and indel patterns can be analyzed by CRISPResso software. Controls can include mice without HBV infection, exosomes without PSIP (i.e., cannot target NTCP on humanized liver hepatocytes), and exosomes without RNPs (i.e., no drug for HBV gene-editing). A total of 4 groups with 48 mice (12 mice/group; 6 males and 6 females) can be used for assessing drug distribution and acute side effects. Drug administration protocol and numbers/groups of mice are shown in FIG. 9.

ix. Evaluating the Antiviral Efficacy of the Exo-HBV-Eliminator in HBV-Infected, Liver Humanized Mice

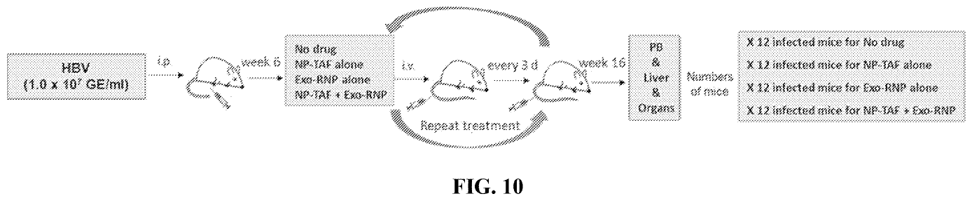

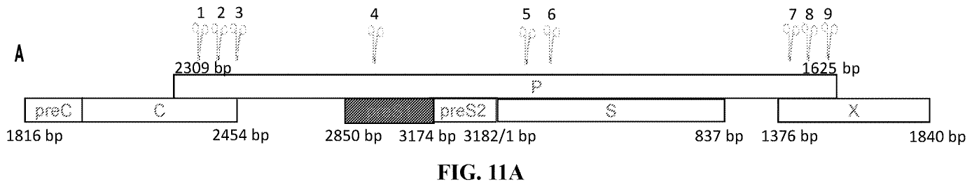

The current antiviral NA regimens can suppress HBV replication but cannot target/eliminate cccDNA to achieve an HBV cure. Thus, a combinatorial strategy, using NP-TAF plus Exo-HBV-Eliminator, can be investigated. NP-TAF can be used to reduce the amount of HBV DNA and Exo-HBV-Eliminator to eliminate HBV cccDNA from hepatocytes in HBV-infected, liver-humanized mice. HBV infection kinetics in these mice have been established using high titer (1×10⁷ GE/ml) HBV stock (FIG. 8). Thus, per the protocol proposed in FIG. 10, the Fah/NRG-hu HEP mice can be injected with HBV patient sera (1.0×10⁷ GE/ml) and then i.v. injected with the NP-TAF (10 μg/100 μl/mouse) and Exo-HBV-Eliminator drugs (0.9 nM RNPs in 100 μl vol) 162 individually or in combination at the end of week 6 post-infection. The drug treatment can be repeated every 3 days (based on the half-life of the Exo-Cas9 RNPs in the blood of NSG mice, data not shown). Mice without drug treatment and mice receiving only one drug (NP-TAF or Exo-HBV-Eliminator) can serve as controls. HBV replication/inhibition and liver function can be longitudinally evaluated weekly until week 16 (i.e., week 10 after starting the treatment; target end-point). Approximately 100-150 μl of blood can be collected via the tail-vein on day 0 and then weekly during the HBV infection and treatment (total of 16 weeks). Plasma can be isolated for the detection of HBV DNA/RNA by real-time PCR and HBsAg/HBeAg antigens by ELISA. Specifically, plasma HBV DNA can be measured weekly by purifying virion-associated DNA using QIAmp Pathogen mini kit (Qiagen), followed by real-time PCR. In addition to real-time PCR and ELISA assaying of the viral components, robust ddPCR and RNAscope techniques can be utilized to simultaneously detect HBV DNA and RNA in liver hepatocytes harvested at the end-point (week 16) or harvested from mice that may become moribund and are euthanized during the experiment. HBV cccDNA in hepatocytes can be measured as described. Chronic side effects and potential toxicity of the drugs in major organs/tissues can be determined as described above. Moreover, NGS assays can be conducted to quantify the percentage of indel formation at the predicted gRNA/Cas cleavage sites, followed by deep sequencing of the targeted HBV genes as well as the off-target regions. All assays can be run in triplicates and repeated three times independently, followed by statistical analysis. The HBV-infected mice can be randomly divided into 4 groups (12 mice/group; 6 males and 6 females) to evaluate the antiviral efficacy of the NP-TAF and Exo-HBV-Eliminator drugs. Group 1 is untreated control (drug-free); group 2 can receive NP-TAF drug alone; group 3 can receive Exo-HBV-Eliminator alone; and group 4 can receive NP-TAF drug plus Exo-HBV-Eliminator. Thus, a total of 48 mice can be. The drug administration protocol and the number/group of mice are shown in FIG. 10.

### C. Example 3: Synthetic gRNA/Cas9 Ribonucleoprotein Targeting HBV DNA Inhibits Viral Replication

### 1. Introduction

Hepatitis B virus (HBV) infection is a major public health problem, with 300 million people suffering from chronic hepatitis B (CHB) worldwide (WHO 2021). Approximately 1 million HBV-infected individuals die each year due to end-stage liver diseases, such as liver cirrhosis and hepatocellular carcinoma (HCC). While current antiviral therapy, nucleos(t)ide analogues (NAs), can suppress HBV replication, it cannot eradicate HBV infection, and NA cessation readily leads to viral reactivation and disease progression. Thus, novel therapeutic modalities are desperately needed to eliminate chronic HBV infection.

Current antiviral therapies fail to cure HBV infection, primarily due to the presence of covalently closed circular (ccc) DNA (cccDNA) in the nucleus of infected cells. Distinct from integrated HBV DNA, cccDNA is an episomal minichromosome that serves as a template for transcribing viral RNAs, including pre-genomic RNA (pgRNA), which acts as a viral transcriptional template. HBV cccDNA is very stable and cannot be directly affected by antiviral NAs. While HBV cccDNA is deemed a potential therapeutic target, there are no drugs currently available that can effectively target cccDNA. Thus, any curative strategy should include a means to eliminate the episomal cccDNA (which supports viral replication) and integrated HBV DNA (which does not support viral replication but can code HBsAg) without inducing collateral cytotoxic effects in HBV-infected cells.

Clustered regularly interspaced short palindromic repeat (CRISPR)-associated protein 9 (Cas9)-mediated genome editing is an appealing means to combat HBV infection. This approach enables the destruction of the HBV cccDNA and the cleaving of the integrated HBV DNA. Although it has shown promise in HBV gene-editing and viral suppression both in vitro and in vivo, this approach faces several challenges that need to be overcome for clinical application, e.g., its off-target effects and inefficient, non-specific in vivo delivery. Targeting specific HBV genes that are crucial for viral replication while avoiding off-target cytotoxic effects is key to the success of CRISPR/Cas9-mediated viral clearance. Additionally, the current CRISPR/Cas9 expression and/or delivery methods require viral vectors, which pose safety concerns for therapeutic application in humans. These shortcomings have largely limited the use of CRISPR/Cas9-based therapeutics for HBV eradication. Synthetic CRISPR/Cas9 can be directed to the target genes to cleave any desired DNA sequences. This can be achieved by designing a guide RNA (gRNA) with approximately 20 nucleotides that match a particular sequence of the genome with downstream protospacer adjacent motifs (PAMs). Thus, synthetic gRNA/Cas9 ribonucleoprotein (RNP) is a suitable non-viral formulation. Direct administration of synthetic gRNA/Cas9 RNPs offers versatile and transient gene-editing in a controllable way compared with other delivery methods that depend on plasmid DNA (pDNA) or messenger RNA (mRNA), which exhibit varying transcriptional and translational activities and uncontrollable expression duration in vivo, and thus can result in off-target effects and unwanted immune responses

In this study, HBV-specific target genes that are crucial for HBV replication but are not homologous (off-target) with the human genome were selected, a series of gRNA/Cas9 RNPs targeting these genes was synthesized, and their antiviral activity and cytotoxicity in HBV-infected cell models was tested. The results show that synthetic gRNA/Cas9 RNPs targeting HBV polymerase (P), surface(S), and viral enhancer and oncogenic (X) genes can efficiently inhibit HBV replication without causing any cytotoxic effects. These synthetic gRNA/Cas9-based gene-editing modalities warrant further investigation as a potential therapeutic drug for chronic HBV infection.

### 2. Materials and Methods

i. 2.1. HBV Cell Lines and Transfection with Synthetic gRNA/Cas9 RNPs

The HepG2.2.15 Human Hepatoblastoma cell line (Cat #SCC249, MilliporeSigma, Burlington, MA) is a traditional HBV cellular model that is widely used for studying HBV infection and drug development. HepG2.2.15 cells display constitutive HBV RNA transcription and DNA replication, but a lesser detection of cccDNA by Southern blot and PCR detection. The HepG2/2.2.15 cells were cultured in DMEM (Cat #MT10013CV, Fisher Scientific, Waltham, MA) supplemented with 10% Fetal bovine serum, penicillin, and streptomycin (50 ug/ml each; Fisher Scientific) and maintained at 37° C. and 5% CO₂ atmosphere. The Amaxa Cell Line Nucleofector Kit V (Lonza VCA-1003) was used to deliver gRNA/Cas9 into HepG2/2.2.15 cells stably expressing HBV. For each transfection, 160 μM gRNA and 160 μM tracrRNA were first incubated at 37° C. for 30 minutes, followed by adding 80 μM Cas9 protein and incubating at 37° C. for 15 minutes. Approximately 40 μM of the gRNA/Cas9 complex was transfected into HepG2/2.2.15 cells (5×10⁵) following the manufacturer's instructions. After 72 hours, the culture supernatants were harvested to measure HBV antigens by ELISA, and the cell pellets were used to determine HBV DNA or mRNA levels by real-time-PCR.

The HepDE19 cell line is tetracycline-controlled HBV stable cells maintained in the presence of 500 μg/ml G418 and 1 μg/ml tetracycline (Tet). Upon withdrawing Tet from the culture fluid of HepDE19 cells, pgRNA transcription, HBV DNA replication, cccDNA formation, and HBV e antigen (HBeAg) production gradually increase in a time-dependent manner. To induce HBV replication, the cells were cultured in a Tet-free medium for 0-10 days, followed by measuring the expression of HBV antigens, HBV cccDNA, and pgRNA using methods previously described. For cell transfection, HepDE19 cells were cultured in Tet-free medium for 2 days. The gRNA/Cas9 RNPs and Lipofectamine CRISPRMAX reagents were prepared in two separate tubes following the manufacturer's instructions. Briefly, 125 ng gRNA, 500 ng GeneArt Platinum Cas9 nuclease, and 1 μl Cas9 Plus reagents were mixed in one tube, and 1.5 μl Lipofectamine CRISPRMAX reagent was added in a separate tube. The gRNA/Cas9 RNP solution was then transferred to the tube containing the Lipofectamine CRISPRMAX solution. The mixture was vortexed and incubated at 25° C. for 10 min to form the gRNA/Cas9 RNP/Lipofectamine CRISPRMAX complex. Meanwhile, HepDE19 cells were seeded in a 24-well plate at 2.0×10⁵ cells per well. The gRNA/Cas9 RNP/Lipofectamine CRISPRMAX complex was then added directly to the cell suspension, and the cells were incubated for 72 h, followed by assessing HBV replication with Tet-free medium as described above.

ii. Infection of HepG2-NTCP Cells with HBV and Transfection with Synthetic gRNA/Cas9 RNPs

HBV infectious particles were harvested from the supernatants of gRNA/Cas9-treated HepDE19 cells and used for the infection of HepG2-NTCP cells following a previously published method. Briefly, the supernatants of gRNA RNP-treated HepDE19 cells were harvested and concentrated using a PEG concentration kit (Cat #ab102538, Abcam, Waltham, MA). For HBV infection, 2.5×10⁵ HepG2-NTCP cells were seeded in a 12-well plate and mixed with 10 μl concentrated HBV supernatants in 1 ml hepatocyte maintenance medium (HMM) (with 8% PEG8000 and 5% DMSO) at 37° C. for 16 h. The culture media were changed daily with fresh HMM. After 8 days, the cells were harvested and HBV DNA and cccDNA levels were determined as described below.

In another experiment, 1×10⁶ HepG2-NTCP cells were infected using supernatants containing HBV particles from HepDE19 cells that were maintained for 6 days. The HBV-infected HepG2-NTCP cells were transfected with gRNA5, gRNA9, gRNA5 plus gRNA9, or gRNAc RNPs as described above. The cells were maintained for 3 days in a complete culture medium and then harvested for HBV cccDNA detection by qPCR.

iii. HBsAg and HBeAg ELISA

The levels of HBsAg and HBeAg in the supernatants of cell cultures were determined by ELISA (Cat #KA0286 and NBP2-600029, Novus Biologicals, Littleton, CO) according to the manufacturer's protocol. The optical density of antigen levels was measured at 450 nm using a microplate spectrophotometer. The samples were run in triplicate, and the experiments were repeated at least three times.

iv. HBV mRNA qPCR

Total RNA was extracted from the cell pellets by the RNeasy Mini kit (Cat #74104, QIAGEN, Germantown, MD), and 1 μg RNA was converted to cDNA using the Applied Biosystems High-Capacity cDNA Reverse Transcription Kit (Cat #43-688-14, ThermoFisher, Waltham, MA). Quantitative real-time PCR (real-time-qPCR) was performed using iTaq Universal SYBR Green Supermix (Cat #1725124, Bio-Rad, Hercules, CA). The PCR primers were: HBV mRNA forward, 5′-GAGTGCTGTATGGTGAGGTG-3′ (SEQ ID NO: 52), reverse, 5′-TTTGGGGCATGGACATTGAC-3′ (SEQ ID NO: 53); HBV pgRNA forward 5′-CTCCTCCAGCTTATAGACC-3′ (SEQ ID NO: 54), reverse 5′-GTGAGTGGGCCTACAAA-3′ (SEQ ID NO: 55); and GAPDH forward 5′-ACAACTTTGGTATCGTGGAAGG-3′ (SEQ ID NO: 56), reverse 5′-GCCATCACGCCACAGTTTC-3′ (SEQ ID NO: 57). The gene expression levels were determined by the 2-44Cq method and values were normalized to GAPDH expression as an internal control.

v. HBV DNA and cccDNA qPCR

Total and protein-free HBV DNAs were extracted from whole cell lysate using the Hirt DNA method according to a previously published protocol. For HBV cccDNA analysis, the protein-free DNA was treated with 10 units of T5 exonuclease (NEB) at 37° C. for 30 min, followed by 95° C. for 5 min, and diluted 4-fold with nuclease-free water. Next, the cccDNA was cleaned with a DNA clean and concentrator kit (ZYMO Research, Irvine, CA) and used for PCR. HBV total DNA and cccDNA were analyzed by real-time PCR using a TaqMan PCR assay. The primers and probe used to amplify HBV total DNA were: HBV total DNA forward: 5′-CCGTCTGTGCCTTCTCATCTG-3′ (SEQ ID NO: 58), reverse 5′-AGTCCAAGAGTYCTCTTATGYAAGACCTT-3′ (SEQ ID NO: 59), and internal probe: 5′-FAM-CCGTGTGCACTTCGCTTCACCTCTGC-TAMRA-3′ (SEQ ID NO: 60). The primers and probe used to amplify HBV cccDNA (Genotype D, subtype ayw) were: forward: 5′-TCATCTGCCGGACCGTGTGC-3′ (SEQ ID NO: 61), reverse 5′-TCCCGATACAGAGCTGAGGCGG-3′ (SEQ ID NO: 62), and internal probe 5′-FAM-TTCAAGCCTCCAAGCTGTGCCTTGGGTGGC-TAMRA-3′ (SEQ ID NO: 63). The PCR reactions were performed in 20 μl volumes and contained 2 μl DNA. The PCR conditions were: 95° C. for 10 min, followed by 45 cycles of 95° C. for 15 s and 64° C. for 30 s. The DNA levels were determined by the 2-44Cq method and values were normalized to the control group.

vi. HBV RNA Detection by RNAscope:

RNAscope was performed using an assay kit from ACD (Newark, CA). Briefly, the cells were cultured and attached on cover glasses (8 mm in diameter, FisherSci) in a 24-well plate overnight, followed by fixation with 10% Neutral Buffered Formalin (NBF), dehydration, immobilization. Next, the slides were incubated with RNAscope hydrogen peroxide at room temperature (RT) for 10 min, followed by incubating with RNAscope Protease III diluted 1:5 with 1× PBS at RT for 10 min. Then, the RNAscope probe was hybridized at 40° C. for 2 h in a hybridization oven, then processed with RNAscope 2.0 HD detection reagent. Finally, the slides were dried in a 60° C. oven, briefly rinsed with pure xylene, and immediately covered with 1 drop of EcoMount (Fisher Scientific) by a coverslip (40×24 mm, FisherSci). Images were obtained using an EVOS microscope (Life Technologies, Carlsbad, CA) and analyzed by QuPath software and GraphPad Prism 7 (Irvine, CA).

vii. HBV DNA Mutagenesis Determination by T7E1 Assay

The HBV DNA sequences surrounding gRNA-binding sites were amplified by PCR. The PCR primers used for detecting the gRNA5 flanking region were: forward 5′-GACAAGAATCCTCACAATA-3′ (SEQ ID NO: 64), reverse 5′-CATAGAGGTTCCTTGAGCAG-3′ ((SEQ ID NO: 65). The primers used for detecting the gRNA9 flanking region were: forward 5′-TACATCGTTTCCATGGCTGCTAG-3′ (SEQ ID NO: 66), reverse: 5′-CAACTCCTCCCAGTCTTTAAAC-3′ (SEQ ID NO: 67). All PCR products were verified by Sanger sequencing and subjected to a re-annealing process to induce heteroduplex formation. After re-annealing, the PCR products were treated with T7E1 (New England Biolabs, Ipswich, MA) at 37° C. for 30 min and analyzed using 3% agarose gels. The gel band images were acquired using a gel imaging system (Bio-Rad).

### VIII. Hbv DNA Sequencing.

Following treatment with gRNA5/Cas9+gRNA9/Cas9, or gRNAc/Cas9 RNPs, genomic DNA was isolated from HepDE19 cells using the PureLink Genomic DNA Mini Kit (Cat #K182001, Invitrogen). DNA fragments containing the gRNA target sequences were amplified by qPCR using primers flanking the cleavage sites. The PCR products were confirmed using 1% agarose gel electrophoresis and purified using a QIAquick gel extraction kit (Cat #28704, Qiagen). Sequencing of the extracted DNA fragments was performed by GENEWIZ (Chelmsford, MA) using Sanger's sequencing method. The PCR primers used for the detection of gRNA5 and gRNA9 flanking regions are described above. The sequencing readouts were aligned with the gRNAc, which was compared to the HBV sequence in the database (ayw strain, Genbank accession number: NC_003977.2). The Chromas's software (Technelysium DNA sequencing software) was used to read the nucleotide peaks, and the alignment of two or more sequences was performed using the BLAST tool from NCBI-BLAST to compare the nucleotide sequences.

ix. Cytotoxicity Assay:

The MTT assay was used to determine metabolic activity and cell viability as an indicator of cellular cytotoxicity. Twenty-four hours after gRNA/Cas9 transfection, the cells were cultured in a Tet-free medium for an additional 24 h. For measuring cell proliferation, the cells were cultured in the Tet-free medium for an additional 3 days in a 96-well plate. The culture medium (100 μl) was changed and MTT reagent (0.5 mg/ml) was added at 37° C. for 4 h, followed by measuring the absorbance at 550-690 nm using a spectrophotometer (BioTek SYNERGY H1).

x. Statistical Analysis:

All data were analyzed using Prism 7 software and are expressed as mean±SE. Comparisons between two groups were made using a parametric paired or unpaired t-test for normally distributed data or a non-parametric Wilcoxon paired t-test or Mann-Whitney U-test for non-normal distributions. Comparisons among multiple groups were made using a one-way ANOVA at a 95% confidence level (Tukey's honest significance test). P-values <0.05 (*) were considered statistically significant and p-values <0.01 (**), <0.001 (***), or <0.0001 (****) were considered very significant.

### 3. Results

i. Design and Synthesis of gRNA/Cas9 RNPs Targeting HBV Genomes:

Identifying gRNAs targeting specific HBV genes with minimal predicted off-target effects is the first step in designing a CRISPR/Cas9 RNP for HBV treatment. The major challenge of selecting these specific targets on HBV DNA is the high heterogeneity of the HBV genome (i.e., HBV quasispecies), complicating the design of gRNAs that would target multiple or all HBV genotypes (A-H). Thus, identifying new gRNAs for specific Cas9 targeting highly conserved regions of HBV DNA is essential for the CRISPR/Cas9 design to move into clinical application for HBV eradication.

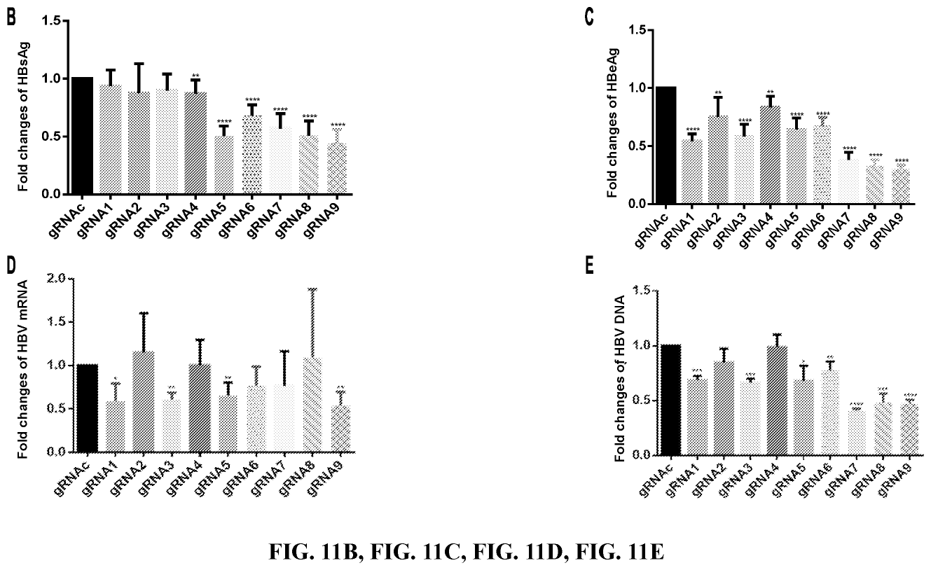

To select the most specific and potent targeting sites within the HBV genome, e-CRISP online gRNA designing tool was used to compare HBV sequences for potential HBV gRNA targeting sites that have the best-predicted on-target (i.e., HBV sequences that are critical for viral replication) and lowest off-target effects (i.e., 0% homology with the human genome). The genomic DNA sequences of 8 HBV genotypes (A-H) originating from distinct regions were compared and 9 target sites in highly conserved regions on the HBV genome were identified, including polymerase (P), capsid (C), surface(S), and X genes (FIG. 11A). gRNAs were synthesized based on the ayw strain (Genbank accession number: NC_003977.2), and these gRNAs carry an NGG codon (where N can be any nucleotide) at the 3′-end following the HBV sequence complementary to the protospacer adjacent motif (PAM), which can be recognized by the Cas9 nuclear localization signal (NLS) that can direct gRNA/cas9 RNP into the cell nucleus. The features of these selected gRNAs, including their name, lengths, start/end positions, orientation (plus/minus strands), nucleotide sequences, % of ATGC nucleotides, seed GC contents, S-/E-/Doench-scores, and numbers of mismatch hits, are presented in Table 1. The on-target antiviral activity and off-target cytotoxic effects of these 9 HBV-specific gRNA/Cas9 RNPs were tested and compared with a gRNA using different HBV-infected cell model systems.

TABLE 4
Features of gRNAs designed and tested in this study

| | HBV | HBV | HBV |
|---|---|---|---|
| Name | gRNA 1 | gRNA 2 | gRNA 3 |
| Length | 23 | 23 | 23 |
| Start | 2318 | 2342 | 2346 |
| End | 2341 | 2365 | 2369 |
| Strand | minus | minus | plus |
| Nucleotide | GATTGA | GATTGA | GTCGCA |
| sequence | GACCTT | GATCTT | GAAGAT |
| | CGTCTG | CTGCGA | CTCAAT |
| | CG NGG | CG NGG | CT NGG |
| Gene | NA | NA | NA |
| Name | | | |
| Transcripts | NA | NA | NA |
| Transcript:: | NA | NA | NA |
| Exon | | | |
| Number of | NA | NA | NA |
| Cpg | | | |
| Islands | | | |
| hit | | | |
| Sequence | NA | NA | NA |
| around | | | |
| the | | | |
| cutside | | | |
| % A | 24 36 16 | 24 36 20 | 28 24 20 |
| % C | 24 | 20 | 28 |
| % T | | | |
| % G | | | |
| S- | 40 | 60 | 80 |
| Score | | | |
| A- | 0 | 0 | 0 |
| Score | | | |
| E- | 68.3874 | 80.6503 | 53.3841 |
| Score | | | |
| percent | NA | NA | NA |
| of | | | |
| total | | | |
| transcripts | | | |
| hit | | | |
| Target | * | * | * |
| Match- | 0 | 0 | 0 |
| start | | | |
| Match- | 0 | 0 | 0 |
| end | | | |
| Matchstring | NA | NA | NA |
| Edit | 0 | 0 | 0 |
| distance | | | |
| Number of | 4 | 3 | 2 |
| Hits | | | |
| Direction | NA | NA | NA |
| CDS_score | 0 | 0 | 0 |
| Exon_Score | 0 | 0 | 0 |
| seed_GC | 0.7 | 0.8 | 0.5 |
| Doench_Score | 0.537490841 | 0.799378984 | 0.04104376 |
| Xu_score | 0.181883387 | 0.433139128 | 0.128164786 |
| Chromosome | NA | NA | NA |
| Genomic | 1818 | 1842 | 1846 |
| start | | | |
| Genomic | 1841 | 1865 | 1869 |
| End | | | |
| Name | HBV | HBV | HBV |
| Length | gRNA 4 | gRNA 5 | gRNA 6 |
| Start | 23 | 23 | 23 |
| End | 2902 | 297 | 349 |
| Strand | 2925 | 320 | 372 |
| Nucleotide | minus | Plus | plus |
| sequence | GCTCCT | GTTATC | GCTATG |
| | ACCTTG | GCTGG | CCTCAT |
| | TTGGCG | ATGTGT | CTTCTT |
| | TC NGG | CTG | GT NGG |
| | | NGG | |
| Gene | NA | NA | NA |
| Name | | | |
| Transcripts | NA | NA | NA |
| Transcript:: | NA | NA | NA |
| Exon | | | |
| Number of | NA | NA | NA |
| Cpg | | | |
| Islands | | | |
| hit | | | |
| Sequence | NA | NA | NA |
| around | | | |
| the | | | |
| cutside | | | |
| % A | 32 28 8 | 8 20 32 | 8 24 48 |
| % C | 32 | 40 | 20 |
| % T | | | |
| % G | | | |
| S- | 80 | 100 | 100 |
| Score | | | |
| A- | 0 | 0 | 0 |
| Score | | | |
| E- | 54.3242 | 61.7668 | 56.9203 |
| Score | | | |
| percent | NA | NA | NA |
| of | | | |
| total | | | |
| transcripts | | | |
| hit | | | |
| Target | * | * | * |
| Match- | 0 | 0 | 0 |
| start | | | |
| Match- | 0 | 0 | 0 |
| end | | | |
| Matchstring | NA | NA | NA |
| Edit | 0 | 0 | 0 |
| distance | | | |
| Number of | 2 | 1 | 1 |
| Hits | | | |
| Direction | NA | NA | NA |
| CDS_score | 0 | 0 | 0 |
| Exon_Score | 0 | 0 | 0 |
| seed_GC | 0.6 | 0.6 | 0.4 |
| Doench_Score | 0.115975383 | 0.303114158 | 0.464274315 |
| Xu_score | 0.000235774 | 0.18522702 | -0.018258996 |
| Chromosome | NA | NA | NA |
| Genomic | 2402 | -203 | -151 |
| start | | | |
| Genomic | 2425 | -180 | -128 |
| End | | | |
| | HBV | HBV | HBV |
| Name | gRNA 7 | gRNA 8 | gRNA 9 |
| Length | 23 | 23 | 23 |
| Start | 1422 | 1501 | 1525 |
| End | 1445 | 1524 | 1548 |
| Strand | minus | minus | minus |
| Nucleotide | GTCGGA | GAAGCG | GGTCTC |
| sequence | ACGGCA | AAGTGC | CATGCG |
| | GACGGA | ACACGG | ACGTGC |
| | GA NGG | TC NGG | AG NGG |
| Gene | NA | NA | NA |
| Name | | | |
| Transcripts | NA | NA | NA |
| Transcript:: | NA | NA | NA |
| Exon | | | |
| Number of | NA | NA | NA |
| Cpg | | | |
| Islands | | | |
| hit | | | |
| Sequence | NA | NA | NA |
| around | | | |
| the | | | |
| cutside | | | |
| % A | 4 48 28 20 | 12 40 24 | 20 40 16 |
| % C | | 24 | 24 |
| % T | | | |
| % G | | | |
| S- | 60 | 60 | 80 |
| Score | | | |
| A- | 0 | 0 | 0 |
| Score | | | |
| E- | 60.2608 | 61.6696 | 67.6774 |
| Score | | | |
| percent | NA | NA | NA |
| of | | | |
| total | | | |
| transcripts | | | |
| hit | | | |
| Target | * | * | * |
| Match- | 0 | 0 | 0 |
| start | | | |
| Match- | 0 | 0 | 0 |
| end | | | |
| Matchstring | NA | NA | NA |
| Edit | 0 | 0 | 0 |
| distance | | | |
| Number of | 3 | 3 | 2 |
| Hits | | | |
| Direction | NA | NA | NA |
| CDS_score | 0 | 0 | 0 |
| Exon_Score | 0 | 0 | 0 |
| seed_GC | 0.6 | 0.7 | 0.6 |
| Doench_Score | 0.021491185 | 0.085875578 | 0.305634687 |
| Xu_score | 0.391550943 | 0.297604811 | 0.478239533 |
| Chromosome | NA | NA | NA |
| Genomic | 922 | 1001 | 1025 |
| start | | | |
| Genomic | 945 | 1024 | 1048 |
| End | | | |

ii. Synthetic gRNA/Cas9 RNPs Inhibit HBV Replication in HepG2/2.2.15 Cells

Because the hepatoma cell line HepG2/2.2.15 is a traditional HBV cellular model used to test the efficacy and cytotoxicity of anti-HBV drugs, the synthetic gRNA/Cas9 RNPs were tested for their antiviral efficiency in HepG2/2.2.15 cells. After 3 days of nucleofection, HBsAg and HBeAg levels were measured in the supernatants of the treated cells. As shown in FIG. 11B, HBsAg levels significantly decreased in cells transfected with gRNA4-9/Cas9 compared with the scrambled gRNAc. In addition, transfection with gRNA5, gRNA8, or gRNA9 led to a 50% reduction in HBsAg expression. The levels of HBeAg were also reduced in all gRNA/Cas9-transfected cells, with >50% reduction after treatment with gRNA7, gRNA8, or gRNA9 compared with the gRNAc (FIG. 11C). Next, total RNA from HepG2/2.2.15 cells transfected with gRNA/Cas9 was isolated and the relative HBV mRNA expression levels were determined by real-time PCR. The results showed a remarkable suppression (~50% reduction) of HBV mRNA expression in cells transfected with gRNA1, gRNA3, gRNA5, or gRNA9 compared with gRNAc (FIG. 11D). Correspondingly, a significant reduction in the HBV DNA level in the transfected cells was observed, with gRNA7, gRNA8, or gRNA9 transfection showing >50% reduction compared with the gRNAc transfection (FIG. 11E). The gRNAs were designed to target different region of HBV genome and thus may elicit different antiviral effects on the expression of HBV antigens, as well as the levels of HBV mRNA and HBV DNA. The differences in these virologic readouts in the treated cells may also result from varying sensitivity of the detection methods. Because the transfection efficiency in the HepG2-based cell line was ~50%, a 50% reduction in these virologic readouts represents a remarkable antiviral effect in this HBV-infected cell model. Based on their inhibitory efficacy and specific targeting sites, gRNA5 (which targets HBV P/S genes) and gRNA9 (which targets HBV P/X genes) were chosen for further testing using an advanced HBV-infected cell model.

iii. Synthetic gRNA/Cas9 RNPs Inhibit HBV Replication in HepDE19 Cells:

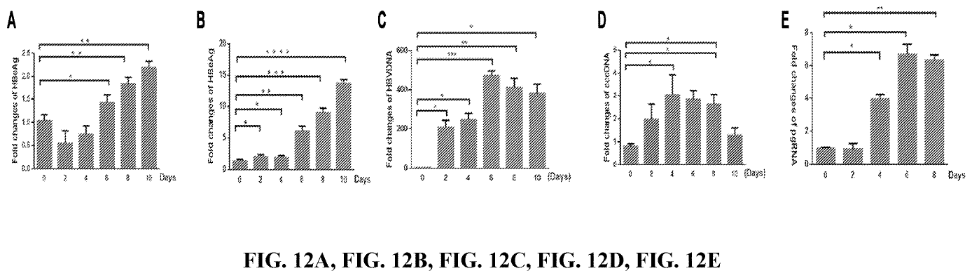

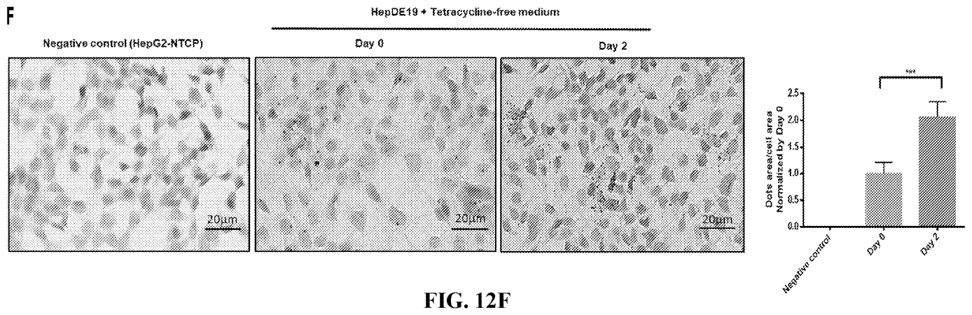

The HepDE19 cell line is a robust HBV cell culture system that can support HBV replication and propagation. This cell line was maintained in the presence of 1 μg/ml tetracycline (Tet) and G418 (500 μg/ml). In this stable cell line, HBV replication is under the control of a Tet responsive promoter, which results in a controllable production of HBV compared with the HepG2.2.15 cell line, and the expression of pgRNA, cccDNA, and HBeAg are induced when the cells are cultured in Tet-free medium (for viral induction). As shown in FIG. 12, HepDE19 cells exhibited a time-dependent increase in the expression of HBsAg (FIG. 12A) and HBeAg (FIG. 12B), as determined in the culture supernatants by ELISA. In addition, real-time PCR revealed a time-dependent increase in total HBV DNA (FIG. 12C), cccDNA (FIG. 12D), and pgRNA (FIG. 12E) in HepDE19 cells cultured with Tet-free medium. Increases in HBV RNA expression were also detected by RNAscope in HepDE19 cells at day 0 and day 2 after incubation in Tet-free medium (FIG. 12F). With these detectable HBV readouts, this cell line can serve as a reliable model to test the efficacy of antiviral drugs.

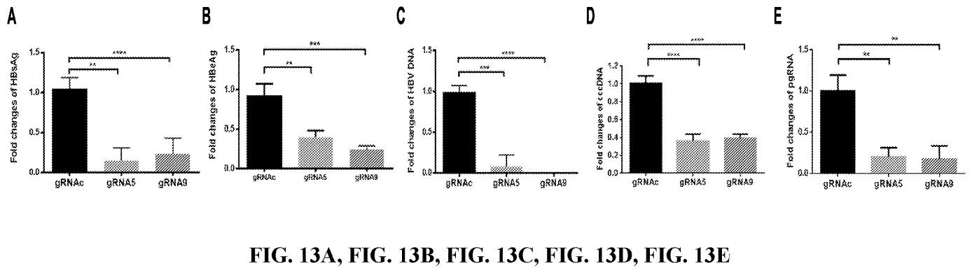

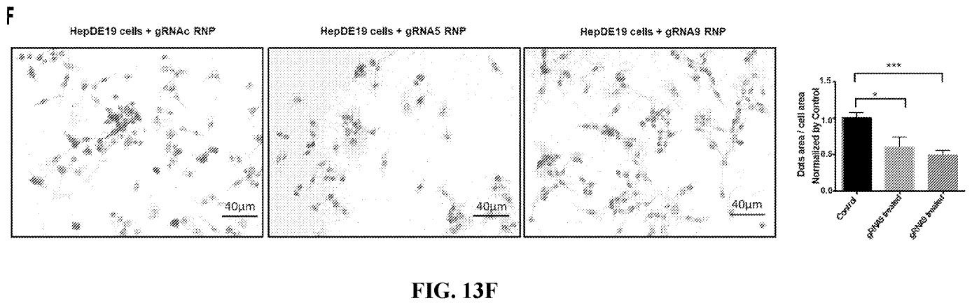

Next, the antiviral efficacy of the synthetic gRNA5 and gRNA9 in HepDE19 cells was tested. As shown in FIG. 13, HBV replication was significantly suppressed in HepDE19 cells transfected with gRNA5 or gRNA9 compared with the scrambled gRNAc, as evidenced by the significant decreases in the expression of HBsAg (FIG. 13A), HBeAg (FIG. 13B), total HBV DNA (FIG. 13C), cccDNA (FIG. 13D), pgRNA (FIG. 13E), and HBV RNA (FIG. 13F) following incubation with Tet-free medium for 3 days. Taken together, these results demonstrate that gRNA5 or gRNA9 can significantly suppress HBV replication in HBV-infected cells.

iv. HepDE19 Cells and HepG2-NTCP Cells Transfected with gRNA/Cas9 RNPs Produce Fewer Infectious HBV Particles

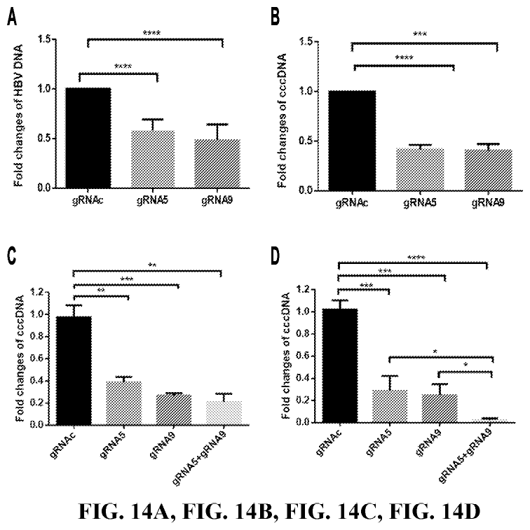

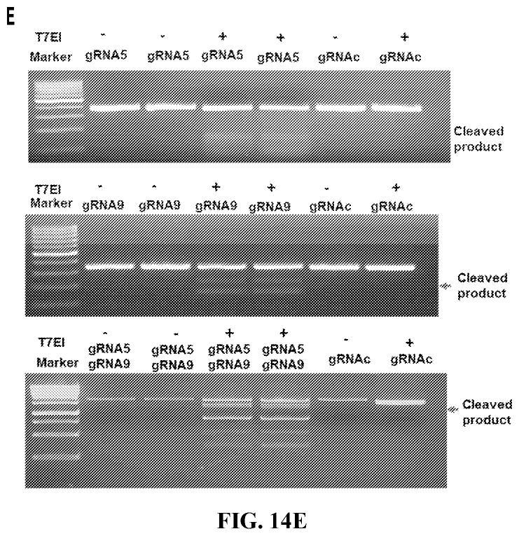

Sodium taurocholate cotransporting polypeptide (NTCP) is a functional receptor for human HBV. The human hepatoma cell line HepG2 is not susceptible to HBV infection due to lack of NTCP; however, HepG2 cells transfected with NTCP expression plasmids (HepG2-NTCP) are susceptible to natural HBV infection. To further characterize the inhibitory effects of the synthetic gRNA/Cas9 RNPs on HBV replication, HepG2-NTCP cells were incubated with supernatants collected from gRNA/Cas9 RNP-transfected HepDE19 cell culture (after 4 days). Genomic DNA was isolated from HepG2-NTCP cells on day 8 after HBV infection and used for the assessment of HBV replication. Compared with cells infected with supernatants from the gRNAc-treated cells, HepG2-NTCP cells infected with the supernatants of gRNA5- and gRNA9-treated HepDE19 cells had significantly lower HBV DNA levels (FIG. 14A). Moreover, the amount of HBV cccDNA was also remarkably lower in HepG2-NTCP cells infected with the supernatants of gRNA5- and gRNA9-transfected HepDE19 cells (FIG. 14B). These results indicate that gRNA5- or gRNA9-treated HepDE19 cells produce fewer copies of infectious HBV particles capable of infecting HepG2-NTCP cells.

To further characterize the anti-HBV activities of gRNA5 and gRNA9 RNPs in newly infected cells, HBV particles derived from HepDE19 cells were used to infect HepG2-NTCP cells, followed by transfecting the newly HBV-infected HepG2-NTCP cells. It was found that transfection with gRNA5, gRNA9, or gRNA5 plus gRNA9 significantly reduced HBV cccDNA levels (FIG. 14C).

Next, it was sought to determine whether repeated treatments with gRNA/Cas9 can increase the antiviral efficacy in HepDE19 cells with stable HBV infection. To this end, HepDE19 cells cultured with Tet-free medium for 2 days were transfected with gRNA5, gRNA9, or gRNA5 plus gRNA9 every 3 days for a total of four treatment. After four treatments, gRNA5 or gRNA9 alone elicited ~70-80% reduction in HBV cccDNA compared with the scrambled gRNAc treatment (FIG. 14D). Unexpectedly, four treatments with gRNA5 or gRNA9 alone did not increase the antiviral efficacy more than the levels observed with one-time treatment (FIG. 13D). However, four-times treatments with gRNA5 plus gRNA9 elicited a significant reduction (>95%) in HBV cccDNA levels compared with the scrambled gRNAc (FIG. 14D). Taken together, these results indicate that gRNA5 and gRNA9 are good candidates for HBV drug development.

v. Cleavage of HBV DNA by Synthetic gRNA/Cas9 RNPs in HepDE19 Cells

DNA cleavage by CRISPR/Cas9 results in double-strand breaks (DSBs), which are primarily repaired by the non-homologous end joining (NHEJ) repair pathway, resulting in gene mutations. Thus, detecting DNA cleavage and mismatch mutations following CRISPR/Cas9-mediated DNA damage repair is important for confirming gene-editing effects. To assess the capacity of the gRNA/Cas9 RNP candidates to induce cleavages in HBV DNA in HepDE19 cells, a mismatch sensitive T7 endonuclease 1 (T7E1) assay was performed, following transfection of HepDE19 cells with gRNA5, gRNA9, gRNA5 plus gRNA9, or gRNAc RNPs for 3 days. This assay allows for the detection of specific gene cleavage or mutations in HBV DNA in the treated cells. The mutagenesis was determined based on the DNA fragments detected after the gRNA/Cas9-mediated cleavage of the HBV DNA following T7E1 digestion. As shown in FIG. 14E, DSBs were introduced in HBV DNA by T7E1 treatment following HepDE19 cell transfection with gRNA5 (upper panel), gRNA9 (middle panel), and gRNA5 plus gRNA9 (lower panel), but not in the gRNAc-transfected cells or in cells without T7E1 treatment. These results confirm that gRNA5, gRNA9, or gRNA5 plus gRNA9 can induce cleavages and mutations in targeted HBV DNA.

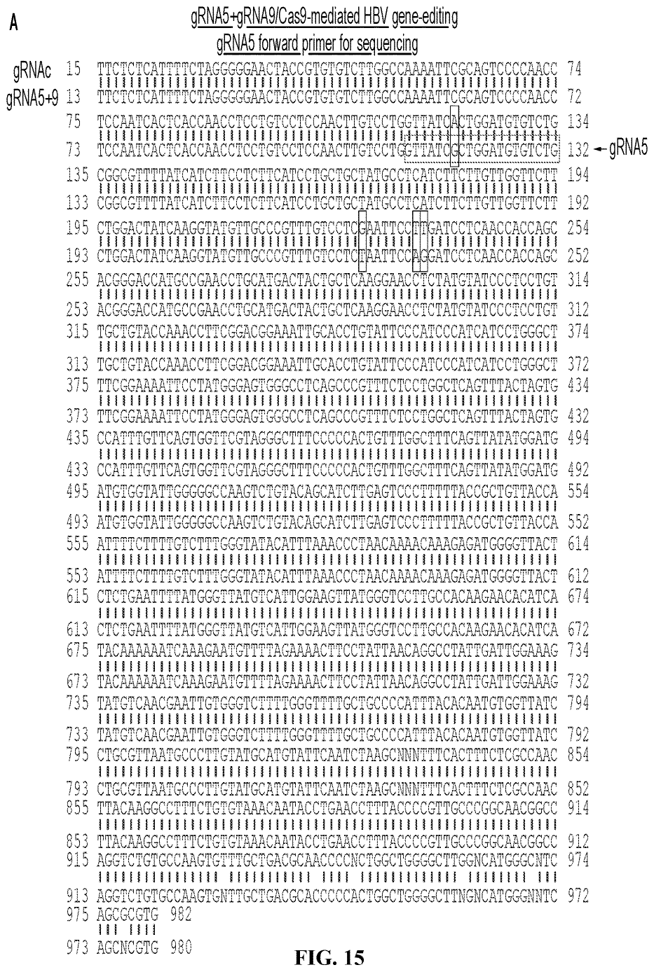

Following a successful CRISPR/Cas9 cleavage, DNA damage repair pathways are initiated, and nucleotide mutagenesis occurs in the targeted genome, introducing substitution mutations. To confirm mutations in the edited (damaged/repaired) HBV DNA, Sanger sequencing was performed after PCR amplification of the HBV DNA fragments containing the gRNA target sites from the gRNA5 plus gRNA9-treated HepDE19 cells. Compared to the treatment with gRNAc, which has a sequence identical to the HBV DNA sequence in the database (ayw strain, Genbank accession number: NC_003977.2), multiple mismatch substitution (red boxed) mutations were observed at and/or around the gRNA5 and gRNA9 target sites in treated cells (FIG. 15). These results confirm the targeted mutations within HBV genes in HepDE19 cells treated with gRNA5 and gRNA9 RNPs.

vi. Cytotoxicity of gRNA/Cas9 RNP in HepDE19 Cells

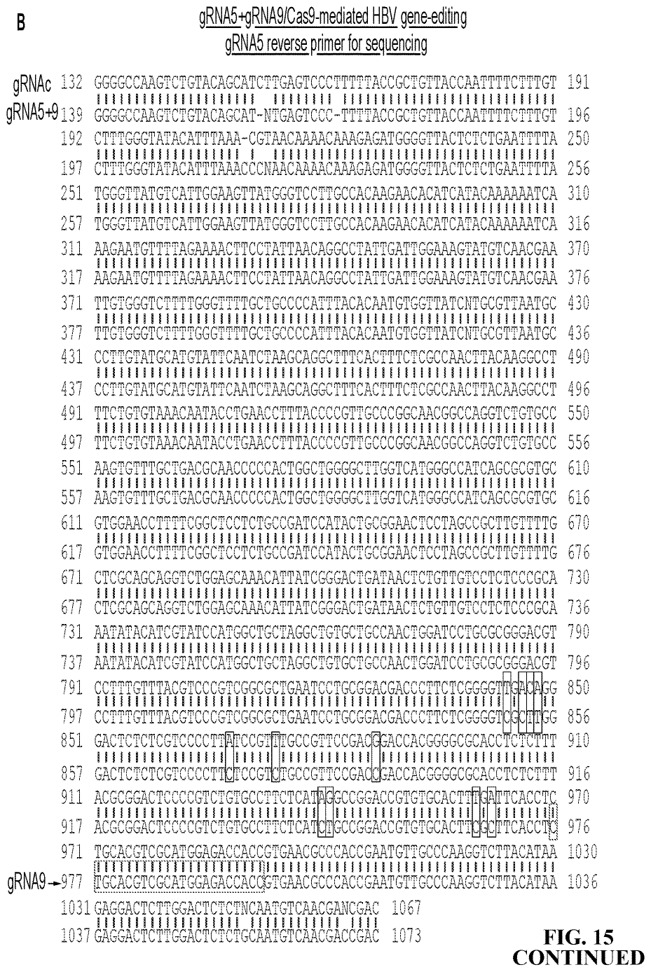

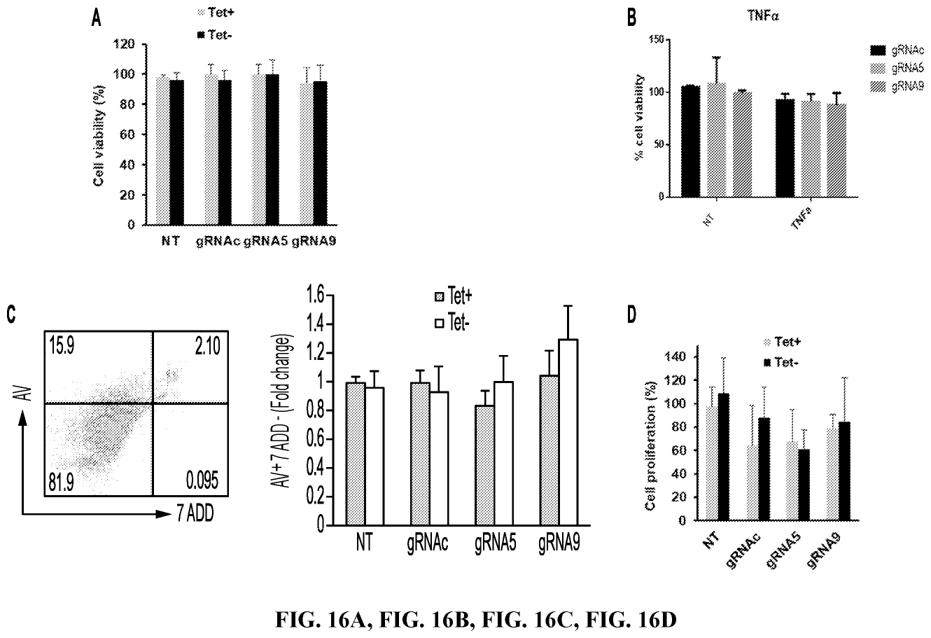

NAD (P) H-dependent cellular oxidoreductase enzymes reduce the tetrazolium MTT (3-(4,5-Dimethylthiazol 2-yl)-2,5-diphenyltetrazolium bromide) salt into insoluble formazan, which indicates the overall survival and metabolic activity of a living cell. To determine whether gRNA5/Cas9 or gRNA9/Cas9 exert any cytotoxic effects on HepDE19 cells, cell viability after transfection with gRNA5 or gRNA9 in the presence or absence of Tet-free medium (for HBV induction) for 24 h was measured using the MTT assay. As shown in FIG. 16A, compared with the non-transfected (NT) control, no significant differences in the viability of the gRNA5- or gRNA9-transfected HepDE19 cells were observed. Since HBV itself is not a direct cytopathic pathogen but can induce cell injury via immune-mediated mechanisms, the viability of transfected and non-transfected HepDE19 cells cultured in a Tet-free medium in the presence of TNFα, which can induce apoptotic cell death, was observed. No cytotoxic effects from gRNA5 or gRNA9 treatment compared with the gRNAc or control were observed (FIG. 16B). Moreover, flow cytometry analysis was performed to detect the cell surface apoptotic marker annexin V and the loss of the plasma membrane integrity (measured by up taking 7-ADD). Cell apoptosis was then evaluated by measuring the Av and 7ADD levels in gRNA-transfected HepDE19 cells with or without Tet-free medium. No significant differences in cell apoptosis among the different treatments were observed (FIG. 16C). Finally, the proliferative ability of HepDE19 cells with or without gRNA transfection for 3 days was assessed, using the MTT assay. No significant differences in cell proliferation were found (FIG. 16D). Taken together, these results indicate that the synthetic gRNA5/Cas9 and gRNA9/Cas9 RNPs are not cytotoxic to human hepatocytes.

### 4. Discussion

CRISPR/Cas9 can induce sequence-specific cleavage or mutation in HBV DNA that is integrated into the host cell genome and disrupt HBV cccDNA. The use of CRISPR/Cas9 is currently the best method for the functional inhibition of HBV cccDNA; however, its off-target effects and delivery by viral vectors create safety concerns in human applications. In the present study, synthetic gRNA/Cas9 RNPs were used to develop CRISPR/Cas9-mediated HBV gene-editing drugs. A series of gRNAs targeting different HBV genes crucial for HBV replication were designed and tested for their antiviral efficacy and cellular cytotoxicity in stably infected HBV cell lines, Hep2.2.15 and HepDE19, as well as in HBV-infected hepatoma cells expressing HBV receptor (HepG2-NTCP). It was demonstrated that these synthetic HBV gRNA/Cas9 RNPs can efficiently suppress HBV replication, as evidenced by the significant reduction in HBV DNA, cccDNA, pgRNA, mRNA, and HBsAg as well as HBeAg levels in the stably HBV-producing cell lines and in newly HBV-infected HepG2-NTCP cells. While the molecular mechanisms underlying this viral suppression remain unclear, these drugs likely induce rapid cleavage or mutation in HBV DNA, which was confirmed by the mismatch cleavage assay and Sanger DNA sequencing, resulting in a high percentage of DNA damage that cannot be fully repaired by the host cell DNA repair machinery. These findings indicate that the synthetic gRNA/Cas9 RNPs can be employed as a potential HBV DNA-editing drug to combat chronic HBV infection.

HBV cccDNA serves as a template for viral pgRNA/mRNA synthesis and is resistant to antiviral NA treatment in patients with chronic HBV infection. Here, it was demonstrated that the specifically designed and selected gRNA (gRNA5 and/or gRNA9)/Cas9 RNPs, individually or in combination, can significantly reduce HBV cccDNA levels in stably HBV-transfected cells. Specifically, the results showed that one-time or repeated (4-times) transfection of HepDE19 cells with gRNA5, gRNA9, or gRNA5 plus gRNA9 RNPs can reduce HBV cccDNA by ~70-80% (with a single gRNA transfection) and >95% (with repeated transfection of gRNA5 plus gRNA9) compared with the control gRNAc transfection (FIG. 13D, FIG. 14D). Interestingly, while repeated transfection with a single gRNA (gRNA5 or gRNA9) reduced HBV cccDNA to a level similar to one-time transfection, repeated transfection with gRNA5 plus gRNA9 RNPs combined (i.e., dual DNA excision) led to a more pronounced (>95%) reduction in HBV cccDNA (FIG. 14D). T7E1 cleavage assay and Sanger DNA sequencing confirmed specific HBV DNA gene-editing events (mutations) at or near the sites targeted by gRNA5 and gRNA9. It should be noted that upon repeating the DNA sequencing several times, it was observed that verifying DNA mutations using Sanger sequencing can be tricky since the CRISPR/Cas9-mediated gene-editing (mutations after DNA damage and repair) does not always modify the target genes exactly at the expected sites and with alterations in the same nucleotides. The gene-editing process often introduces random mutations (insertion, deletion, or substitution) at or around the gRNA targeting (binding) sites, where Cas9 nuclease protein-generated DNA damages are repaired by the NHEJ pathway. Because this DNA damage and repair process is a biological event that takes place within the treated cells, it can result in different nucleotide mutations in different batches of cells and generate a mixture of DNA products, despite applying the same treatment (with gRNA/Cas9 transfection) conditions and sequencing method. Therefore, it is likely that, after DNA damage repair, gRNA/Cas9-mediated HBV gene-editing by a single gRNA generates new HBV variants that are replication capable but less competent in supporting HBV cccDNA reproduction. Importantly, dual excision (i.e., by gRNA5 plus gRNA9) may lead to multiple DNA damages (which is supported by the DNA sequencing data) that are more difficult to repair. Thus, repeated treatment can increase the chance of cuttings in the uncut genes due to increased cell transfection rate with the dual gene-editing drugs, leading to a more pronounced (>95%) inhibition of HBV cccDNA formation. The most common outcome of viral cccDNA damage induced by CRISPR/Cas9 is the repair of DSBs by the NHEJ and/or HR pathways, which result in mutations and/or reduction in cccDNA if left unrepaired and thus can prevent the virus from escaping the gene-editing process and producing replicable new variants. Because HBV cccDNA methylation can affect the antiviral activity of CRISPR/Cas9-mediated gene excision, modulation of the DNA repair pathways to enhance CRISPR/Cas9 activity may facilitate the eradication of HBV cccDNA.

The integration of HBV DNA into the host genome is an obstacle for curing HBV infection and an important risk factor in hepatocarcinogenosis. While the integrated HBV DNA in cellular chromosomes is not a viral replicable template as HBV cccDNA (which can transcribe a full-length pgRNA to support viral replication), the integrated HBV DNA may contribute to HBV pathogenesis via the expression of large amounts of HBsAg and modulation of cellular gene expression and chromosomal stability. Many HBV integration events occur within fragile sites, such as TERT, FN1, MLL4, ROCK1, and SENP5, which are linked to oncogenesis and cancer development. A recent study demonstrated that the CRISPR/Cas9 RNP-based gene editing can modify the function of cccDNA and interrupt the replication of HBV DNA. In this study, it was found that synthetic gRNA/Cas9 RNPs can inhibit HBV DNA replication in multiple HBV cellular models with integrated HBV DNA, indicating that the CRISPR/Cas9 gene-editing approach can inactivate HBV gene expression from the integrated HBV DNA and potentially prevent chromosomal translocation and instability in the host cell genome.

The use of synthetic gRNA/Cas9 RNPs for gene-editing is an appealing approach because it can induce rapid DNA cleavage and has low off-target effects and low risk of oncogenic mutagenesis and can easily be produced for use in clinical applications. Nine gRNA target sites on HBV genomes were selected with the highest levels of conservation among HBV strains and the lowest homologous (off-target) on the host genome. The results showed that only gRNA5 (which targets P and S genomes) and gRNA9 (which targets P and X genomes) were the most specific and potent gRNAs in inactivating HBV replication. The regions targeted by these gRNAs contain the HBV polymerase, the S gene, and the viral enhancer and oncogenic X gene, which are all critical for HBV activity. Therefore, it is proposed that these genomic regions are optimal target sites for CRISPR/Cas9-mediated HBV gene-editing. These results warrant further investigation to test the efficacy of gRNA5 and gRNA9 RNPs in eradicating HBV infection. Theoretically, CRISPR/Cas9-mediated gene-editing/cleavage can only interfere with the translation of the target gene, however, it was noticed that the HBV DNA, cccDNA, and pgRNA levels were all suppressed following treatments with the gRNA RNPs. It is speculated that the lower levels of HBV RNA transcripts observed in this study are secondary to the inhibited HBV cccDNA, or that the introduced DNA mutations may lead to the instability of HBV RNA to a certain extent. Further studies are needed to illustrate the underlying mechanisms of these events.

In summary, this study provides proof-of-concept that synthetic gRNA/Cas9 RNPs can efficiently abolish HBV cccDNA replication and pgRNA/mRNA as well as HBsAg/HBeAg expression in infected cells in vitro.

### D. Example 4: Engineered Exosomes

The current antiviral treatment using nucleos(t)ide analogs (NAs) can only suppress de novo HBV replication but cannot eliminate HBV infection due to the persistence of HBV covalently closed circular (ccc) DNA, which sustains HBV replication. So far, there are no available drugs that can directly target HBV cccDNA. Therefore, novel curative therapeutics are urgently needed to eliminate HBV cccDNA from infected hepatocytes.

To address these unmet medical needs for people with chronic HBV infection (including Veterans who have a relative higher rate of HBV infection due to their higher rates of iv drug use and multiple sex partners), a series of synthetic gRNA/Cas9 gene-editing drugs targeting HBV cccDNA have been designed. A novel exosome-based delivery platform engineered to specifically deliver the HBV gene-editing drugs to human hepatocytes has also been developed. These engineered exosomes are designed in such a way that they carry an HBV pre-S1-derived peptide (PS1P) on the surface of exosomes so that they can more specifically deliver and intracellularly release the synthetic gRNA/Cas9 RNPs to HBV target cells (hepatocytes) expressing HBV receptor-NTCP. To this end, a plasmid expressing a fusion protein comprising three components: an extracellular PSIP, a CD9 transmembrane protein (CD9TP), and an intracellular red fluorescent protein (RFP) was constructed for tracking purpose. The plasmids expressing PSIP were transfected into human hepatocytes, as exosomes purified from the same origin of cell source are known to elicit minimal cytotoxicity or immunogenicity when administrated in vivo and thus are the most promising carrier for drug treatment. The exosomes were isolated from the supernatants of transfected cells using a differential ultracentrifugation protocol as we previously described. The purified exosomes were then packaged with the selected HBV-gRNA/Cas9 ribonucleoproteins (RNP) using electroporation method as a therapeutic drug ready to be used for HBV treatment. To further characterize this drug delivery system, the biophysical and biological features of these engineered exosomes carrying specific HBV gene therapy drug can be analyzed pre-clinically.

Targeted delivery of therapeutic drugs to specific cells are key to effective and less toxic treatment for human diseases. The goal of this study is to test an exosome delivery system packaged with synthetic gRNA/Cas9 ribonucleoprotein (RNP) and HBV pre-S1-derived peptide (PS1P) targeting HBV receptor NTCP (Na+ taurocholate co-transporting polypeptide) expressed on human hepatocytes for the purpose of HBV gene therapy. To this end, the engineered exosomes loaded with HBV gene-editing gRNA/Cas9 RNP drug and PS1P targeting hepatocytes expressing HBV receptor-NTCP can be used.

Despite successful control of viral replication in HBV patients using nucleos(t)ide analogs (NA), the current NA treatment cannot eliminate HBV infection due to integrated HBV DNA into host cell genome and that NAs have little effect on HBV cccDNA. Thus, any curative strategies should include means to eliminate the integrated HBV DNA and episomal cccDNA from infected cells without causing collateral cytotoxic effects.

Approximately 300 million people worldwide are chronically infected with HBV. US Veterans are at particularly high risk and disproportionately affected by HBV due to a variety of overlapping risk factors, including high rates of mental health disorders, substance abuse, and multiple sex partners. Thus, HBV infection remains a common problem among the Veteran populations and reinforces the need for integrated healthcare services along with novel therapeutic interventions. Therefore, this study is significant, timely, and can have a high impact on the healthcare needs of Military Service Members, Veterans, and the American public.

The goal of this translational study is to use primary human hepatocytes (PHHs) to produce exosomes packaged with gRNA/Cas9 RNPs and PSIP that has a high affinity for HBV receptor-NTCP.

It is hypothesized that the Exo-HBV-gRNA/Cas9 drugs can specifically target and efficiently abolish viral replication in HBV-infected hepatocytes and elicit minimal cytotoxic effects.

To test this hypothesis, the biophysical (a) and biological (b) features of the engineered exosome drugs targeting HBV-infected cells can be characterized.

Specific delivery and sustained release of the gRNA/Cas9 RNPs to HBV-infected hepatocytes are critical factors in developing an effective CRISPR-based HBV gene therapy. To characterize the biophysical (pharmaceutical) features of the exosome drugs, the exosome size, drug loading capacity, human hepatocyte targeting, cellular uptake, and drug release kinetics can be measured.

Specific inhibit viral replication and elicit minimum off-target side-effects are key parameters for a gene therapy drug. To characterize their biological features, the antiviral efficacy, off-target effect, and cytotoxicity of these exosome-delivered gene-editing drugs can be determined using multiple HBV-infected cellular models.

These pre-clinical studies can be performed to know the pharmaceutical and biological features of the engineered exosomes as a therapeutic drug for the treatment of HBV infection. The data generated from these experiments can help to optimize the exosome-based platform to advance this technology along the commercialization journey.

Comparing with existing viral and non-viral drug delivery methods, this engineered exosome platform has multiple advantages and benefit in drug delivery, including: 1) exosomes are highly biocompatible and non-cytotoxic to humans because they are purified from a compatible live cell source; 2) exosomes provide an immune-privileged protective drug delivery vehicle; 3) exosomes carry various membrane-anchored proteins that extend their half-life in blood/circulation by evading phagocytic clearance while conferring superior cellular uptake; 4) exosomes also can cross stringent biological barriers and can be engineered to specifically deliver encapsulated gRNA/Cas9 drugs to target cells.

### E. Example 5: Mice Study to Determine Potential Immune Response and Adverse Effects

While we have shown that synthetic gRNA/Cas9 RNPs do not exert detectable cytotoxic effects in HBV cellular models, questions remaining unanswered are whether these engineered exosomes carrying HBV gene-editing drugs can cause any adverse effects and/or immune responses in vivo.

6 NSG mice were treated with 100 μl PSIP exosome packaged with Cas9 (0.9 nmol). 6 NSG mice were treated with 100 μl PBS with Cas9 (0.9 nmol). The mice were subjected to tail vein injection every 3 days for 6 weeks.

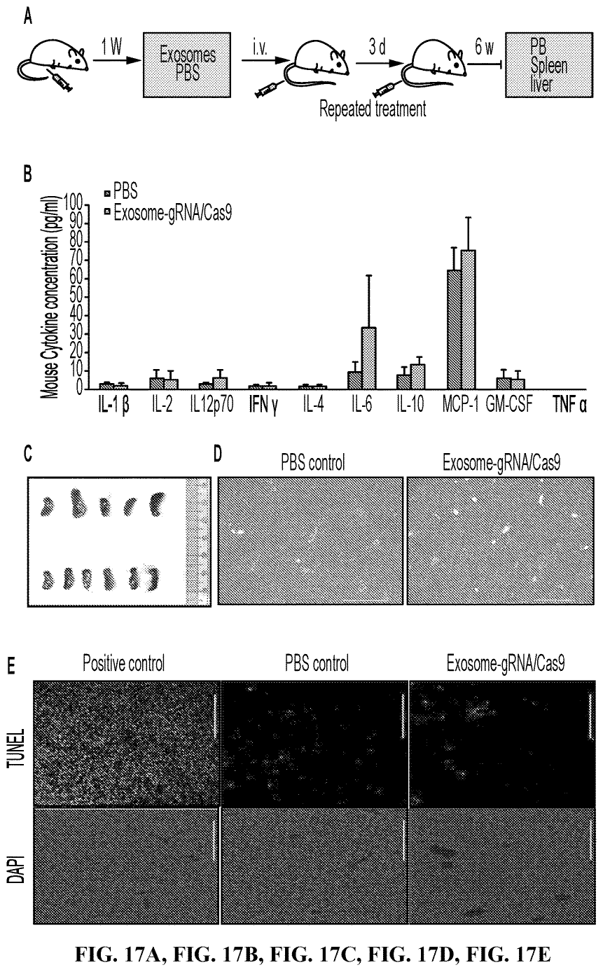

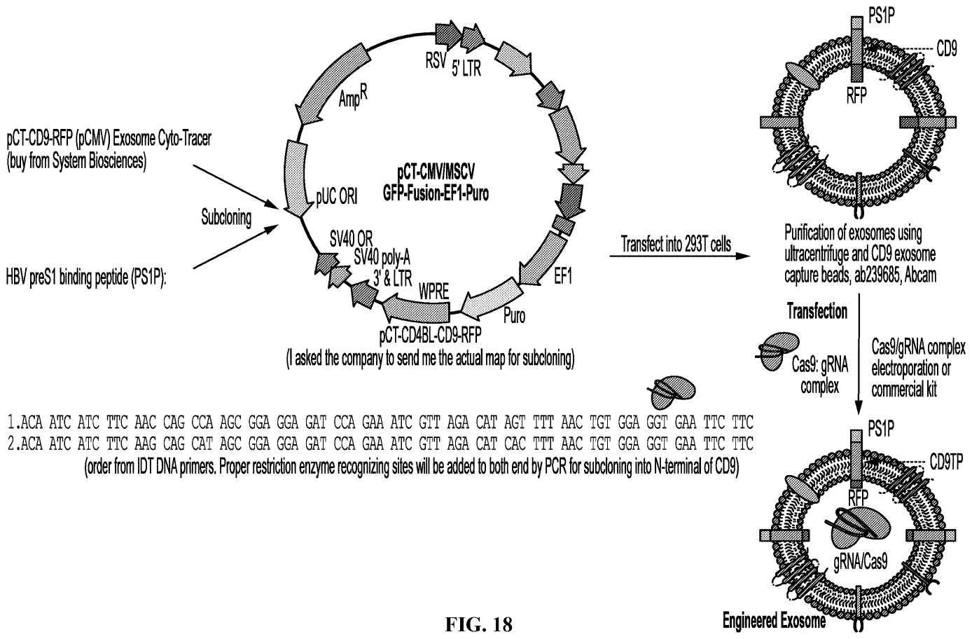

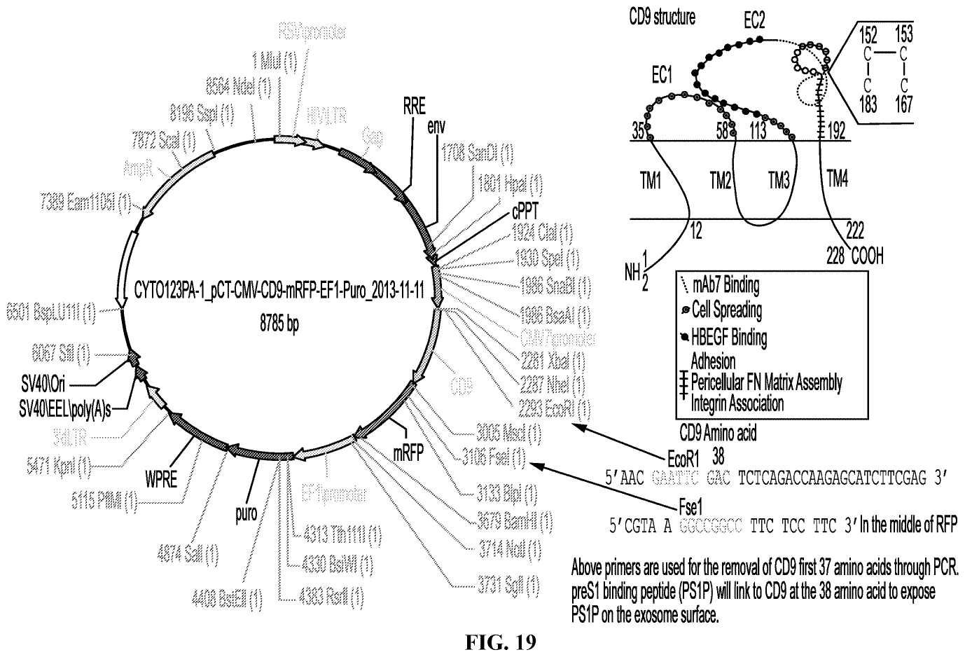

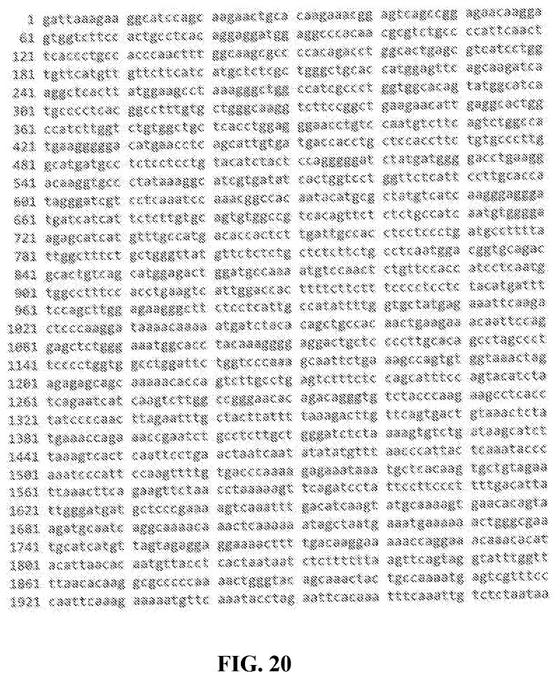

A Cas9 ELISA kit (MBS 169377) was used for measuring Cas9 protein in NSG mice serum. Cas 9 protein was detected in the exosome-Cas9 group, but not in the PBS-Cas9 group. Using anti-Cas9 ELISA kit, positive Cas9 antibody in the sera after 6 weeks treatment was not detected. The results of this study are shown in FIG. 17A-E.

NSG mice (6 mice per group) via tail vein with either 100 μl (0.9 nmol Cas9 RNPs) exosome drugs or PBS every 3 days for 6 weeks (FIG. 17A). Blood and major tissues were collected at end of the experiments. Using Cas9 protein and antibody ELISA kit (MBS 169377), we detected Cas9 protein, but not Cas9 antibody, in the blood of exosome-treated mice (data not shown). Cytokine array assay (Eve Technologies, Calgary, Canada) measuring the levels of inflammatory cytokines (including IL-1β, IL-2, IL-12p70, IFN-γ, IL-4, IL-6, IL-10, MCP-1, GM-CSF, and TNF-α, etc.) did not show any difference between exosome gRNA/Cas9-treated mice and PBS/Cas9-treated control mice (FIG. 17B). The speen weights were comparable between the two groups (FIG. 17C) and no inflammatory lymphocytes or monocytes were found infiltrated in the H.E. staining of livers from treated and control mice (FIG. 17D). No significant increases in apoptosis of hepatocytes by TUNEL staining of livers from exosme-gRNA/Cas9-treated and control mice (FIG. 17E). In Summary: NSG mice treated with exosomes carrying synthetic gRNA/Cas9 RNPs every 3 days for 6 weeks did not elicit 1) detectable antibodies against Cas9 protein, 2) increases in inflammatory cytokines, 3) inflammation of spleen and liver, and 4) apoptotic death of liver cells, compared to PBS-treated control mice.

Those skilled in the art can recognize, or be able to ascertain using no more than routine experimentation, many equivalents to the specific embodiments of the method and compositions described herein. Such equivalents are intended to be encompassed by the following claims.

### REFERENCES

1. Schweitzer A, Horn J, Mikolajczyk R T, Krause G, Ott J J. Estimations of worldwide prevalence of chronic hepatitis B virus infection: a systematic review of data published between 1965 and 2013. Lancet. 2015; 386 (10003): 1546-55. Epub 2015 Aug. 2. doi: 10.1016/S0140-6736 (15) 61412-X. PubMed PMID: 26231459.
2. Ott J J, Horn J, Krause G, Mikolajczyk R T. Time trends of chronic HBV infection over prior decades-A global analysis. J Hepatol. 2017; 66 (1): 48-54. Epub 2016 Sep. 7. doi: 10.1016/j.jhep.2016.08.013. PubMed PMID: 27592304.
3. Huang Y S, Sun H Y, Chang S Y, Chuang Y C, Cheng A, Huang S H, Huang Y C, Chen G J, Lin K Y, Su Y C, Liu W C, Hung C C. Long-term virological and serologic responses of chronic hepatitis B virus infection to tenofovir disoproxil fumarate-containing regimens in patients with HIV and hepatitis B coinfection. Hepatol Int. 2019; 13 (4): 431-9. Epub 2019 Jun. 10. doi: 10.1007/s12072-019-09953-4. PubMed PMID: 31177505.
4. Jiang T, Su B, Song T, Zhu Z, Xia W, Dai L, Wang W, Zhang T, Wu H. Immunological Efficacy of Tenofovir Disproxil Fumarate-Containing Regimens in Patients With HIV-HBV Coinfection: A Systematic Review and Meta-Analysis. Front Pharmacol. 2019; 10:1023. Epub 2019 Oct. 2. doi: 10.3389/fphar.2019.01023. PubMed PMID: 31572195; PMCID: PMC6752181.
5. Gish R G, Given B D, Lai C L, Locarnini S A, Lau J Y, Lewis D L, Schluep T. Chronic hepatitis B: Virology, natural history, current management and a glimpse at future opportunities. Antiviral Res. 2015; 121:47-58. Epub 2015 Jun. 21. doi: 10.1016/j.antiviral.2015.06.008. PubMed PMID: 26092643.
6. Koumbi L. Current and future antiviral drug therapies of hepatitis B chronic infection. World J Hepatol. 2015; 7 (8): 1030-40. Epub 2015 Jun. 9. doi: 10.4254/wjh.v7.i8.1030. PubMed PMID: 26052392; PMCID: PMC4450180.
7 Dusheiko G. Will we need novel combinations to cure HBV infection? Liver Int. 2020; 40 Suppl 1:35-42. Epub 2020 Feb. 23. doi: 10.1111/liv.14371. PubMed PMID: 32077595.
8. Nassal M. HBV cccDNA: viral persistence reservoir and key obstacle for a cure of chronic hepatitis B. Gut. 2015; 64 (12): 1972-84. Epub 2015 Jun. 7. doi: 10.1136/gutjnl-2015-309809. PubMed PMID: 26048673.
9 Guo J T, Guo H. Metabolism and function of hepatitis B virus cccDNA: Implications for the development of cccDNA-targeting antiviral therapeutics. Antiviral Res. 2015; 122:91-100. Epub 2015 Aug. 15. doi:10.1016/j.antiviral.2015.08.005. PubMed PMID: 26272257; PMCID: PMC4586118.
10. Zhu A, Liao X, Li S, Zhao H, Chen L, Xu M, Duan X. HBV cccDNA and Its Potential as a Therapeutic Target. J Clin Transl Hepatol. 2019; 7 (3): 258-62. Epub 2019 Oct. 15. doi: 10.14218/JCTH.2018.00054. PubMed PMID: 31608218; PMCID: PMC6783673.
11. Maepa M B, Jacobs R, van den Berg F, Arbuthnot P. Recent developments with advancing gene therapy to treat chronic infection with hepatitis B virus. Curr Opin HIV AIDS. 2020; 15 (3): 200-7. Epub 2020 Mar. 7. doi: 10.1097/COH.0000000000000623. PubMed PMID: 32141890.
12. Yang H C, Chen P J. The potential and challenges of CRISPR-Cas in eradication of hepatitis B virus covalently closed circular DNA. Virus Res. 2018; 244:304-10. Epub 2017 Jun. 20. doi: 10.1016/j.virusres.2017.06.010. PubMed PMID: 28627393.
13. Kostyusheva A P, Kostyushev D S, Brezgin S A, Zarifyan D N, Volchkova E V, Chulanov V P. [Small Molecular Inhibitors of DNA Double Strand Break Repair Pathways Increase the ANTI-HBV Activity of CRISPR/Cas9]. Mol Biol (Mosk). 2019; 53 (2): 311-23. Epub 2019 May 18. doi: 10.1134/S0026898419010075. PubMed PMID: 31099781.
14. Kostyushev D, Brezgin S, Kostyusheva A, Zarifyan D, Goptar I, Chulanov V. Orthologous CRISPR/Cas9 systems for specific and efficient degradation of covalently closed circular DNA of hepatitis B virus. Cell Mol Life Sci. 2019; 76 (9): 1779-94. Epub 2019 Jan. 24. doi: 10.1007/s00018-019-03021-8. PubMed PMID: 30673820.
15. Li H, Sheng C, Wang S, Yang L, Liang Y, Huang Y, Liu H, Li P, Yang C, Yang X, Jia L, Xie J, Wang L, Hao R, Du X, Xu D, Zhou J, Li M, Sun Y, Tong Y, Li Q, Qiu S, Song H. Removal of Integrated Hepatitis B Virus DNA Using CRISPR-Cas9. Front Cell Infect Microbiol. 2017; 7:91. Epub 2017 Apr. 7. doi: 10.3389/fcimb.2017.00091. PubMed PMID: 28382278; PMCID: PMC5360708.
16. Li H, Sheng C, Liu H, Wang S, Zhao J, Yang L, Jia L, Li P, Wang L, Xie J, Xu D, Sun Y, Qiu S, Song H. Inhibition of HBV Expression in HBV Transgenic Mice Using AAV-Delivered CRISPR-SaCas9. Front Immunol. 2018; 9:2080. Epub 2018 Sep. 27. doi: 10.3389/fimmu.2018.02080. PubMed PMID: 30254645; PMCID: PMC6141737.
17. Jiang C, Mei M, Li B, Zhu X, Zu W, Tian Y, Wang Q, Guo Y, Dong Y, Tan X. A non-viral CRISPR/Cas9 delivery system for therapeutically targeting HBV DNA and pcsk9 in vivo. Cell Res. 2017; 27 (3): 440-3. Epub 2017 Jan. 25. doi: 10.1038/cr.2017.16. PubMed PMID: 28117345; PMCID: PMC5339835.
18. Roth T L, Puig-Saus C, Yu R, Shifrut E, Carnevale J, Li P J, Hiatt J, Saco J, Krystofinski P, Li H, Tobin V, Nguyen D N, Lee M R, Putnam A L, Ferris A L, Chen J W, Schickel J N, Pellerin L, Carmody D, Alkorta-Aranburu G, Del Gaudio D, Matsumoto H, Morell M, Mao Y, Cho M, Quadros R M, Gurumurthy C B, Smith B, Haugwitz M, Hughes S H, Weissman J S, Schumann K, Esensten J H, May A P, Ashworth A, Kupfer G M, Greeley SAW, Bacchetta R, Meffre E, Roncarolo M G, Romberg N, Herold K C, Ribas A, Leonetti M D, Marson A. Reprogramming human T cell function and specificity with non-viral genome targeting. Nature. 2018; 559 (7714): 405-9. Epub 2018 Jul. 12. doi: 10.1038/s41586-018-0326-5. PubMed PMID: 29995861; PMCID: PMC6239417.
19. Wang M, Glass Z A, Xu Q. Non-viral delivery of genome-editing nucleases for gene therapy. Gene Ther. 2017; 24 (3): 144-50. Epub 2016 Nov. 1. doi: 10.1038/gt.2016.72. PubMed PMID: 27797355.
20. Zhang S, Shen J, Li D, Cheng Y. Strategies in the delivery of Cas9 ribonucleoprotein for CRISPR/Cas9 genome editing. Theranostics. 2021; 11 (2): 614-48. Epub 2021 Jan. 5. doi: 10.7150/thno.47007. PubMed PMID: 33391496; PMCID: PMC7738854.
21. Cai D, Mills C, Yu W, Yan R, Aldrich C E, Saputelli J R, Mason W S, Xu X, Guo J T, Block T_{M}, Cuconati A, Guo H. Identification of disubstituted sulfonamide compounds as specific inhibitors of hepatitis B virus covalently closed circular DNA formation. Antimicrob Agents Chemother. 2012; 56 (8): 4277-88. Epub 2012 May 31. doi: 10.1128/AAC.00473-12. PubMed PMID: 22644022; PMCID: PMC3421587.
22. Liu Y, Nie H, Mao R, Mitra B, Cai D, Yan R, Guo J T, Block T_{M}, Mechti N, Guo H. Interferon-inducible ribonuclease ISG20 inhibits hepatitis B virus replication through directly binding to the epsilon stem-loop structure of viral RNA. PLOS Pathog. 2017; 13 (4): e1006296. Epub 2017 Apr. 12. doi: 10.1371/journal.ppat. 1006296. PubMed PMID: 28399146; PMCID: PMC5388505.
23. Long Q, Yan R, Hu J, Cai D, Mitra B, Kim E S, Marchetti A, Zhang H, Wang S, Liu Y, Huang A, Guo H. The role of host DNA ligases in hepadnavirus covalently closed circular DNA formation. PLOS Pathog. 2017; 13 (12): e1006784. Epub 2017 Dec. 30. doi: 10.1371/journal.ppat. 1006784. PubMed PMID: 29287110; PMCID: PMC5747486.
24. Marchetti A L, Zhang H, Kim E S, Yu X, Jang S, Wang M, Guo H. Proteomic Analysis of Nuclear HBV rcDNA Associated Proteins Identifies U V-DDB as a Host Factor Involved in cccDNA Formation. J Virol. 2021: JVI0136021. Epub 2021 Oct. 28. doi: 10.1128/JVI.01360-21. PubMed PMID: 34705558.
25. Yan R, Zhang Y, Cai D, Liu Y, Cuconati A, Guo H. Spinoculation Enhances HBV Infection in NTCP-Reconstituted Hepatocytes. PLOS One. 2015; 10 (6): e0129889. Epub 2015 Jun. 13. doi: 10.1371/journal.pone.0129889. PubMed PMID: 26070202; PMCID: PMC4466484.
26. Marchetti M, Zermatten M G, Bertaggia Calderara D, Aliotta A, Alberio L. Heparin-Induced Thrombocytopenia: A Review of New Concepts in Pathogenesis, Diagnosis, and Management. J Clin Med. 2021; 10 (4). Epub 2021 Feb. 14. doi: 10.3390/jcm10040683. PubMed PMID: 33578859; PMCID: PMC7916628.
27. Croagh C M, Desmond P V, Bell S J. Genotypes and viral variants in chronic hepatitis B: A review of epidemiology and clinical relevance. World J Hepatol. 2015; 7 (3): 289-303. Epub 2015 Apr. 8. doi: 10.4254/wjh.v7.i3.289. PubMed PMID: 25848459; PMCID: PMC4381158.
28. Sells M A, Chen M L, Acs G. Production of hepatitis B virus particles in Hep G2 cells transfected with cloned hepatitis B virus DNA. Proc Natl Acad Sci USA. 1987;84 (4): 1005-9. Epub 1987 Feb. 1. doi: 10.1073/pnas.84.4.1005. PubMed PMID: 3029758; PMCID: PMC304350.
29. Yan H, Zhong G, Xu G, He W, Jing Z, Gao Z, Huang Y, Qi Y, Peng B, Wang H, Fu L, Song M, Chen P, Gao W, Ren B, Sun Y, Cai T, Feng X, Sui J, Li W. Sodium taurocholate cotransporting polypeptide is a functional receptor for human hepatitis B and D virus. Elife. 2012; 1: e00049. Epub 2012 Nov. 15. doi: 10.7554/eLife.00049. PubMed PMID: 23150796; PMCID: PMC3485615.
30. Tong S, Li J. Identification of NTCP as an HBV receptor: the beginning of the end or the end of the beginning? Gastroenterology. 2014; 146 (4): 902-5. Epub 2014 Mar. 1. doi: 10.1053/j.gastro.2014.02.024. PubMed PMID: 24576732; PMCID: PMC6342000.
31. Bibikova M, Carroll D, Segal D J, Trautman J K, Smith J, Kim Y G, Chandrasegaran S. Stimulation of homologous recombination through targeted cleavage by chimeric nucleases. Mol Cell Biol. 2001; 21 (1): 289-97. Epub 2000 Dec. 13. doi: 10.1128/MCB.21.1.289-297.2001. PubMed PMID: 11113203; PMCID: PMC88802.
32. Guo H, Jiang D, Zhou T, Cuconati A, Block T_{M}, Guo J T. Characterization of the intracellular deproteinized relaxed circular DNA of hepatitis B virus: an intermediate of covalently closed circular DNA formation. J Virol. 2007; 81 (22): 12472-84. Epub 2007 Sep. 7. doi: 10.1128/JVI.01123-07. PubMed PMID: 17804499; PMCID: PMC2169032.
33. Cai D, Wang X, Yan R, Mao R, Liu Y, Ji C, Cuconati A, Guo H. Establishment of an inducible HBV stable cell line that expresses cccDNA-dependent epitope-tagged HBeAg for screening of cccDNA modulators. Antiviral Res. 2016; 132:26-37. Epub 2016 May 18. doi: 10.1016/j.antiviral.2016.05.005. PubMed PMID: 27185623; PMCID: PMC5060949.
34. Seeger C, Sohn J A. Targeting Hepatitis B Virus With CRISPR/Cas9. Mol Ther Nucleic Acids. 2014; 3: e216. Epub 2014 Dec. 17. doi: 10.1038/mtna.2014.68. PubMed PMID: 25514649; PMCID: PMC4272409.
35. Kennedy E M, Bassit L C, Mueller H, Kornepati AVR, Bogerd H P, Nie T, Chatterjee P, Javanbakht H, Schinazi R F, Cullen B R. Suppression of hepatitis B virus DNA accumulation in chronically infected cells using a bacterial CRISPR/Cas RNA-guided DNA endonuclease. Virology. 2015; 476:196-205. Epub 2015 Jan. 2. doi: 10.1016/j.virol.2014.12.001. PubMed PMID: 25553515; PMCID: PMC4323668.
36. Martinez. M G, Combe E, Inchauspe A, Mangeot P E, Delberghe E, Chapus F, Neveu G, Alam A, Carter K, Testoni B, Zoulim F. CRISPR-Cas9 Targeting of Hepatitis B Virus Covalently Closed Circular DNA Generates Transcriptionally Active Episomal Variants. mBio. 2022; 13 (2): e0288821. Epub 2022 Apr. 8. doi: 10.1128/mbio.02888-21. PubMed PMID: 35389262; PMCID: PMC9040760.
37. Zhen S, Hua L, Liu Y H, Gao L C, Fu J, Wan D Y, Dong L H, Song H F, Gao X. Harnessing the clustered regularly interspaced short palindromic repeat (CRISPR)/CRISPR-associated Cas9 system to disrupt the hepatitis B virus. Gene Ther. 2015; 22 (5): 404-12. Epub 2015 Feb. 6. doi: 10.1038/gt.2015.2. PubMed PMID: 25652100.
38. Seeger C, Sohn J A. Complete Spectrum of CRISPR/Cas9-induced Mutations on HBV cccDNA. Mol Ther. 2016; 24 (7): 1258-66. Epub 2016 May 21. doi: 10.1038/mt.2016.94. PubMed PMID: 27203444; PMCID: PMC5088770.
39. Dong C, Qu L, Wang H, Wei L, Dong Y, Xiong S. Targeting hepatitis B virus cccDNA by CRISPR/Cas9 nuclease efficiently inhibits viral replication. Antiviral Res. 2015; 118:110-7. Epub 2015 Apr. 7. doi: 10.1016/j.antiviral.2015.03.015. PubMed PMID: 25843425.
40. Xu C, Zhou W, Wang Y, Qiao L. Hepatitis B virus-induced hepatocellular carcinoma. Cancer Lett. 2014; 345 (2): 216-22. Epub 2013 Aug. 29. doi: 10.1016/j.canlet.2013.08.035. PubMed PMID: 23981576.
41. Tarocchi M, Polvani S, Marroncini G, Galli A. Molecular mechanism of hepatitis B virus-induced hepatocarcinogenesis. World J Gastroenterol. 2014; 20 (33): 11630-40. Epub 2014 Sep. 11. doi: 10.3748/wjg.v20.i33.11630. PubMed PMID: 25206269; PMCID: PMC4155355.
42. Sung W K, Zheng H, Li S, Chen R, Liu X, Li Y, Lee N P, Lee W H, Ariyaratne P N, Tennakoon C, Mulawadi F H, Wong K F, Liu A M, Poon R T, Fan S T, Chan K L, Gong Z, Hu Y, Lin Z, Wang G, Zhang Q, Barber T D, Chou W C, Aggarwal A, Hao K, Zhou W, Zhang C, Hardwick J, Buser C, Xu J, Kan Z, Dai H, Mao M, Reinhard C, Wang J, Luk J M. Genome-wide survey of recurrent HBV integration in hepatocellular carcinoma. Nat Genet. 2012; 44 (7): 765-9. Epub 2012 May 29. doi: 10.1038/ng.2295. PubMed PMID: 22634754.
43. Jiang Z, Jhunjhunwala S, Liu J, Haverty P M, Kennemer M I, Guan Y, Lee W, Carnevali P, Stinson J, Johnson S, Diao J, Yeung S, Jubb A, Ye W, Wu T D, Kapadia S B, de Sauvage F J, Gentleman R C, Stern H M, Seshagiri S, Pant K P, Modrusan Z, Ballinger D G, Zhang Z. The effects of hepatitis B virus integration into the genomes of hepatocellular carcinoma patients. Genome Res. 2012; 22 (4): 593-601. Epub 2012 Jan. 24. doi: 10.1101/gr. 133926.111. PubMed PMID: 22267523; PMCID: PMC3317142.
44. Hai H, Tamori A, Kawada N. Role of hepatitis B virus DNA integration in human hepatocarcinogenesis. World J Gastroenterol. 2014; 20 (20): 6236-43. Epub 2014 May 31. doi: 10.3748/wjg.v20.i20.6236. PubMed PMID: 24876744; PMCID: PMC4033461.
45. Feitelson M A, Lee J. Hepatitis B virus integration, fragile sites, and hepatocarcinogenesis. Cancer Lett. 2007; 252 (2): 157-70. Epub 2006 Dec. 26. doi: 10.1016/j.canlet.2006.11.010. PubMed PMID: 17188425.
46. Zoulim F, Locarnini S. Hepatitis B virus resistance to nucleos(t)ide analogues. Gastroenterology. 2009; 137 (5): 1593-608 el-2. Epub 2009 Sep. 10. doi: 10.1053/j.gastro.2009.08.063. PubMed PMID: 19737565.
47. O'Geen H, Henry I M, Bhakta M S, Meckler J F, Segal D J. A genome-wide analysis of Cas9 binding specificity using ChIP-seq and targeted sequence capture. Nucleic Acids Res. 2015; 43 (6): 3389-404. Epub 2015 Feb. 26. doi: 10.1093/nar/gkv137. PubMed PMID: 25712100; PMCID: PMC4381059.
48. Morgens D W, Wainberg M, Boyle E A, Ursu O, Araya C L, Tsui C K, Haney M S, Hess G T, Han K, Jeng E E, Li A, Snyder M P, Greenleaf W J, Kundaje A, Bassik M C. Genome-scale measurement of off-target activity using Cas9 toxicity in high-throughput screens. Nat Commun. 2017; 8:15178. Epub 2017 May 6. doi: 10.1038/ncomms15178. PubMed PMID: 28474669; PMCID: PMC5424143.
49. Cameron P, Fuller C K, Donohoue P D, Jones B N, Thompson M S, Carter M M, Gradia S, Vidal B, Garner E, Slorach E M, Lau E, Banh L M, Lied A M, Edwards L S, Settle A H, Capurso D, Llaca V, Deschamps S, Cigan M, Young J K, May A P. Mapping the genomic landscape of CRISPR-Cas9 cleavage. Nat Methods. 2017; 14 (6): 600-6. Epub 2017 May 2. doi: 10.1038/nmeth.4284. PubMed PMID: 28459459.
50. Koo T, Lee J, Kim J S. Measuring and Reducing Off-Target Activities of Programmable Nucleases Including CRISPR-Cas9. Mol Cells. 2015; 38 (6): 475-81. Epub 2015 May 20. doi: 10.14348/molcells.2015.0103. PubMed PMID: 25985872; PMCID: PMC4469905.
51. Wilbie D, Walther J, Mastrobattista E. Delivery Aspects of CRISPR/Cas for in Vivo Genome Editing. Acc Chem Res. 2019; 52 (6): 1555-64. Epub 2019 May 18. doi: 10.1021/acs.accounts.9b00106. PubMed PMID: 31099553; PMCID: PMC6584901.
52. Yin H, Kauffman K J, Anderson D G. Delivery technologies for genome editing. Nat Rev Drug Discov. 2017; 16 (6): 387-99. Epub 2017 Mar. 25. doi: 10.1038/nrd.2016.280. PubMed PMID: 28337020.
53. You L, Tong R, Li M, Liu Y, Xue J, Lu Y. Advancements and Obstacles of CRISPR-Cas9 Technology in Translational Research. Mol Ther Methods Clin Dev. 2019; 13:359-70. Epub 2019 Apr. 17. doi: 10.1016/j.omtm.2019.02.008. PubMed PMID: 30989086; PMCID: PMC6447755.
54. Louis Jeune V, Joergensen J A, Hajjar R J, Weber T. Pre-existing anti-adeno-associated virus antibodies as a challenge in AAV gene therapy. Hum Gene Ther Methods. 2013; 24 (2): 59-67. Epub 2013 Feb. 28. doi: 10.1089/hgtb.2012.243. PubMed PMID: 23442094; PMCID: PMC3732124.
55. Rapti K, Louis-Jeune V, Kohlbrenner E, Ishikawa K, Ladage D, Zolotukhin S, Hajjar R J, Weber T. Neutralizing antibodies against AAV serotypes 1, 2, 6, and 9 in sera of commonly used animal models. Mol Ther. 2012; 20 (1): 73-83. Epub 2011 Sep. 15. doi: 10.1038/mt.2011.177. PubMed PMID: 21915102; PMCID: PMC3255603.
56. Nguyen D N, Roth T L, Li P J, Chen P A, Apathy R, Mamedov M R, Vo L T, Tobin V R, Goodman D, Shifrut E, Bluestone J A, Puck J M, Szoka F C, Marson A. Polymer-stabilized Cas9 nanoparticles and modified repair templates increase genome editing efficiency. Nat Biotechnol. 2020; 38 (1): 44-9. Epub 2019 Dec. 11. doi: 10.1038/s41587-019-0325-6. PubMed PMID: 31819258; PMCID: PMC6954310.
57. Wessel E M, Tomich J M, Todd R B. Biodegradable Drug-Delivery Peptide Nanocapsules. ACS Omega. 2019; 4 (22): 20059-63. Epub 2019 Dec. 4. doi: 10.1021/acsomega.9b03245. PubMed PMID: 31788640; PMCID: PMC6882115 stockholder in the firm Phoreus Biotechnology that has licensed the I P developed by Professor Tomich at Kansas State University. All of the research for this project was carried out in a non-affiliated laboratory. EMW and RBT have no conflicts to declare.
58. Chen G, Abdeen A A, Wang Y, Shahi P K, Robertson S, Xie R, Suzuki M, Pattnaik B R, Saha K, Gong S. A biodegradable nanocapsule delivers a Cas9 ribonucleoprotein complex for in vivo genome editing. Nat Nanotechnol. 2019; 14 (10): 974-80. Epub 2019 Sep. 11. doi: 10.1038/s41565-019-0539-2. PubMed PMID: 31501532; PMCID: PMC6778035.

We claim:

1. An exosome comprising a Na+ taurocholate co-transporting polypeptide (NTCP) binding motif.

2. The exosome of claim 1, wherein the exosome is a hepatocyte-derived exosome, mesenchymal stem cell (MSC) derived exosome, 293T cell derived exosome, or liver stellate cell-derived exosomes.

3. The exosome of claim 1, wherein the NTCP binding motif is expressed on the surface of the exosome.

4. The exosome of claim 1, wherein the NTCP binding motif is a fragment of an HBV preS1 peptide (PS1P).

5. The exosome of any one of claims 1-4, wherein the NTCP binding motif is fused to a transmembrane protein.

6. The exosome of claim 5, wherein the transmembrane protein is CD9, CD63 or CD81.

7. The exosome of claim 6, wherein the transmembrane protein is CD9.

8. The exosome of claim 7, wherein the CD9 is truncated.

9. The exosome of any of the preceding claims, further comprising a therapeutic agent.

10. The exosome of claim 8, wherein the therapeutic agent is a gRNA/Cas ribonucleoprotein complex comprising a gRNA and a Cas protein.

11. The exosome of claim 10, wherein the gRNA targets a region of the HBV genome comprising the polymerase gene, capsid gene, surface protein genes, or X genes.

12. The exosome of claim 10 or 11, wherein the gRNA comprises the sequence of one or more of the gRNAs in Table 1-3.

13. The exosome of any of claims 10-12, wherein the Cas protein of the gRNA/Cas ribonucleoprotein complex is Cas9, Cas12, or Cas13.

14. The exosome of any of the preceding claims, wherein the exosomes have a diameter of about 30-160 nm.

15. A method of making an engineered exosome comprising:
a) transfecting a plasmid into a cell, wherein the plasmid comprises a nucleic acid sequence capable of encoding a fusion protein, wherein the fusion protein comprises a transmembrane protein and a target-specific binding motif;
b) optionally, screening for cells producing exosomes expressing the fusion protein;
c) culturing the cells to allow production of exosomes expressing the fusion protein;
d) obtaining an exosome-containing supernatant; and
e) optionally, isolating the engineered exosomes from the exosome-containing supernatant.

16. The method of claim 15, wherein the cell is a hepatocyte or mesenchymal stem cell (MSC).

17. The method of claim 15, wherein the target-specific binding motif is a NTCP binding motif.

18. The method of claim 17, wherein the NTCP binding motif is HBV PreS1-derived peptide (PSIP) or a fragment thereof.

19. The method of any one of claims 15-18, wherein the transmembrane protein is CD9, CD63 or CD81.

20. The method of claim 19, wherein the transmembrane protein is truncated CD9.

21. The method of any one of claims 15-20, wherein isolating comprises ultra-centrifuging the exosome-containing supernatant to isolate the engineered exosomes.

22. The method of any one of claims 15-21, wherein isolating comprises purifying the engineered exosomes from exosome-containing supernatant using column chromatography.

23. The method of any one of claims 15-22, wherein the fusion protein further comprises a marker.

24. The method of claim 23, wherein the marker is a fluorescent protein.

25. The method of claim 24, wherein the fluorescent protein is red fluorescent protein (RFP) or green fluorescent protein (GFP).

26. The method of any of claims 15-23, wherein the exosomes have a diameter of about 30-160 nm.

27. The method of any one of claims 15-26, further comprising loading the exosomes with a therapeutic agent.

28. The method of claim 27, wherein loading comprises electroporation.

29. The method of any one of claims 27-28, wherein the therapeutic agent is a HBV specific gRNA/Cas ribonucleoprotein complex comprising a HBV specific gRNA and a Cas protein.

30. The method of claim 29, wherein the gRNA targets a region of the HBV genome comprising the polymerase gene, capsid gene, surface protein genes, or X genes.

31. The method of claim 29 or 30, wherein the gRNA comprises one or more of the sequences of Table 1-3.

32. The method of any one of claims 29-31, wherein the Cas protein of the gRNA/Cas ribonucleoprotein complex is Cas9, Cas12, or Cas13.

33. The method of any one of claims 27-28, wherein the therapeutic agent is lamivudine, telbivudine, entecavir, adefovir dipivoxil or tenofovir disoproxil fumarate.

34. A method of treating a subject infected with hepatitis B virus (HBV) comprising administering to the subject a therapeutically effective amount of exosomes, wherein the exosomes comprise a NTCP binding motif and a therapeutic agent, thereby treating the HBV infection in the subject.

35. The method of claim 34, wherein the exosomes are hepatocyte derived exosomes.

36. The method of any one of claims 34-35, wherein the NTCP binding motif is HBV PS1P.

37. The method of any one of claims 34-36, wherein the therapeutic agent is inside of the exosomes.

38. The method of any one of claims 34-37, wherein the therapeutic agent is an HBV specific gRNA/Cas ribonucleoprotein.

39. The method of claim 38, wherein the HBV specific gRNA targets a region of the HBV genome comprising the polymerase gene, capsid gene, surface protein genes, or X genes.

40. The method of claim 38 or 39, wherein the gRNA comprises one or more of the sequences of Table 1, Table 2, or Table 3.

41. The method of any one of claims 38-40, wherein the Cas protein of the gRNA/Cas ribonucleoprotein complex is Cas9, Cas12, or Cas13.

42. The method of any one of claims 34-37, wherein the therapeutic agent is lamivudine, telbivudine, entecavir, adefovir dipivoxil or tenofovir disoproxil fumarate.

43. The method of any one of claims 34-42, wherein the NTCP binding motif is fused to a transmembrane domain.

44. The method of claim 43, wherein the transmembrane domain is CD9.

45. The method of claim 44, wherein the CD9 is truncated.

46. The method of any one of claims 34-45, wherein a therapeutically effective amount of exosomes comprises between 1×10⁶ particles and 1×10⁸ particles.

47. The method of any one of claims 34-46, wherein administering is intravenous administration.

48. The method of any one of claims 36-39, 41-47, wherein the gRNA/Cas ribonucleoprotein cleaves HBV genomic DNA and/or RNA in an HBV infected cell of the subject.

49. The method of any one of claims 34-37, 42-48, wherein the therapeutic agent inhibits DNA polymerase.

50. A method of inactivating HBV cccDNA in HBV infected cells comprising administering to the HBV infected cells a therapeutically effective amount of exosomes, wherein the exosomes comprise an extracellular NTCP binding motif and a therapeutic agent, thereby inactivating HBV cccDNA and/or iDNA in the HBV infected cells.

51. The method of claim 50, wherein the HBV infected cells are in vitro.

52. The method of claim 50, wherein the HBV infected cells are in a subject.

53. The method of any one of claims 50-52, wherein the therapeutic agent is inside of the exosomes.

54. The method of any one of claims 50-53, wherein the therapeutic agent is an HBV specific gRNA/Cas ribonucleoprotein.

55. The method of claim 54, wherein the HBV specific gRNA targets a region of the HBV genome comprising the polymerase gene, capsid gene, surface protein genes, or X genes.

56. The method of claim 54 or 55, wherein the gRNA comprises one or more of the sequences of Table 1, Table 2, or Table 3.

57. The method of any one of claims 54-56, wherein the Cas protein of the gRNA/Cas ribonucleoprotein complex is Cas9, Cas12, or Cas13.

58. The method of any one of claims 50-52, wherein the therapeutic agent is is lamivudine, telbivudine, entecavir, adefovir dipivoxil or tenofovir disoproxil fumarate.

59. The method of any one of claims 50-58, wherein the NTCP binding motif is fused to a transmembrane domain.

60. The method of claim 59, wherein the transmembrane domain is CD9.

61. The method of claim 60, wherein the CD9 is truncated.

62. The method of any one of claims 50-61, wherein a therapeutically effective amount of exosomes comprises between 1×10⁶ particles and 1×10⁸ particles.

63. A method of delivering a therapeutic agent to a HBV target cells comprising administering to the HBV infected cells a therapeutically effective amount of exosomes, wherein the exosomes comprise a NTCP binding motif and a therapeutic agent.

64. A method of reducing HBV production from cells infected with HBV comprising administering to the HBV infected cells a therapeutically effective amount of exosomes, wherein the exosomes comprise a NTCP binding motif and a therapeutic agent, thereby reducing HBV production in the cells infected with HBV.

65. A method of cleaving a HBV genome in a cell infected with HBV comprising administering to the HBV infected cells a therapeutically effective amount of exosomes, wherein the exosomes comprise a NTCP binding motif and a therapeutic agent, wherein the therapeutic agent is a HBV specific gRNA/Cas ribonucleoprotein complex comprising a HBV specific gRNA and a Cas protein, thereby cleaving the HBV genome in the cell infected with HBV.

66. A method of inhibiting HBV replication in HBV infected cells comprising administering to the HBV infected cells a therapeutically effective amount of exosomes, wherein the exosomes comprise a NTCP binding motif and a therapeutic agent, thereby inhibiting HBV replication in the cell.

67. The method of any one of claims 63-66, wherein the HBV infected cells are in vitro.

68. The method of any one of claims 63-66, wherein the HBV infected cells are in a subject.

69. The method of any one of claims 63-68, wherein the exosomes are hepatocyte derived exosomes.

70. The method of any one of claims 63-69, wherein the therapeutic agent is inside of the exosomes.

71. The method of any one of claims 63-70, wherein the therapeutic agent is an HBV specific gRNA/Cas ribonucleoprotein.

72. The method of claim 71, wherein the HBV specific gRNA targets a region of the HBV genome comprising the polymerase gene, capsid gene, surface protein genes, or X genes.

73. The method of claim 71 or 72, wherein the gRNA comprises one or more of the sequences of Table 1, Table 2, or Table 3.

74. The method of any one of claims 70-73, wherein the Cas protein of the gRNA/Cas ribonucleoprotein complex is Cas9, Cas 12, or Cas13.

75. The method of any one of claims 63-70, wherein the therapeutic agent is lamivudine, telbivudine, entecavir, adefovir dipivoxil or tenofovir disoproxil fumarate.

76. The method of any one of claims 63-75, wherein the NTCP binding motif is fused to a transmembrane domain.

77. The method of claim 76, wherein the transmembrane domain is CD9, CD63 or CD81.

78. The method of claim 77, wherein the wherein the transmembrane protein is CD9.

79. The method of claim 78, wherein the CD9 is truncated.

80. The method of any one of claims 63-79, wherein a therapeutically effective amount of exosomes comprises between 1×10⁶ particles and 1×10⁸ particles.

81. A gRNA comprising any one of the sequences of Table 1, Table 2, or Table 3.

82. The gRNA of claim 81, wherein the gRNA is specific for a HBV polymerase (P), surface(S), or a HBV viral enhancer or oncogenic (X) genes.

83. The gRNA of claim 81-82, wherein the gRNA is based on the ayw strain of HBV (Genbank accession number: N C_003977.2).

84. A gRNA/Cas ribonucleoprotein complex comprising a gRNA and a Cas protein, wherein the gRNA comprises one or more of the sequences of Table 1, Table 2, or Table 3.

85. The gRNA/Cas ribonucleoprotein complex of claim 84, wherein the Cas protein is Cas9, Cas12, or Cas13.

86. The gRNA/Cas ribonucleoprotein complex of claim 84-85, wherein the gRNA is specific for targeting HBV polymerase (P), surface(S), and viral enhancer and oncogenic (X) genes.

87. The gRNA/Cas ribonucleoprotein complex of any one of claims 84-86, wherein the gRNA is based on the ayw strain of HBV (Genbank accession number: N C_003977.2)


SEQUENCE LISTING (67 sequences)

SEQ ID NO: 1 - protein, 46 aa, synthetic construct
GTNLSVPNPLGFFPDHQLDPAFGANSNNPDWDFNPNKDHWPEANQV

SEQ ID NO: 2 - DNA, 968 bp, synthetic construct
  source 1..968: mol_type = other DNA
ttctctcattttctagggggaactaccgtgtgtcttggccaaaattcgcagtccccaacctccaatcactcaccaacctcctgtcctccaacttgtcctggttatcactggatgtgtctgcggcgttttatcatcttcctcttcatcctgctgctatgcctcatcttcttgttggttcttctggactatcaaggtatgttgcccgtttgtcctcgaattccttgatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcnnntttcactttctcgccaacttacaaggcctttctgtgtaaacaatacctgaacctttaccccgttgcccggcaacggccaggtctgtgccaagtgtttgctgacgcaaccccnctggctggggcttggncatgggcntcagcgcgtg

SEQ ID NO: 3 - DNA, 968 bp, synthetic construct
  source 1..968: mol_type = other DNA
ttctctcattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcnnntttcactttctcgccaacttacaaggcctttctgtgtaaacaatacctgaacctttaccccgttgcccggcaacggccaggtctgtgccaagtgnttgctgacgcacccccactggctggggcttngncatgggnntcagcncgtg

SEQ ID NO: 4 - DNA, 936 bp, synthetic construct
  source 1..936: mol_type = other DNA
ggggccaagtctgtacagcatcttgagtccctttttaccgctgttaccaattttctttgtctttgggtatacatttaaacgtaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcntgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggttgacagggactctctcgtccccttatccgtttgccgttccgacggaccacggggcgcacctctctttacgcggactccccgtctgtgccttctcataggccggaccgtgtgcactttgattcacctctgcacgtcgcatggagaccaccgtgaacgcccaccgaatgttgcccaaggtcttacataagaggactcttggactctctncaatgtcaacgancgac

SEQ ID NO: 5 - DNA, 935 bp, synthetic construct
  source 1..935: mol_type = other DNA
ggggccaagtctgtacagcatntgagtcccttttaccgctgttaccaattttctttgtctttgggtatacatttaaacccnaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcntgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgancgac

SEQ ID NO: 6 - DNA, 78 bp, synthetic construct
  source 1..78: mol_type = other DNA
acaatcatcttcaaccagccaagcggaggagatccagaaatcgttagacatagttttaactgtggaggtgaattcttc

SEQ ID NO: 7 - DNA, 78 bp, synthetic construct
  source 1..78: mol_type = other DNA
acaatcatcttcaagcagcatagcggaggagatccagaaatcgttagacatcactttaactgtggaggtgaattcttc

SEQ ID NO: 8 - DNA, 36 bp, synthetic construct
  source 1..36: mol_type = other DNA
aacgaattcgactctcagaccaagagcatcttcgag

SEQ ID NO: 9 - DNA, 22 bp, synthetic construct
  source 1..22: mol_type = other DNA
cgtaaggccggccttctccttc

SEQ ID NO: 10 - DNA, 1980 bp, synthetic construct
  source 1..1980: mol_type = other DNA
gattaaagaaggcatccagcaagaactgcacaagaaacggagtcagccggagaacaaggagtggtcttccactgcctcacaggaggatggaggcccacaacgcgtctgccccattcaacttcaccctgccacccaactttggcaagcgccccacagacctggcactgagcgtcatcctggtgttcatgttgttcttcatcatgctctcgctgggctgcaccatggagttcagcaagatcaaggctcacttatggaagcctaaagggctggccatcgccctggtggcacagtatggcatcatgcccctcacggcctttgtgctgggcaaggtcttccggctgaagaacattgaggcactggccatcttggtctgtggctgctcacctggagggaacctgtccaatgtcttcagtctggccatgaagggggacatgaacctcagcattgtgatgaccacctgctccaccttctgtgcccttggcatgatgcctctcctcctgtacatctactccagggggatctatgatggggacctgaaggacaaggtgccctataaaggcatcgtgatatcactggtcctggttctcattccttgcaccatagggatcgtcctcaaatccaaacggccacaatacatgcgctatgtcatcaagggagggatgatcatcattctcttgtgcagtgtggccgtcacagttctctctgccatcaatgtggggaagagcatcatgtttgccatgacaccactcttgattgccacctcctccctgatgccttttattggctttctgctgggttatgttctctctgctctcttctgcctcaatggacggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaaggataaaacaaaaatgatctacacagctgccacaactgaagaaacaattccaggagctctgggaaatggcacctacaaaggggaggactgctccccttgcacagcctagcccttcccctggtggcctggattctggtcccaaagcaattctgaaagccagtgtggtaaactagagagagcagcaaaaacaccagtcttgcctgagtctttctccagcatttccagtacatctatcagaatcatcaagtcttggccgggaacacagacagggtgtctacccaagaagcctcacctatccccaacttagaatttgctacttattttaaagacttgttcagtgactgtaaactctatgaaaccagaaaccgaatctgcctcttgctgggatctctaaaagtgtctgataagcatcttaaagtcactcaattcctgaactaatcaatatatatgtttaacccattactcaaatacccaaatcccattccaagttttgtgacccaaaagagaaataaatgctcacaagtgctgtagaattaaacttcagaagttctaacctaaaaagttcagatcctattccttcccttttgacattattgggatgatgctcccgaaaagtcaaatttgacatcaagtatgcaaaagtgaacacagtaagatgcaatcaggcaaaacaaactcaaaaaatagctaatgaaatgaaaaaactgggcgaatgcatcatgttagtagaggaggaaaacttttgacaaggaaaaaccaggaaacaaacacatacattaacacaatgttacctcactaataatctcttttttaagttcagtaggtatttggttttaacacaaggcgcccccaaaactgggtacagcaaactactgccaaaatgagtcgtttcccaattcaaagaaaaatgttcaaatacctagaattcacaaatttcaaattgtctctaataa

SEQ ID NO: 11 - protein, 379 aa, synthetic construct
MEAHNLSAPLNFTLPPNFGKRPTDQALSVILVVMLLIMMLSLGCTMEFSKIKAHFWKPKGLAIALLAQYGIMPLTAFALGKVFPLNNIEALAILVCGCSPGGNLSNVFSLAMKGDMNLSIVMTTCSTFFALGMMPLLLYIYSKGIYDGDLKDKVPYVGIVISLILVLIPCTIGIFLKSKRPQYVPYVTKAGMIIILLLSVAITVLSVINVGKSIMFVMTPHLLATSSLMPFIGFLLGYILSTLFRLNAQCSRTVSMETGCQNVQLCSTILNVTFRPEVIGPLFFFPLLYMIFQLGEGLLLIAIYRCYEKIKTSKDKTKVIYTAAKTEETIPGTLGNSTHKCEEYSPYTVENSTHKCEEYSPSTVGNGTYKGEECSPGTA

SEQ ID NO: 12 - DNA, 366 bp, synthetic construct
  source 1..366: mol_type = other DNA
atttacacaccctgtggaaggcgggcatcttatataagagagaaactacacgtagcgcctcattttgtgggtcaccatattcttgggaacaagatctacagcatggggcagaatcattctgtcaccaatcctttgggattgttccccgatcatcagttggaccctctgttcagagccaactcaaacaatccagattgggacttcaaccccaacaaagacacctggccagaagccaccaaggtaggagtgggggcattcgggccagggttcacccctccacatggcggccttttggggtggagcccccaggctcagggcatattaacaacattgccagcggcacctcctcctgcctccaccaatcgg

SEQ ID NO: 13 - DNA, 21822 bp, synthetic construct
  source 1..21822: mol_type = other DNA
gattaaagaaggcatccagcaagaactgcacaagaaacggagtcagccggagaacaaggagtggtcttccactgcctcacaggaggatggaggcccacaacgcgtctgccccattcaacttcaccctgccacccaactttggcaagcgccccacagacctggcactgagcgtcatcctggtgttcatgttgttcttcatcatgctctcgctgggctgcaccatggagttcagcaagatcaaggctcacttatggaagcctaaagggctggccatcgccctggtggcacagtatggcatcatgcccctcacggcctttgtgctgggcaaggtcttccggctgaagaacattgaggcactggccatcttggtctgtggctgctcacctggagggaacctgtccaatgtcttcagtctggccatgaagggggacatgaacctcaggtaggcctgggggtgaggagggaacagcctgggacaggagctacattagatatgtcacaattgaggggaagagctgggctaaagccaggaaaaggagtgaatgcaggctgggtggaggctagggcaggggaagtctggcatttattgccaagagcttatgctgggatgggggcaggagttggggtacgtctggtttaggcaccaagccaaagggaccatactggtccaggaagctcgtggtgatctccttgcttccagaggtgtctcaaggattgtcttaaaggagcactgattaaagccaaaggaacctctggagaactgcatccacctacaacagtggacaagttgcctgctttgagcccagagatttaaaaggtagtgtgattcgttccaaacatcatcagggagatcaaggtgacttggaaccaaatgagcacctcaaattcctgcaaagcgggcaagaatttccaaaaggtcctcccaattacctggtcatctaagatgttttctctttaaagtccctcattccccatcagatgcaatgacatttccatgcaaaggcacaaccagggagatcctagagtttcccagcacccactccagataggccagccccatctcagccaccctgataaggggaagacagcagcaccccctaaccccctgcccaaagcattataagcagaaatcagcaagggctcgctcctggagacgcagcacacaggggctggttgatgctcatccttccaaactggggagactctagtcttgaacatctgccagaggagctacttcagacctgctccagctccagcagcctgtgctgaagctaaagaaaactcactagcagaagctcttgctttcagtgaaacctcagtctcaagccagtcccccctggtttagtcctccgtggagacgctattcaatgccaggtatagcagcagaacccagaaagtccatctgcttgtgtctgtcagcaccctactcctagccaaactttcctaggaaaactaacccagtcaagaagcccctcctagtgatgcaaaatgacactgggacagattttttaaatgaaaaaagtgagaatggctgggcacggtggctcacacctataatcccagcactttgggaggccaaggcaggaagatcacttgagaccaggagtttaagaccaacctgggcaacatagcaaaaccctgtgtctacaaatatatacatatatatttaattagccaggcatagtggcacatgcctgtagtcccagctacttgggaggctgagatgagaggatctttgagcctacgagttcaaggctacagtgagtcatgatcacaccactgcactccagcctgggcaacagagtgagaccctgtctctctctcaaaaaaaaaaaaaaaaaggaaataaaaaagtgagagtgaatgagagttggggtttaacaggatctgccatctgagaagctctgggctctttttccatatgattcagccctcactttagtagtcaatgggaagagaggcacaaagagggcatttggcttgtgccatcacccacggaaaggtatttcaatgttcaactacctcactgtgagaaaatgctttcctctgtctaatccacatcctctctgctacaaagcaagcccttggtcttggatctggcttctggacaaggctcatggcagcctgtgtgtcccaagaatcctagagtatatctgaattttggtgtatgagggatgcagaagtcaggggcctgcgaaggggtactctgagggctttgtgtttctgtggaagaagcagagcaggggacgtggaaggcaactgtgcttttaagtgtgagtggccagatgtcccagccccatcgccagtggccactattgaggcctctgccacaatgcctacattgctgtggctctaggccccacccaggcagagcctctggtgactcaagaacatgacccctcatgctctcctgctcaacctccttgtccagacccctgatgactgtcatttttacctagacccccacaagagataagctctgctgggacagccaccttctgccataaaactccttcccaacaaacaaccctcccatgcacattaaaccatgtctcagatcccactgtcggaatgggaatctggtcatctaccttcatgtgctgccagacttcaaatcccattccaatttgggacaacttgttacatgcattataataatgacaacgattataagaaacagccaggcacagtgctaaacaccttacctggataagtatatgtgatcctcctacagcaccctgggggacagcatgtaacaagtagcatttagaggtaaggaaactgaatcttagagaggtcaatacattggctccaggtctgggtggctccggaacctgtgcttttactgaaatatcttccaggtcagcatttccacaaacaaaatggccagacagcctcaaaggcatgggcagacacccgtgtctcagcagcaagtattgtgtgggagccagagcttcccacttttcaaaagaaggtaaaatatccattttatgtgaaatcagcaactcatccatttttttaaatgtgcaaaccaaacaatacatgtcaaagggccagctgtagcccaaaggccccagtttgagataagcacaatactctacatcccatttagaactggaaggaattgtaagacacagatcccattttacacatgaagaggctgaggtcagagacttgccagaggtttcccagagtttgtgggaaactgtgtctccctccactcactctgcctcataggtggagtcagtctggggacagcctgccatcccaggaggccaggccaccaagcagacccctcaatgcagccagggacggcagctccctcctcaaggaaggccactagacaagtttcaaaaaatgtaggtgacaaacaggcaagagaaaacgccctcacagttgaagtgagtatggcttgtcaggcacagagaagagagtgctatgccagcaggaactggcctcttttgaccatccttgctgccccaagttcatctgctccccctgccaccatcggactcacagcagagcccagagccaatggccatggggacaatgggcttggacacaacccaagcacttccacaggtctcccagccatgatgtaggaagctcaaccaggacaagatggaagccactggggtcccagaagttttccacttcatagagactgtgttgaggcccctcacagctccctcagcttctgcctccaatctgtgcattgacggacactcgaacgagtcccttaaatggggtacacagatgtccaaaagcaaggtctatgctgtgctcttggaacttaagcaggagggaaaaataaaaaaaacaaatgtgcccctttccccctgaaaggcaaccaggcctacaatctcaggaatctagagtctgaaaaagtccttgcccgtccctgtcccttggggtaaaccaaggatcacaacagtcacggatgccagcaggctaggagatggatgtccctgacctgaccccaggacctgtgcaagtggaggaagtacctggggatcagggaacatggctcctgcatacccaagctgacaaggcacaccaagtccaaactccaaaactcctgacacacaagccaaagtaagcatgattcctctgctctaggtcaccagaacaaacacacaagttgtgtttggcttttccctaagagaagatcaacagtagcattgaaaccccctgtattcctattcccaatctcattaccctccaccacttgccaaagggagcatccctgggaatttggtgttcagcatttccaccgacccttttcaactattatatacgtatatattcataaacaagatagactgcgggatttacgtcttttaattttgtacgaacagtattatactgtccacatcacctaacagcttgctttttttttttttttttgagaaataggcatgtaaagaaaatagctacctcatgtatctgaactgctgtgcagtttgcttgcatttatccattctcctgttgtcagattcatgtggattttacgtctcgcttctgtgaacagacttgtgtgtgtccctctgcccacatgagggcatatgcaattgaagcaggactgctgggtcgtgggagtcacacatctttaagaggtctccaaggtcattgttcacttcacacgttccctagcagtgtctgaagatccctgtttctctgtgccttcatctatatttggaattgtcagactttttaattttttccaaatgaaaacagtgaagcagatcgcagtagttttaaatttgatttcatgggaggcctaccacatttccacgtgtgcgttggccattcatgtgtcctcttctggaaacattgggtcctttgcctgtttttctatgggattgtggtttgggtgggttttgaaaattaatttctaaagttattgttttatatatgttgcaacagtctattcacagtttggagctggtctttacactttgataataatgtctgatagattgcttatgttaatttagtcaaatttgccaatatttttcctttgtggtttatgttttttaggggtttctttaagaaatcttttactatctacaagcctgtccaacatggcaaaaccccgtctctactaaaaaatacaaaaaaattagctgggcatggtgatgtgcacctgtaatcccagctactcaggaggctgaggcatgagaatcacttgaacccaggaggcagaggttgcagtgagccaaaatcatgccactgcactccagcctgggcgacagagcaacaccctgtctcaaaaaaaaaaaaaaaatttactgtctacaaagttacaaagatattaccctgtattttctttgaaaagttttaaatgtttgattttcacatggagagctccaatcaatgtagaacttatttttatgtatggcatgaactaattttatcttttccatacaaatagaagattccaagccacgttgagtgaatttttctccaggtatttgtaatgacatctctctcataaatgcagggcttgtgtctggatctctcttctcttctacgggtctatgtggctacccttgtaccagttccacactttcttaattacaattttataagttttgttatctgaaagttaagtccccctactttgttcttgaaaatcatgggccgggcacagtggctcacgcctgtaatcccagcactttgggaggtcaaggctggccgatcacgaggtcaggagatcgaaaccatcctggctaacacggtgaaaccccgtctctcctaaaaatacaaaaaattagctgggcgtggtggcgggcgcctgtagtcccagctacttgagaggctgaggcaggagaatggcgtgaacccgggaggcagagcttgcagtgagccaagatcacaccactgcactccaacctgggtgacagagcgagactctgtctcaaaaaaaaaaaaaatcatgttagctattcttagccttttactctttcacttgagtttcagtatcacctttcaagtctacaaaaaaaattataagtctggaagttttattaaatgtgcattacatttatatataaattgagagactatctttataatattgaatcttcccatctataaatatgatatagcttttcttttacttaggccttttttaatacctttctataaaggtttgtaatttctttcataaagatcttacatgtcctttagatgtataagtattttctagttttgtgaatatcataaatggtgtcttttaaaatatatatatatttgtttctagtatatcagacccaattgattgactgactgatgctaatgtatttagaacctcatacattttcttacttaccagttataatggaaaattacttagatttcttctgtggacaatcctatcattggcaaaacaagaaagtttcatttcttcttttcccatccctatactttttctatttcttgttttgctgtgcttactagggcctatatatagtataatttataaaacaggtaataataatgactatctttgctttattcctcttgttaaagaaaatccctctatttaccattaagtttaacgtttaatatactttttctaatagatttcctttcaagttaaggaattttcattccgttgctagtgtactaaatttttattatgtaagaacattaaattttgttgacttttctatagctacaaaattgtaccatttctttaataatatagtaatatgtattaatagattatctaatgttaaatcattcttactcatcttgttcattgtattttttaaaacacttatttgctaatattttattatttttgtttctatgttcataaataagagtggtccccaattttcttttctcgtaatatcattctccagctttcgttccaagatcatattaactttatacaataaattagggcatgtttcccctttttatattatctggacagatttgcataatattgcaacaatctgcactttgcatgtttagtagaacactaccgtaagacagtataaatcatgtagggttctagaagataggttttgaactgcttattaaatgtcttaaatgattctaagttgaatcaggttttcggcttcatattgacttggttttgtaggttatatttcccatgaaattgtccatttaagttaaaaatgaaagtatgaaaatgtccttaatgactttttaaaatcttcattttctctgtagttatgttccctttttcacactaacctcatcaatttgtgccatctctcttttccctggtctgccttgccagaggtgcctatttaattggcctttaaaaaaaaaaagaaaaaaaaaaaaccctatttttggttttactgattctaacaatttacaaaacttaaaaaatttcttccttttctttttattgggtttttctcattctaacctcttaaggtgcttaactcattaatttttaatcttctttttcaatataaatatgttaggctataaattgccctttaataattgctttatttgctccctgcatggtttttaattactatttattgagatataattcacataccataaaatccattctttaaattgtacgatgccttggtttttagcataattccaagcctgtgaacctatcaccattatctaatttcagaacattttcaataccccaaaaaggaactccatcccatttagcaggagtccccatttctctctcctgctccaacccttgacaaccactaatctttctgtctctatggatttcacataaatggaagcattcaatatgtgaccttttgtatctatacttctttcacttagcatattttcaaggttcattcatgtgatagcatgtcacaatacttcatttgtttttgtaattgaataatatcccattgtatggatataatacattttgctcatccattcattcagctgatggacacatgggttgtttctacttgttccctagtatgaataatgtctatggaagctcacaggcaagtttttgtgtgaacatgctttctgttcccttgggatatatacctaagagtagaattgctgggttatatggtgactctgtgtttaaattttctagaaactgccaaactattttccaaagcaccattttacttcccaccaccagtgtatgaggattcctaattttccatataccctccaacacttgttattgtccagctttttaattatagccatgctagtagatataaagtggtatctcaaagtggttttgatttgcattttcctaataagtaatgatgttgagcatcttttcatgtgctcattagccatttgtatatcttctttgggaaaatatctcattcaaatccttggcccattttttacttgttttttattattgcattataagagttcttcatatattggccgggcgcggtggctcacgcctgtaatcccagcactttgggaggcccaggcgggtggatcacgaggtcaggagatcgagactatcctggctaacacggtgaaaccccatctctactaaaaatacaaaaaaaattagccgggcgtggtggcaggcgcctgtagtcccagctactcgggaggctgagacagaagaatagcgtgaacccaggaggcggagcttgcagtgagccgagactgcgccactgcactccagcctgggcgacagagcaagactccatctcaaaaaaaaaaaaaaagagttcttcatatatattctgaatattagacccttatcagatatatgacttgcaaatatttcctcccattctcttgttggcttttcactttcttgatggtgtcctttgaatcacaagtttttaatattaatgaagtctgacttgcctttttctttggctgttcgtgctttagatgtctaagaaaccattgcctaatccaaagtcccctctccatgttttttaaatatgtattttattatttttatattctaaatactgtttccaccatgatttcatatttgatgatataattagaaagttcccctcccctcatcccatgatccttttattggttgctcagtttcaatgctcctcccatcccaggtctaatacctcaattctggtcccttccttgggcattaaagcctccacagtgccaagactaacaccccactcccctccagaacccctctatgaagcttcagcttgagttcccccactactttgcatttgtttcttgtccgtgggcacctttttttttagtggagtggcgctaacatagctcactgtaacctcaaccttctgggctcaagccatcctcctgcctcagcctcccaagtagctgggactcctggtgcacaccaccacacctggctaatttttgtgtgtggtttttggtttgtttgttcatttgtttgtgtgtagagacaggggtcttactttgttgcccaggctggtctcaaactcctggcttcaagcaatcctccctcctcggcctccctaaatgctggaattataggcatgagccactatgccacacttaaattgagtgttaatttcttacaagctcaagtgtgtatttaagtgttcacgggggtgggggagttcctattacctagttcacaattttctttgagggactcttctcacctctaacagctcttatccttatgacatgcctacctattgttcttgatggctacgctctggaacctttgatataggctgctcttcatgcatatttaagccatattccatctttatagaaataatgaaagaagtgtagatggagggtagggctgtggggaggaggagggacacactgagtgtggttttcctaagatcttgccaagccctcacctcccaaccatcttcccactggaataaaagtcagagccatatattgttaatgtaaatgtccataaatctaaggagatgccctatcagcttccttctcagttgacctttctggagggcctctccctagacagggtcctgggactaacatatataagattccaaatggaaaaggaaattggacaagtaggatacatattagagctggaaggacatattattcaggctctgagtgtcataatttattttaaataatatcaagaggaaccagaaagtaaagaacatgagaacaccagagttggggactggaatactatccaagatgcaaagcaaatctgaccaggttatcttgtcacttactcaacatgggtatctattactgccttctctactaccccaccattatggcccttcaattattctgtttcttactgcttcactatttcttttacagtccttttaatcatggtatctgtttattgatttttctagtagggtttttgacttctgcctgagcatcactcccatgccacccatccccaaagaccaagtgtcctgtcactgtgtctcacactcagactccccacgaggtaaaatgtgctgatatagctaattgttattgtctccgttggacaaagtcactacaagaagccattccctaggctctaaaccagacatttagtaactgtgggtcaggtgccattccatggtccaatgggctaatgaccagagtaatggaatcacacagttggctttctacaaaatgggcttggcatgggggaggtgctcaatgcgaaaagggttccattacagagaaggagatcagaatggctccttccacaatgaagcacacagggattgaagcagttgaaccaccctaagttcaaatgctgccttggccacttactaccttgtgcgactttgagcaggacacaactcttctagtccccagcttccttgtcagcgaaaagaagatagtatgcttactgcctaatgttatggggaaaaggaactgaagccacatatgtcaagcacttagcacagtggcacttagcaggcactcaacaaaatagtctgtatttatgggctctctctctctcccctcttcccatgtctcccaccagcattgtgatgaccacctgctccaccttctgtgcccttggcatgatgcctctcctcctgtacatctactccagggggatctatgatggggacctgaaggacaaggtgccctataaaggcatcgtgatatcactggtcctggttctcattccttgcaccatagggatcgtcctcaaatccaaacggccacaatacatgcgctatgtcatcaaggtaagaacctgggaggcttgacaaattagggcaaaagagtaaaaacaccatatacaactataagatataatcattatattaataataaacatacatatgctcctactagatgctaagttttatgctatgcactctaaggattcactgtcaccagagtttgatatttgcagaaagtgaagtaacacatgaggctggcgtggtggctcacgcttataatcccaacactttgggaagttgaggcgggtggatcacttgaggtcagaagtttgagaccagcctgaccaacatgacgaaaccccgtctccattaaaaatacaaaaaaaaaaaaaaaattagccgggcatggtagcgagtgcctgcaatcccagctactcaggaggctgaggcagcagaatcgcttgaaccagggagggcaggggttgcagtgagccgagatcgcgccactgcactcccgcctgggccacagagcaagactccatctctaaaacgaaaaaaaaaaaaaaaagaaaagaaagaaaatgaagtaacacacgagagaaacaagatccaattccagtgtacacgcaagtatttgcagaaggcaaggagatgggaacagtgagggctggagtggtcaaagagccttattaaaaaggaagggcttgaggcatttaggtaaacctggggagaagcctgaatagagttcagcaggcactaagggcaaatttggctgatgtggcaagaagctagaggagacaggtagtgtacccattcccagcatttcaagagagggatggggtgggaagtggagtgaattaaggccacaaaggttttgagaagcactgtggtcacagacagggcatcatgccactgctttgtgtgcagaactcctagagtgcagctatggtctctccaccaatattttcaaccttggagtttggtccctgccagcctcccccttggctcactattcatcccccagggccaggaggtcatcctggtcctcaggaaggacttgacctcctgacctcctgtaccatctctgcccctggatcaatgcctctggcctggtctgcaaggcgtgctgctgagaaccccaaagtttggcagcagctttgggcctggaacaaagaagtcaagagtccagttttcaggggggttttattttcacactaccattgtctcaagagtagtttttgcccttgtcctgtcccagtgccatggccacagctccccttcccgcctgctggaggacatggaggaatcatccctcctcctttctgtggccagtcaccccacctcagccctggattgcaatcattctctctacccttgtactcaagagacctccttttattgattatgcccctggcccagcagcctcccctctctctcatttctaaaatatctctgtattggttcttcacaatcagctttgatacgaactcacatatcttctatcttcaaaaaacaaaacaaaaagtgtctctcactgggattttttgtggaatccacctcccattccacctaatatctccctttgctctccttcctagccaggttttaggagtagtccccctcactcctctctctctctgtcaatcgcttcctctcaaacaccttccatttcccccatgccaccgccagagctctctcatgagtgaccaggccctcagcaattcgtttccattatttgaatccctctcttctctccttggtctcccatccacccagcatcttgatgaccacctgctccactttctttgccaaatccaatggacatttctcaatccttccttgaccttctagcagcattggacataattgaccactgcctcctcaaaagccttctctgtcagctttagtgactctaagctctgctgctttcctcctacctctcatgaccttcagactcccttatgagttcctctggtcaaaggcacttctctgcattcagaccaagctttctgcctttctcaccagacctctcccagcacaggatctggtccctctcaggtctgcaattaccatctgcaaaccgagacttccagaacagcatcatcagatgtcaactctatcctcactccagaatccatttattatctaccaggtctttctatctgaatatacctcagacatttaaaactcaatatatgttaaagtcatctcctccccatgtggttatctcagtcggtggcactgtgtgtgtgcccagttgccccaaccagaaactggaagtcattctcaatgttcccttctccctcaccactcacacccatctaatcaatgactgtcgattccaccttctgttgctggaatccattcattcctctctatccttacctctcactagacaactcaaatggccttctcatccaacctcctcacccagttctaacctgagggtgaagatgtgaagtacaaactctatcatgtcactccactgcttaaagcctctcattgactccttccccctaggatagtttaaactccttaatgtggcacttcttgatttggtttcaactgacctcaccagtctcagctcttttctctttgcacgtgcccttctcttcacctggaacactcttcttcccaattctcaccccactcaccatctttcactggatcactcccactcatcctttaagtctcaacacaaatagcgcctcccccaggaagccttccttaacctcctaagcctgagtaagatgcttgtcctattcactcccataacacccaacactccagctaacccctgtagcaattattacactgcattaagaacatctgttcatttttattccccactcatttatcaagaagcaagaccataaggctgtcttaattaacactgaatccacagcacctatcacagtgcccccatagctaatgcctgagaataaataaaggaatgaagggcaagtcctgtctgctgctcttccacacaccagcagaaaaacaacatacacagagaaacatgtttgtgtgcatcatttcatttttttaagatagtttgtggggtgcataagattgcttttttaaaaaatcctatacacccataaacgatcttaaagaaatgcatataaaatataaagaatattaaaatgtgaagtgaccactgctatcatttaatgctactctgccctcctccattttataggtgggaaaactgggaaacagaataccaagtcatagtgctaatgacaactgatgtgagaacatgtctcttttctacctcagagtagctttaaaagtgtttctctgaggttgcaaaaagcagaatctcagaccctttttttttttttttttcgagacggagtctcactctgtcgcccaggttggagtgcagtggcgcgagctcggctcactgcaagctccacctcccgagttcacaccattctcctgcctcagcctcctgagtagctgggactacaggcgcccgccaccatacccggataattttttgtattttttagtaaagacggggtttcaccgtgttagccaggatggtcccgatctcccgaccttgtgatctccccgccttggcctcccgaagtgctgggattacaggcgtgagccaccacaccttgccgagaatctcagatcttaaagctggagaagcctttaagatgaagttcagtctcctgccttattcccaatttgcagatagagtaaccgaggcccggaaaagttacttggcagagccaggattatttcaaccagtctgttcctgttgtgttgcacattcactactcaatgccacagacgaaatgaagtcaaaatgaagccagagaacattaaaagggctaagaaataccttggctggaaaaacagagactagtaataagggactacataaaaggtaatctaaaaaacccaggcatgccctggacccattttgcccctggctaaacctccagggttaaaccaccacaaagtcaacagctacactgatcttcctatattccaaaactggaatcactcctgtgaggatgcagagactaacatgtgggtcaagctgcaaaagctaaggtttccttgcttctatggctcttcgaggactatgctcatccttcgtcaccctgtcccataaataatatagtgagtgccaaactggaaaacgaacaaatgattgtcctagcctcttacgcacacataaccctccttggaccttgcaaatcaaaataacccaactttcaataactagctctggcagcaggtatctgctcacaccgggttcaaccagttgcattcgctggtggaaggtcacatttagatataccaagatacaatttcaggcatgctcattttaaaaggcaattcaagtaatttttaacttacccatgcaagggtttatccaaaggctatgtggctaaaagggtgatgaatctgagaatgacatggtccaggagccactttcaccataagccatccaccactctttaataaggatcccaccatttcattgactcaacactctactcatgcatttcccacacctgctgctactgctgaactgacccctaactctcctttgtcttacaacttctgccaacttgtggctattgctcccagacctctgttctgacacttgtccctgctattgcttccctctctgtaatcctgcaaacttcctaaatgagagaatctgattatttctaccactcaccagcctgtgcgggccacctttgtgggtagactgtggacatgtcatctcatgtgtaagccaatggattggctgcccatgaatgagcactcatcccagttgtgagcagggcaatgaagtcaggtggtacaaaatgtggtagcctatggagaagcaactacccggggccacttgtctcagcaggagacaggggttgtagacaggataaatttccagagatggcagcagtgagagtgacaggaacctctagaatgttaattcctcccctttgagtaataacaggagttacagtttagtgagcggttcttatatgccaggtcctatgccgtcaacctcactcaggttgagtgactccccagagcttacacacctggagactagcagaggcagctttcccaccaggatcatatcaaattatgtgctcataaccacaactgtgcagtccccaagagtcagagaacatgagtgagagaataagagtgaatgcctatacagaagaagagggcttctggattcgtggacaattcctgtaactcaatgtctcctacattatccactccactcttgatagagattaaaaattatttgactgttgggtaaaggacaaattcgaaagacagaaatctaaaaggggaaatcaactaaaaatgcaaaggtaaactgagaagttcattagctaataagtcttttaataaaacttgaagcacatggttattcaagagatgtgtgaaagtccttcctatgctgggataggaccactaattggtataggtgatgttttatatgatcatatcagttgcaacaagtattaaaaacagaaacacaaagctgtgcagctgaaatttgagcaatataaatgttaaaatgtcgttaggacattatgaatgtacttaatgccactgaactgtacactcaaaagtgattaaaatggcaaactttgctatacatattttacttttttaaaatgttactaggaaatataaaatgcaacacatgtgaatgaaccaaaaaatgaaccaacacatgtaactattacaaaataacatggtaagtttgagaaaagttaagtatttctttactcaggagataaaagggacagtcttaaatttacacatacctgtttatcattttggaggacgcaattctctaaaatgcacactggaaagtacaaagtgcatggagtgcacatgcactttgaaaggttgcatcctatgttgcacaatgaaaaaccctataatttcattcagagtagacctgcatagtttccaatgatgttgttatcacccaagatatttctgaaagttcttttgggaacttccttcatattcaatttgcaggtcactcaagaaagtaggtttgtccccttattattagacatcattttgggtttaaaaccgtatttccgggcttgatcatccaccttgtttaccagctttgtttcaaatgactcttggcaatttccagaattgatcctaaaatggataaaatccctatctgtaatcaaggtatttgagacaaatatgtatgacaagttctcatgagtttcaaagtatgtggaacaatgacagtattgtaggaatgtgtatataagttccaaaaatacttgacttttaaataatatccattttgcaggagtcctgaatagcctcacgtaacacttcactgaaagtctgctatccagagatgtgtcaaaagcagtaactgtgcttggctgagtttgtaataatcaaagtgttaatcagctgtccccaagcttcctagctactgatacgaaatctaaaaagatgggctgggtgcagtggctcacacctgtaatcccagcactttggaaggccaaggcaggaggaccgctaagagtccaggagtaagaccagcctgggcaacatagtaagaccctgtctctaatgttaataattttaaaaaaatttaaagaaaggaatccaaaaagaagataaaggtctttcatattcctttccacttccagtgttttattgcaatgtttcccacctccaatttctacctgtgcttccccacctctgttcctctctatccccaactcttctaattgccttttcttgtcttcattcccagggagggatgatcatcattctcttgtgcagtgtggccgtcacagttctctctgccatcaatgtggggaagagcatcatgtttgccatgacaccactcttgattgccacctcctccctgatgccttttattggctttctgctgggttatgttctctctgctctcttctgcctcaatggacggtaggtattttttttttttttaagaaagggtctcactctgttgcccaggctggaggggagtagcacattctcagctcactgcagcctcaacctccagggtttaactgatcctcctgcctcagccccccaagtaactgggactataggcacaagccaccatgaccagctgatttaacttttctggagacagggttttaccatgttgcccaggctagttttgaactaatgagctcaagtgatccacctgcctcagccttccaaagtgctaggattacaggtgtgagccactgcatgcctggagataggtattattttaggtcccactgtcccatgggcttcattcttgtcctagtctttcaggaaacgaataagaagctaggaacctgaaatctgggcaagacactttcctggcaatatgttcagatgcactaactggacaagagactttagacttgaaaaaaattgaatgaaggtattgaacgggttaaaaatccagacaccaaaggtatccaaacaccagcagcaatagcaaagtacccctgtccagggtctagattccatctgctgcgaaactctttggtagcagcactgggacaaagttgctccctccagttccctctgagtgtatgtggggttttcttcaccggcacaggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaagggtgagtactgaaattatccccacttcaagttctgctccaatattagacctgagcctgccaacaagcaagtttctagcagggactttttggatagggctttaactcttatcaagtcactaagaaccaggtagccccaagaagcacccaagaagcattaaagatccaagactacttccatcaactgcactccatttcttccttctagctgggagaatgaaattgcagatgcagtttacctcataaaactgaagcaatttgggctgctttgtctcagtggggagacatcataggacaaaactgccttggacaactgaggttattagttgtgtgaaaggcctctattcgcaagagttggggaagtttccatttctcaggactaggttctcatttcctggaaccagatcagagccacagcaccactggcacctccgtatgaggaaaaacatatgaaagcaatgcaccctaatataacccagaataaagaaaaaaaaaaaagattcctcaactctagttacgcagtaaaggaaacttaagcatacggttgttaaacagtaactacctccacaataaacttaatatgatttatactttactctgatcactatggggttaacatagcatctcttctctaaaatgctcagagtattacctttcaaaataaaattgaacctcaagacagaccagagaacagagtgccttcctcaaagtgcactttaacaccaattttaaaaaaaaaaaaaaaaaaaaaaaaaaacaggacattcaaacccactttataacacatttcattataactctcaacattagtatattcttcctaagtacctacccaaatccttcaactgtcccttcatgtccatgaattgaagtccccttcaaaacctttctagaccctagggactgtgatttacaccctaggagaaatacacaactttccctattggaatttgttacattatactagcaagcaaatgccgattctctctccctttgcaattagctaagatgtaattaggttgccaagcagccaacacgaaacaagtacaggctgaccatacactagaaccattttagaagctttggttcaaagctacagtagagttgtgagaaagaatctactctgatgcttatctgagaggaacgcaagagatttgctgccctcttacaaaaagtgcacagaagtttctagaccaaccaaattcccagatttgaggttttagtaccagaaagacctgaagtgacctcaaaggacaatactctatggatctttagctctcaagacttgaaaagaactttcctcacagtgtatctcagaagaccccagtggaaacactaaatcaaagcaacgtatttctttggacctaatggccccaagaagctattctcaggggggaggaatctgtcaagtttttatacttttcattcctgcacctgatgggagaacaactctgtatagagaactctcccatctgtatagaaaggtgcaggaatcaaagctatcttcaaatttgcatcaccgcctacaccacggacaagaagaggtagatcaattgggggttggagggaagaactggaaactctcttggttagactttctcgaagatctttcctgcctgcctcctcattccctgaatgcaacacagaccttgcatacataaactcatctcctgttgttttactgtataacgaagttagaagtgaagtgatgatgaagacacaatagtctaattcattaggacttttactactagtactaccattagggagactgaggggaaagtgagtttaaagagtaaaccatgggagagacgacagagatggaaaatatatacttagcacactctagggctgagttggtatttttgtactttccagattaaacaagcttcattgttcactcctcttgttctctctctacacctgtaaccctttttaactttacagataaaacaaaaatgatctacacagctgccacaactgaagaaacaattccaggagctctgggaaatggcacctacaaaggggaggactgctccccttgcacagcctagcccttcccctggtggcctggattctggtcccaaagcaattctgaaagccagtgtggtaaactagagagagcagcaaaaacaccagtcttgcctgagtctttctccagcatttccagtacatctatcagaatcatcaagtcttggccgggaacacagacagggtgtctacccaagaagcctcacctatccccaacttagaatttgctacttattttaaagacttgttcagtgactgtaaactctatgaaaccagaaaccgaatctgcctcttgctgggatctctaaaagtgtctgataagcatcttaaagtcactcaattcaactaatcaatatatatgtttaacccattactcaaatacccaaatcccattccaagttttgtgacccaaaagagaaataaatgctcacaagtgctgtagaattaaacttcagaagttctaacctaaaaagttcagatcctattccttcccttttgacattattgggatgatgctcccgaaaagtcaaatttgacatcaagtatgcaaaagtgaacacagtaagatgcaatcaggcaaaacaaactcaaaaaatagctaatgaaatgaaaaaactgggcgaatgcatcatgttagtagaggaggaaaacttttgacaaggaaaaaccaggaaacaaacacatacattaacacaatgttacctcactaataatctcttttttaagttcagtaggtatttggttttaacacaaggcgcccccaaaactgggtacagcaaactactgccaaaatgagtcgtttcccaattcaaagaaaaatgttcaaatacctagaattcacaaatttcaaattgtctctaataaaatttaaacattttgcatatca

SEQ ID NO: 14 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 15 - protein, 191 aa, Homo sapiens
DSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 16 - DNA, 150 bp, synthetic construct
  source 1..150: mol_type = other DNA
aactctagaatgggacagaatctttctgtgcccaatcctctgggattctttccagaccaccagttggatccactattcagagcaaattccagcagtcccgattgggacttcaacacaaacaaggacaattggccaatggcaaacaaggta

SEQ ID NO: 17 - DNA, 149 bp, synthetic construct
  source 1..149: mol_type = other DNA
tggaattctaccttgtttgccattggccaattgtccttgtttgtgttgaagtcccaatcgggactgctggaatttgctctgaatagtggatccaactggtggtctggaaagaatcccagaggattgggcacagaaagattctgtcccat

SEQ ID NO: 18 - RNA, 23 nt, synthetic construct
  source 1..23: mol_type = other RNA
gattgagaccttcgtctgcgngg

SEQ ID NO: 19 - RNA, 23 nt, synthetic construct
  source 1..23: mol_type = other RNA
gattgagatcttctgcgacgngg

SEQ ID NO: 20 - RNA, 23 nt, synthetic construct
  source 1..23: mol_type = other RNA
gtcgcagaagatctcaatctngg

SEQ ID NO: 21 - RNA, 23 nt, synthetic construct
  source 1..23: mol_type = other RNA
gctcctaccttgttggcgtcngg

SEQ ID NO: 22 - RNA, 23 nt, synthetic construct
  source 1..23: mol_type = other RNA
gttatcgctggatgtgtctgngg

SEQ ID NO: 23 - RNA, 23 nt, synthetic construct
  source 1..23: mol_type = other RNA
gctatgcctcatcttcttgtngg

SEQ ID NO: 24 - RNA, 23 nt, synthetic construct
  source 1..23: mol_type = other RNA
gtcggaacggcagacggagangg

SEQ ID NO: 25 - RNA, 23 nt, synthetic construct
  source 1..23: mol_type = other RNA
gaagcgaagtgcacacggtcngg

SEQ ID NO: 26 - RNA, 23 nt, synthetic construct
  source 1..23: mol_type = other RNA
ggtctccatgcgacgtgcagngg

SEQ ID NO: 27 - RNA, 46 nt, synthetic construct
  source 1..46: mol_type = other RNA
tttaaatgtatacccaaagacaaaagaaaattggtaacagcggtaa

SEQ ID NO: 28 - RNA, 46 nt, synthetic construct
  source 1..46: mol_type = other RNA
tttatgcctacagcctcctagtacaaagacctttaacctaatctcc

SEQ ID NO: 29 - RNA, 46 nt, synthetic construct
  source 1..46: mol_type = other RNA
tttggtacagcaacaggagggatacatagaggttccttgagcagta

SEQ ID NO: 30 - RNA, 46 nt, synthetic construct
  source 1..46: mol_type = other RNA
tttcacctctgcctaatcatctcttgttcatgtcctactgttcaag

SEQ ID NO: 31 - RNA, 46 nt, synthetic construct
  source 1..46: mol_type = other RNA
tttggaagtaatgattaactagatgttctggataataaggtttaat

SEQ ID NO: 32 - RNA, 46 nt, synthetic construct
  source 1..46: mol_type = other RNA
tttggaagagaaacagttatagagtatttggtgtctttcggagtgt

SEQ ID NO: 33 - RNA, 46 nt, synthetic construct
  source 1..46: mol_type = other RNA
tttgtaggcccactcacagttaatgagaaaagaagattgcaattga

SEQ ID NO: 34 - RNA, 46 nt, synthetic construct
  source 1..46: mol_type = other RNA
tttagagagtaaccccatctctttgttttgttagggtttaaatgta

SEQ ID NO: 35 - RNA, 46 nt, synthetic construct
  source 1..46: mol_type = other RNA
tttctagggggaactaccgtgtgtcttggccaaaattcgcagtccc

SEQ ID NO: 36 - RNA, 46 nt, synthetic construct
  source 1..46: mol_type = other RNA
tttggccaagacacacggtagttccccctagaaaattgagagaagt

SEQ ID NO: 37 - RNA, 46 nt, synthetic construct
  source 1..46: mol_type = other RNA
tttatccaaaggttaccaaatatttaccattggataagggtattaa

SEQ ID NO: 38 - RNA, 46 nt, synthetic construct
  source 1..46: mol_type = other RNA
tttaatacccttatccaatggtaaatatttggtaacctttggataa

SEQ ID NO: 39 - RNA, 50 nt, synthetic construct
  source 1..50: mol_type = other RNA
cccctaccaactggtcggggtttctgggatcttgcagagtttggtggaag

SEQ ID NO: 40 - RNA, 50 nt, synthetic construct
  source 1..50: mol_type = other RNA
cccctaccaactggtcggggtttattcagcgccgacgggacgtaaacaaa

SEQ ID NO: 41 - RNA, 50 nt, synthetic construct
  source 1..50: mol_type = other RNA
cccctaccaactggtcggggtttatcttctgcgacgcggcgattgagacc

SEQ ID NO: 42 - RNA, 50 nt, synthetic construct
  source 1..50: mol_type = other RNA
cccctaccaactggtcggggtttacgccgcagacacatccagcgataacc

SEQ ID NO: 43 - RNA, 50 nt, synthetic construct
  source 1..50: mol_type = other RNA
cccctaccaactggtcggggtttacggtggtctccatgcgacgtgcagag

SEQ ID NO: 44 - RNA, 50 nt, synthetic construct
  source 1..50: mol_type = other RNA
cccctaccaactggtcggggtttctttgaagtatgcctcaaggtcggtcg

SEQ ID NO: 45 - RNA, 50 nt, synthetic construct
  source 1..50: mol_type = other RNA
cccctaccaactggtcggggttttgattaggcagaggtgaaaaagttgca

SEQ ID NO: 46 - RNA, 50 nt, synthetic construct
  source 1..50: mol_type = other RNA
cccctaccaactggtcggggtttgacctgcctcgtcgtctaacaacagta

SEQ ID NO: 47 - RNA, 50 nt, synthetic construct
  source 1..50: mol_type = other RNA
cccctaccaactggtcggggtttattaactgtgagtgggcctacaaactg

SEQ ID NO: 48 - RNA, 50 nt, synthetic construct
  source 1..50: mol_type = other RNA
cccctaccaactggtcggggtttcccgccttccatagagtgtgtaaatag

SEQ ID NO: 49 - DNA, 20 bp, synthetic construct
  source 1..20: mol_type = other DNA
tcatctgccggaccgtgtgc

SEQ ID NO: 50 - DNA, 22 bp, synthetic construct
  source 1..22: mol_type = other DNA
tcccgatacagagctgaggcgg

SEQ ID NO: 51 - DNA, 30 bp, synthetic construct
  source 1..30: mol_type = other DNA
ttcaagcctccaagctgtgccttgggtggc

SEQ ID NO: 52 - DNA, 20 bp, synthetic construct
  source 1..20: mol_type = other DNA
gagtgctgtatggtgaggtg

SEQ ID NO: 53 - DNA, 20 bp, synthetic construct
  source 1..20: mol_type = other DNA
tttggggcatggacattgac

SEQ ID NO: 54 - DNA, 19 bp, synthetic construct
  source 1..19: mol_type = other DNA
ctcctccagcttatagacc

SEQ ID NO: 55 - DNA, 17 bp, synthetic construct
  source 1..17: mol_type = other DNA
gtgagtgggcctacaaa

SEQ ID NO: 56 - DNA, 22 bp, synthetic construct
  source 1..22: mol_type = other DNA
acaactttggtatcgtggaagg

SEQ ID NO: 57 - DNA, 19 bp, synthetic construct
  source 1..19: mol_type = other DNA
gccatcacgccacagtttc

SEQ ID NO: 58 - DNA, 21 bp, synthetic construct
  source 1..21: mol_type = other DNA
ccgtctgtgccttctcatctg

SEQ ID NO: 59 - DNA, 29 bp, synthetic construct
  source 1..29: mol_type = other DNA
agtccaagagtyctcttatgyaagacctt

SEQ ID NO: 60 - DNA, 26 bp, synthetic construct
  source 1..26: mol_type = other DNA
ccgtgtgcacttcgcttcacctctgc

SEQ ID NO: 61 - DNA, 20 bp, synthetic construct
  source 1..20: mol_type = other DNA
tcatctgccggaccgtgtgc

SEQ ID NO: 62 - DNA, 22 bp, synthetic construct
  source 1..22: mol_type = other DNA
tcccgatacagagctgaggcgg

SEQ ID NO: 63 - DNA, 30 bp, synthetic construct; identical to SEQ ID NO: 51
  source 1..30: mol_type = other DNA

SEQ ID NO: 64 - DNA, 19 bp, synthetic construct
  source 1..19: mol_type = other DNA
gacaagaatcctcacaata

SEQ ID NO: 65 - DNA, 20 bp, synthetic construct
  source 1..20: mol_type = other DNA
catagaggttccttgagcag

SEQ ID NO: 66 - DNA, 23 bp, synthetic construct
  source 1..23: mol_type = other DNA
tacatcgtttccatggctgctag

SEQ ID NO: 67 - DNA, 22 bp, synthetic construct
  source 1..22: mol_type = other DNA
caactcctcccagtctttaaac